SEQUENCE LISTING (24 sequences)

SEQ ID NO: 1 - protein, 500 aa, Human
MAGGGGSSDGSGRAAGRRASRSSGRARRGRHEPGLGGPAERGAGEARLEEAVNRWVLKFYFHEALRAFRGSRYGDFRQIRDIMQALLVRPLGKEHTVSRLLRVMQCLSRIEEGENLDCSFDMEAELTPLESAINVLEMIKTEFTLTEAVVESSRKLVKEAAVIICIKNKEFEKASKILKKHMSKDPTTQKLRNDLLNIIREKNLAHPVIQNFSYETFQQKMLRFLESHLDDAEPYLLTMAKKALKSESAASSTGKEDKQPAPGPVEKPPREPARQLRNPPTTIGMMTLKAAFKTLSGAQDSEAAFAKLDQKDLVLPTQALPASPALKNKRPRKDENESSAPADGEGGSELQPKNKRMTISRLVLEEDSQSTEPSAGLNSSQEAASAPPSKPTVLNQPLPGEKNPKVPKGKWNSSNGVEEKETWVEEDELFQVQAAPDEDSTTNITKKQKWTVEESEWVKAGVQKYGEGNWAAISKNYPFVNRTAVMIKDRWRTMKRLGMN

SEQ ID NO: 2 - protein, 543 aa, Human
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 3 - protein, 514 aa, Human
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 4 - protein, 1233 aa, human
MANDSPAKSLVDIDLSSLRDPAGIFELVEVVGNGTYGQVYKGRHVKTVTAAIKVMDVTEDEEEEITLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGGPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEILQQQLLQEQAMLLHDHRRPHAQQQPPPPQQQDRSKPSFHAPEPKPHYDPADRAREVQWSHLASLKNNVSPVSRSHSFSDPSPKFAHHHLRSQDPCPPSRSEGLSQSSDSKSEVPEPTQKAWSRSDSDEVPPRVPVRTTSRSPVLSRRDSPLQGGGQQNSQAGQRNSTSSIEPRLLWERVEKLVPRPGSGSSSGSSNSGSQPGSHPGSQSGSGERFRVRSSSKSEGSPSPRQESAAKKPDDKKEVFRSLKPAGEVDLTALAKELRAVEDVRPPHKVTDYSSSSEESGTTDEEEEDVEQEGADDSTSGPEDTRAASSPNLSNGETESVKTMIVHDDVESEPAMTPSKEGTLIVRQTQSASSTLQKHKSSSSFTPFIDPRLLQISPSSGTTVTSVVGFSCDGLRPEAIRQDPTRKGSVVNVNPTNTRPQSDTPEIRKYKKRFNSEILCAALWGVNLLVGTESGLMLLDRSGQGKVYPLISRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELLHKPLLVDLTVEEGQRLKVIYGSCAGFHAVDVDSGSVYDIYLPTHIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCGRNDKVFFSSVRSGGSSQVYFMTLGRTSLLSW

SEQ ID NO: 5 - protein, 1239 aa, HUMAN
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEVLQQQLLQEQAMLLECRWREMEEHRQAERLQRQLQQEQAYLLSLQHDHRRPHPQHSQQPPPPQQERSKPSFHAPEPKAHYEPADRAREVEDRFRKTNHSSPEAQSKQTGRVLEPPVPSRSESFSNGNSESVHPALQRPAEPQVPVRTTSRSPVLSRRDSPLQGSGQQNSQAGQRNSTSIEPRLLWERVEKLVPRPGSGSSSGSSNSGSQPGSHPGSQSGSGERFRVRSSSKSEGSPSQRLENAVKKPEDKKEVFRPLKPADLTALAKELRAVEDVRPPHKVTDYSSSSEESGTTDEEDDDVEQEGADESTSGPEDTRAASSLNLSNGETESVKTMIVHDDVESEPAMTPSKEGTLIVRQTQSASSTLQKHKSSSSFTPFIDPRLLQISPSSGTTVTSVVGFSCDGMRPEAIRQDPTRKGSVVNVNPTNTRPQSDTPEIRKYKKRFNSEILCAALWGVNLLVGTESGLMLLDRSGQGKVYPLINRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELVHKPLLVDLTVEEGQRLKVIYGSCAGFHAVDVDSGSVYDIYLPTHIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCERNDKVFFASVRSGGSSQVYFMTLGRTSLLSW

SEQ ID NO: 6 - protein, 1212 aa, HUMAN
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEVLQQQLLQEQAMLLHDHRRPHPQHSQQPPPPQQERSKPSFHAPEPKAHYEPADRAREVEDRFRKTNHSSPEAQSKQTGRVLEPPVPSRSESFSNGNSESVHPALQRPAEPQVPVRTTSRSPVLSRRDSPLQGSGQQNSQAGQRNSTSSIEPRLLWERVEKLVPRPGSGSSSGSSNSGSQPGSHPGSQSGSGERFRVRSSSKSEGSPSQRLENAVKKPEDKKEVFRPLKPAGEVDLTALAKELRAVEDVRPPHKVTDYSSSSEESGTTDEEDDDVEQEGADESTSGPEDTRAASSLNLSNGETESVKTMIVHDDVESEPAMTPSKEGTLIVRQTQSASSTLQKHKSSSSFTPFIDPRLLQISPSSGTTVTSVVGFSCDGMRPEAIRQDPTRKGSVVNVNPTNTRPQSDTPEIRKYKKRFNSEILCAALWGVNLLVGTESGLMLLDRSGQGKVYPLINRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELVHKPLLVDLTVEEGQRLKVIYGSCAGFHAVDVDSGSVYDIYLPTHIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCERNDKVFFASVRSGGSSQVYFMTLGRTSLLSW

SEQ ID NO: 7 - protein, 1320 aa, HUMAN
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEVLQQQLLQEQAMLLECRWREMEEHRQAERLQRQLQQEQAYLLSLQHDHRRPHPQHSQQPPPPQQERSKPSFHAPEPKAHYEPADRAREVEDRFRKTNHSSPEAQSKQTGRVLEPPVPSRSESFSNGNSESVHPALQRPAEPQVQWSHLASLKNNVSPVSRSHSFSDPSPKFAHHHLRSQDPCPPSRSEVLSQSSDSKSEAPDPTQKAWSRSDSDEVPPRVPVRTTSRSPVLSRRDSPLQGSGQQNSQAGQRNSTSSIEPRLLWERVEKLVPRPGSGSSSGSSNSGSQPGSHPGSQSGSGERFRVRSSSKSEGSPSQRLENAVKKPEDKKEVFRPLKPAGEVDLTALAKELRAVEDVRPPHKVTDYSSSSEESGTTDEEDDDVEQEGADESTSGPEDTRAASSLNLSNGETESVKTMIVHDDVESEPAMTPSKEGTLIVRQTQSASSTLQKHKSSSSFTPFIDPRLLQISPSSGTTVTSVVGFSCDGMRPEAIRQDPTRKGSVVNVNPTNTRPQSDTPEIRKYKKRFNSEILCAALWGVNLLVGTESGLMLLDRSGQGKVYPLINRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELVHKPLLVDLTVEEGQRLKVIYGSCAGFHAVDVDSGSVYDIYLPTHIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCERNDKVFFASVRSGGSSQVYFMTLGRTSLLSW

SEQ ID NO: 8 - protein, 1166 aa, HUMAN
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEVLQQQLLQEQAMLLHDHRRPHPQHSQQPPPPQQERSKPSFHAPEPKAHYEPADRAREVPVRTTSRSPVLSRRDSPLQGSGQQNSQAGQRNSTSSIEPRLLWERVEKLVPRPGSGSSSGSSNSGSQPGSHPGSQSGSGERFRVRSSSKSEGSPSQRLENAVKKPEDKKEVFRPLKPAGEVDLTALAKELRAVEDVRPPHKVTDYSSSSEESGTTDEEDDDVEQEGADESTSGPEDTRAASSLNLSNGETESVKTMIVHDDVESEPAMTPSKEGTLIVRQTQSASSTLQKHKSSSSFTPFIDPRLLQISPSSGTTVTSVVGFSCDGMRPEAIRQDPTRKGSVVNVNPTNTRPQSDTPEIRKYKKRFNSEILCAALWGVNLLVGTESGLMLLDRSGQGKVYPLINRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELVHKPLLVDLTVEEGQRLKVIYGSCAGFHAVDVDSGSVYDIYLPTHVRKNPHSMIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCERNDKVFFASVRSGGSSQVYFMTLGRTSLLSW

SEQ ID NO: 9 - protein, 1239 aa, HUMAN [sequence omitted]

SEQ ID NO: 10 - protein, 504 aa, HUMAN
MAAQRRSLLQSEQQPSWTDDLPLCHLSGVGSASNRSYSADGKGTESHPPEDSWLKFRSENNCFLYGVFNGYDGNRVTNFVAQRLSAELLLGQLNAEHAEADVRRVLLQAFDVVERSFLESIDDALAEKASLQSQLPEGVPQHQLPPQYQKILERLKTLEREISGGAMAVVAVLLNNKLYVANVGTNRALLCKSTVDGLQVTQLNVDHTTENEDELFRLSQLGLDAGKIKQVGIICGQESTRRIGDYKVKYGYTDIDLLSAAKSKPIIAEPEIHGAQPLDGVTGFLVLMSEGLYKALEAAHGPGQANQEIAAMIDTEFAKQTSLDAVAQAVVDRVKRIHSDTFASGGERARFCPRHEDMTLLVRNFGYPLGEMSQPTPSPAPAAGGRVYPVSVPYSSAQSTSKTSVTLSLVMPSQGQMVNGAHSASTLDEATPTLTNQSPTLTLQSTNTHTQSSSSSSDGGLFRSRPAHSLPPGEDGRVEPYVDFAEFYRLWSVDHGEQSVVTAP

SEQ ID NO: 11 - protein, 427 aa, HUMAN
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 12 - DNA, 2909 bp, Human
tggccgcgggagccgggacggcgggccccgcttccggcccgggcgtcgtgcgtgacccagcggcgtcacagccgaggaagcggcccggccgggagggcggggagggcgcgcggcgatcggacacgatggcgggaggaggcgggagtagcgacggcagcgggcgggcagctggcaggcgggcgtcccgcagtagcgggcgggcccggcgggggcgccacgagccggggctggggggcccggcggagcgcggcgcgggggaggcacggctggaagaggcagtcaatcgctgggtgctcaagttctacttccacgaggcgctgcgggcctttcggggtagccggtacggggacttcagacagatccgggacatcatgcaggctttgcttgtcaggcccttggggaaggagcacaccgtgtcccgattgctgcgggttatgcagtgtctgtcgcggattgaagaaggggaaaatttagactgttcctttgatatggaggctgagctcacaccactggaatcagctatcaatgtgctggagatgattaaaacggaatttacactgacagaagcagtggtcgaatccagtagaaaactggtcaaggaagctgctgtcattatttgtatcaaaaacaaagaatttgaaaaggcttcaaaaattttgaaaaaacatatgtccaaggaccccacaactcagaagctgagaaatgatctcctgaatattattcgagaaaagaacttggcccatcctgttatccagaacttttcatatgagaccttccagcagaagatgctgcgcttcctggagagccacctggatgacgccgagccctacctcctcacgatggccaaaaaggctttgaaatctgagtccgctgcctcaagtacagggaaggaagataaacagccagcaccagggcctgtggaaaagccacccagagaacccgcaaggcagctacggaatcctccaaccaccattggaatgatgactctgaaagcagctttcaagactctgtctggtgcacaggattctgaggcagcctttgcaaaactggaccagaaggatctggttcttcctactcaagctctcccagcatcaccagccctcaaaaacaagagacccagaaaagatgaaaacgaaagttcagccccggctgacggtgagggtggctcggaactgcagcccaagaacaagcgcatgacaataagcagattggtcttggaggaggacagccagagtactgagcccagcgcaggcctcaactcctcccaggaggccgcttcagcgccaccatccaagcccaccgttctcaaccaacccctccctggagagaagaatcccaaagtacccaaaggcaagtggaacagctctaatggggttgaagaaaaggagacttgggtggaagaggatgaactgtttcaagttcaggcagcaccagatgaagacagtacaaccaatataacaaaaaagcagaagtggactgtagaagaaagcgagtgggtcaaggctggagtgcagaaatatggggaaggaaactgggctgccatttctaaaaattacccatttgttaaccgaacagctgtgatgattaaggatcgctggcggaccatgaaaagacttggcatgaactgaaacaggctttcatttccacagaattcacaggagcatggttcctaataatagcccctgatagtctgctctttctttctttttcttttttttttttttttgagacagagtctcgctctgtcacccaggctggagtgcagtggcgtgatctcggctcactgcgacctccgtctcccgggctcacgccattctcctgcctcagcctcccgagtagctgggactacaggcgcccgccatcacgcccggctaatgttttgtatttttagtagagacggggtttcaccgtgttagccaggatggtctcgatctcctgacctcgtgatccacccaactcggcctcccaaagtgctgggattacaggcatgagccaccgcgcctggcatctgctgtttctttcagaagctgggctgggatgagaattttgggcaacctccttcgacgtgggggaggtcccatttccacttcatcactgttggagatcatggagctaagaagcagagccaagtccacccatgtccttggcagagatgacaggcacacagcttgtgcagtgccagaatatcattagcgtttcccttctttagtggtttgcttaaatttaaatccctggtaatctgtagaaccttctcctaggaaatggtgaagtctattaggagccacttgtgactccatgacctgttaaaaccagcaatgtgagtattatttggagtaaatttgttccacgtcaagttctggccttctgatgcaaatgcaaaggaacttagtctgttatgaacccaggttgatgacagaccagtccttgtggaataagattccctttaaaaactctttagccagtcgtgacatcaaccctagacctgtctgccttggcatttgctgtcaacatctgctgggctatgtaggcaggttaatcctccacttctcatgtggttgaaccagtgtgttttttggtaaaatggtgattgtagataagattagttccctgatcccctgccccctgtcccctgcctcttttcccaattcccttccttatgctggacttttaaagcttaaaaaaaatccgattgaatataaatgcctaatttcattctttgtgaaatggttgcttcctcctgattccctaattgtgctgtgttcgtgtcttgcactggaattcaacattcccttctccttttgtactgtgttgtgcttgctgtctctcccggacacccttaaagactgtctttttagcaaaaaatttcagtaaagtgttttctgtaatctttttttaaaaaaaaaaaaaaa

SEQ ID NO: 13 - DNA, 2547 bp, Human
cttacaaggtacagtcctctgctcaggggggccaggagggtcttataggcatcattcaccagggtcgaatgcttctctgagaagtccttttcagtctgagacctctggctgaagaaatctgggtggacaagacgctgcagttgctggtacctgtgctggagcttcgctgtatcaactctgaaggaacggttgcagtccataaggctgaagtagtctcgagtggggtcaggtgcctgcagcgctcggcactgtgggcagaagaacctgtcctcccgcccggggccccatgggccgccgcagttccaacagcggggataattgcttcccgcctgcgacgcagcatcgcagcttagcggtctccttctgggaacccctgtcggccaaaacccccacacccggagcaaagccccggctctcccccgccacatctggccggcggcctatctagccgtggtcactcgtggggaaaagcaaagagagcgtctaaccagactaatgttgctgattggctggggagtcgagggggcgggatcacccgaggggaacccgggttctaagttccgctctcccttctaaactacaactcccaggaggcattgaggcggcgcctgacggccacatctgctgctcctcattggtccggcggcaggggagggggttttgattggctgagggtggagtttgtatctgcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctgg

SEQ ID NO: 14 - DNA, 2460 bp, Human
cttacaaggtacagtcctctgctcaggggggccaggagggtcttataggcatcattcaccagggtcgaatgcttctctgagaagtccttttcagtctgagacctctggctgaagaaatctgggtggacaagacgctgcagttgctggtacctgtgctggagcttcgctgtatcaactctgaaggaacggttgcagtccataaggctgaagtagtctcgagtggggtcaggtgcctgcagcgctcggcactgtgggcagaagaacctgtcctcccgcccggggccccatgggccgccgcagttccaacagcggggataattgcttcccgcctgcgacgcagcatcgcagcttagcggtctccttctgggaacccctgtcggccaaaacccccacacccggagcaaagccccggctctcccccgccacatctggccggcggcctatctagccgtggtcactcgtggggaaaagcaaagagagcgtctaaccagactaatgttgctgattggctggggagtcgagggggcgggatcacccgaggggaacccgggttctaagttccgctctcccttctaaactacaactcccaggaggcattgaggcggcgcctgacggccacatctgctgctcctcattggtccggcggcaggggagggggttttgattggctgagggtggagtttgtatctgcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctgg

SEQ ID NO: 15 - DNA, 7171 bp, HUMAN
cacagagcgacagagacatttattgttatttgttttttggtggcaaaaagggaaaatggcgaacgactcccctgcaaaaagtctggtggacatcgacctctcctccctgcgggatcctgctgggatttttgagctggtggaagtggttggaaatggcacctatggacaagtctataagggtcgacatgttaaaacgggtcagttggcagccatcaaagttatggatgtcactgaggatgaagaggaagaaatcaaactggagataaatatgctaaagaaatactctcatcacagaaacattgcaacatattatggtgctttcatcaaaaagagccctccaggacatgatgaccaactctggcttgttatggagttctgtggggctgggtccattacagaccttgtgaagaacaccaaagggaacacactcaaagaagactggatcgcttacatctccagagaaatcctgaggggactggcacatcttcacattcatcatgtgattcaccgggatatcaagggccagaatgtgttgctgactgagaatgcagaggtgaaacttgttgactttggtgtgagtgctcagctggacaggactgtggggcggagaaatacgttcataggcactccctactggatggctcctgaggtcatcgcctgtgatgagaacccagatgccacctatgattacagaagtgatctttggtcttgtggcattacagccattgagatggcagaaggtgctccccctctctgtgacatgcatccaatgagagcactgtttctcattcccagaaaccctcctccccggctgaagtcaaaaaaatggtcgaagaagttttttagttttatagaagggtgcctggtgaagaattacatgcagcggccctctacagagcagcttttgaaacatccttttataagggatcagccaaatgaaaggcaagttagaatccagcttaaggatcatatagatcgtaccaggaagaagagaggcgagaaagatgaaactgagtatgagtacagtgggagtgaggaagaagaggaggaagtgcctgaacaggaaggagagccaagttccattgtgaacgtgcctggtgagtctactcttcgccgagatttcctgagactgcagcaggagaacaaggaacgttccgaggctcttcggagacaacagttactacaggagcaacagctccgggagcaggaagaatataaaaggcaactgctggcagagagacagaagcggattgagcagcagaaagaacagaggcgacggctagaagagcaacaaaggagagagcgggaagctagaaggcagcaggaacgtgaacagcgaaggagagaacaagaagaaaagaggcgtctagaggagttggagagaaggcgcaaagaagaagaggagaggagacgggcagaagaagaaaagaggagagttgaaagagaacaggagtatatcaggcgacagctagaagaggagcagcggcacttggaagtccttcagcagcagctgctccaggagcaggccatgttactgcatgaccataggaggccgcacccgcagcactcgcagcagccgccaccaccgcagcaggaaaggagcaagccaagcttccatgctcccgagcccaaagcccactacgagcctgctgaccgagcgcgagaggtggaagatagatttaggaaaactaaccacagctcccctgaagcccagtctaagcagacaggcagagtattggagccaccagtgccttcccgatcagagtctttttccaatggcaactccgagtctgtgcatcccgccctgcagagaccagcggagccacaggttcctgtgagaacaacatctcgctcccctgttctgtcccgtcgagattccccactgcagggcagtgggcagcagaatagccaggcaggacagagaaactccaccagcagtattgagcccaggcttctgtgggagagagtggagaagctggtgcccagacctggcagtggcagctcctcagggtccagcaactcaggatcccagcccgggtctcaccctgggtctcagagtggctccggggaacgcttcagagtgagatcatcatccaagtctgaaggctctccatctcagcgcctggaaaatgcagtgaaaaaacctgaagataaaaaggaagttttcagacccctcaagcctgctggcgaagtggatctgaccgcactggccaaagagcttcgagcagtggaagatgtacggccacctcacaaagtaacggactactcctcatccagtgaggagtcggggacgacggatgaggaggacgacgatgtggagcaggaaggggctgacgagtccacctcaggaccagaggacaccagagcagcgtcatctctgaatttgagcaatggtgaaacggaatctgtgaaaaccatgattgtccatgatgatgtagaaagtgagccggccatgaccccatccaaggagggcactctaatcgtccgccagactcagtccgctagtagcacactccagaaacacaaatcttcctcctcctttacaccttttatagaccccagattactacagatttctccatctagcggaacaacagtgacatctgtggtgggattttcctgtgatgggatgagaccagaagccataaggcaagatcctacccggaaaggctcagtggtcaatgtgaatcctaccaacactaggccacagagtgacaccccggagattcgtaaatacaagaagaggtttaactctgagattctgtgtgctgccttatggggagtgaatttgctagtgggtacagagagtggcctgatgctgctggacagaagtggccaagggaaggtctatcctcttatcaaccgaagacgatttcaacaaatggacgtacttgagggcttgaatgtcttggtgacaatatctggcaaaaaggataagttacgtgtctactatttgtcctggttaagaaataaaatacttcacaatgatccagaagttgagaagaagcagggatggacaaccgtaggggatttggaaggatgtgtacattataaagttgtaaaatatgaaagaatcaaatttctggtgattgctttgaagagttctgtggaagtctatgcgtgggcaccaaagccatatcacaaatttatggcctttaagtcatttggagaattggtacataagccattactggtggatctcactgttgaggaaggccagaggttgaaagtgatctatggatcctgtgctggattccatgctgttgatgtggattcaggatcagtctatgacatttatctaccaacacatatccagtgtagcatcaaaccccatgcaatcatcatcctccccaatacagatggaatggagcttctggtgtgctatgaagatgagggggtttatgtaaacacatatggaaggatcaccaaggatgtagttctacagtggggagagatgcctacatcagtagcatatattcgatccaatcagacaatgggctggggagagaaggccatagagatccgatctgtggaaactggtcacttggatggtgtgttcatgcacaaaagggctcaaagactaaaattcttgtgtgaacgcaatgacaaggtgttctttgcctctgttcggtctggtggcagcagtcaggtttatttcatgaccttaggcaggacttctcttctgagctggtagaagcagtgtgatccagggattactggcctccagagtcttcaagatcctgagaacttggaattccttgtaactggagctcggagctgcaccgagggcaaccaggacagctgtgtgtgcagacctcatgtgttgggttctctcccctccttcctgttcctcttatataccagtttatccccattctttttttttttcttactccaaaataaatcaaggctgcaatgcagctggtgctgttcagattctaccatcaggtgctataagtgtttgggattgagcatcatactggaaagcaaacacctttcctccagctccagaattccttgtctctgaatgactctgtcttgtgggtgtctgacagtggcgacgatgaacatgccgttggttttattggcagtgggcacaaggaggtgagaagtggtggtaaaaggagcggagtgctgaagcagagagcagatttaatatagtaacattaacagtgtatttaattgacatttcttttttgtaatgtgacgatatgtggacaaagaagaagatgcaggtttaagaagttaatatttataaaatgtgaaagacacagttactaggataacttttttgtgggtggggcttgggagatggggtggggtgggttaaggggtcccattttgtttctttggatttggggtgggggtcctggccaagaactcagtcatttttctgtgtaccaggttgcctaaatcatgtgcagatggttctaaaaaaaaaaaaaaaaaaaaaaaaaaaaggaaaaaaaaaaagaaaaagaaaacgtgtgcattttgtataatggccagaactttgtcgtgtgacagtattagcactgcctcagttaaaggtttaatttttgtttaaacctagacgtgcaacaaaagttttaccacagtctgcacttgcagaagaaagaaaaaaattcaaaccacatgtttattttttttttgcctacctcattgttcttaatgcattgagaggtgatttagtttatatgtttttggaagaaaccattaatgtttaatttaatcttaataccaaaacgaccagattgaagtttgacttttattgtcacaaatcagcaggcacaagaactgtccatgaagatgggaaatagccttaaggctgatgcagtttacttacaagtttagaaaccagaatgctttgtttttaccagattcaccattagaggttgatggggcaactgcagcccatgacacaagatctcattgttctcgatgtagaggggttggtagcagacaggtggttacattagaatagtcacacaaactgttcagtgttgcaggaaccttttcttgggggtgggggagtttcccttttctaaaaatgcaatgcactaaaactattttaagaatgtagttaattctgcttattcataaagtgggcatcttctgtgttttaggtgtaatatcgaagtcctggcttttctcgttttctcacttgctctcttgttctctgtttttttaaaccaattttactttatgaatatattcatgacatttgtaataaatgtcttgagaaagaatttgtttcatggcttcatggtcatcactcaagctcccgtaaggatattaccgtctcaggaaaggatcaggactccatgtcacagtcctgccatcttactttcctcttgtcgagttctgagtggaaataactgcattatggctgctttaacctcagtcatcaaaagaaacttgctgttttttaggcttgatctttttcctttgtggttaattttcctgtatattgtgaaaatgggggattttccctctgctcccacccacctaaacacagcagccatttgtacctgtttgcttcccatcccacttggcacccactctgacctcttgtcagtttcctgttcctggttccatctttttgaaaaaggccctcctttgagctacaaacatctggtaagacaagtacatccactcatgaatgcagacacagcagctggtggttttgtgtatacctgtaaagacaagctgagaagcttactttttggggaagtaaaagaagatggaaatggatgtttcatttgtatgagtttggagcagtgctgaaggccaaagccgcctactggtttgtagttaacctagagaaggttgaaaaattaatcctacctttaaagggatttgaggtaggctggattccatcgccacaggactttagttagaattaaattcctgcttgtaatttatatccatgtttaggcttttcataagatgaaacatgccacagtgaacacactcgtgtacatatcaagagaagaaggaaaggcacaggtggagaacagtaaaaggtgggcagatgtctttgaagaaatgctcaatgtctgatgctaagtgggagaaggcagagaacaaaggatgtggcataatggtcttaacattatccaaagacttgaagctccatgtctgtaagtcaaatgttacacaaaaaaaaatgcaaatggtgtttcattggaattaccaagtgcttagaacttgctggctttcccataggtggtaaaggggtctgagctcacaccgagttgtgcttggcttgcttgtgcagctccaggcacccggtgggcactctggtggtgtttgtggtgaactgaattgaatccattgttgggcttaagttactgaaattggaacaccctttgtccttctcggcgggggcttcctggtctgtgctttacttggcttttttccttcccgtcttagcctcacccccttgtcaaccagattgagttgctatagcttgatgcagggacccagtgaagtttctccgttaaagattgggagtcgtcgaaatgtttagattcttttaggaaaggaattattttccccccttttacagggtagtaacttctccacagaagtgccaatatggcaaaattacacaagaaaacagtattgcaatgacaccattacataaggaacattgaactgttagaggagtgctcttccaaacaaaacaaaaatgtctctaggtttagtcagagctttcacaagtaataacctttctgtattaaaatcagagtaaccctttctgtattgagtgcagtgttttttactcttttctcatgcacatgttacgttggagaaaatgtttacaaaaatggttttgttacactaatgcgcaccacatatttatggtttattttaagtgactttttatgggttatttaggttttcgtcttagttgtagcacacttaccctaattttgccaattattaatttgctaaatagtaatacaaatgacaaactgcattaaatttactaattataaaagctgcaaagcagactggtggcaagtacacagcccttttttttgcagtgctaacttgtctactgtgtattatgaaaattactgttgtccccccacccttttttccttaaataaagtaaaaatgacacctaaaaaaaaaaaaaaaaaaaaaaaaaaaaaaaaaaaaa

SEQ ID NO: 16 - DNA, 7495 bp, Human
cacagagcgacagagacatttattgttatttgttttttggtggcaaaaagggaaaatggcgaacgactcccctgcaaaaagtctggtggacatcgacctctcctccctgcgggatcctgctgggatttttgagctggtggaagtggttggaaatggcacctatggacaagtctataagggtcgacatgttaaaacgggtcagttggcagccatcaaagttatggatgtcactgaggatgaagaggaagaaatcaaactggagataaatatgctaaagaaatactctcatcacagaaacattgcaacatattatggtgctttcatcaaaaagagccctccaggacatgatgaccaactctggcttgttatggagttctgtggggctgggtccattacagaccttgtgaagaacaccaaagggaacacactcaaagaagactggatcgcttacatctccagagaaatcctgaggggactggcacatcttcacattcatcatgtgattcaccgggatatcaagggccagaatgtgttgctgactgagaatgcagaggtgaaacttgttgactttggtgtgagtgctcagctggacaggactgtggggcggagaaatacgttcataggcactccctactggatggctcctgaggtcatcgcctgtgatgagaacccagatgccacctatgattacagaagtgatctttggtcttgtggcattacagccattgagatggcagaaggtgctccccctctctgtgacatgcatccaatgagagcactgtttctcattcccagaaaccctcctccccggctgaagtcaaaaaaatggtcgaagaagttttttagttttatagaagggtgcctggtgaagaattacatgcagcggccctctacagagcagcttttgaaacatccttttataagggatcagccaaatgaaaggcaagttagaatccagcttaaggatcatatagatcgtaccaggaagaagagaggcgagaaagatgaaactgagtatgagtacagtgggagtgaggaagaagaggaggaagtgcctgaacaggaaggagagccaagttccattgtgaacgtgcctggtgagtctactcttcgccgagatttcctgagactgcagcaggagaacaaggaacgttccgaggctcttcggagacaacagttactacaggagcaacagctccgggagcaggaagaatataaaaggcaactgctggcagagagacagaagcggattgagcagcagaaagaacagaggcgacggctagaagagcaacaaaggagagagcgggaagctagaaggcagcaggaacgtgaacagcgaaggagagaacaagaagaaaagaggcgtctagaggagttggagagaaggcgcaaagaagaagaggagaggagacgggcagaagaagaaaagaggagagttgaaagagaacaggagtatatcaggcgacagctagaagaggagcagcggcacttggaagtccttcagcagcagctgctccaggagcaggccatgttactggagtgccgatggcgggagatggaggagcaccggcaggcagagaggctccagaggcagttgcaacaagaacaagcatatctcctgtctctacagcatgaccataggaggccgcacccgcagcactcgcagcagccgccaccaccgcagcaggaaaggagcaagccaagcttccatgctcccgagcccaaagcccactacgagcctgctgaccgagcgcgagaggtggaagatagatttaggaaaactaaccacagctcccctgaagcccagtctaagcagacaggcagagtattggagccaccagtgccttcccgatcagagtctttttccaatggcaactccgagtctgtgcatcccgccctgcagagaccagcggagccacaggtacagtggtcccacctggcatctctcaagaacaatgtttcccctgtctcgcgatcccattccttcagtgacccttctcccaaatttgcacaccaccatcttcgttctcaggacccatgtccaccttcccgcagtgaggtgctcagtcagagctctgactctaagtcagaggcgcctgaccctacccaaaaggcttggtctagatcagacagtgacgaggtgcctccaagggttcctgtgagaacaacatctcgctcccctgttctgtcccgtcgagattccccactgcagggcagtgggcagcagaatagccaggcaggacagagaaactccaccagcagtattgagcccaggcttctgtgggagagagtggagaagctggtgcccagacctggcagtggcagctcctcagggtccagcaactcaggatcccagcccgggtctcaccctgggtctcagagtggctccggggaacgcttcagagtgagatcatcatccaagtctgaaggctctccatctcagcgcctggaaaatgcagtgaaaaaacctgaagataaaaaggaagttttcagacccctcaagcctgctggcgaagtggatctgaccgcactggccaaagagcttcgagcagtggaagatgtacggccacctcacaaagtaacggactactcctcatccagtgaggagtcggggacgacggatgaggaggacgacgatgtggagcaggaaggggctgacgagtccacctcaggaccagaggacaccagagcagcgtcatctctgaatttgagcaatggtgaaacggaatctgtgaaaaccatgattgtccatgatgatgtagaaagtgagccggccatgaccccatccaaggagggcactctaatcgtccgccagactcagtccgctagtagcacactccagaaacacaaatcttcctcctcctttacaccttttatagaccccagattactacagatttctccatctagcggaacaacagtgacatctgtggtgggattttcctgtgatgggatgagaccagaagccataaggcaagatcctacccggaaaggctcagtggtcaatgtgaatcctaccaacactaggccacagagtgacaccccggagattcgtaaatacaagaagaggtttaactctgagattctgtgtgctgccttatggggagtgaatttgctagtgggtacagagagtggcctgatgctgctggacagaagtggccaagggaaggtctatcctcttatcaaccgaagacgatttcaacaaatggacgtacttgagggcttgaatgtcttggtgacaatatctggcaaaaaggataagttacgtgtctactatttgtcctggttaagaaataaaatacttcacaatgatccagaagttgagaagaagcagggatggacaaccgtaggggatttggaaggatgtgtacattataaagttgtaaaatatgaaagaatcaaatttctggtgattgctttgaagagttctgtggaagtctatgcgtgggcaccaaagccatatcacaaatttatggcctttaagtcatttggagaattggtacataagccattactggtggatctcactgttgaggaaggccagaggttgaaagtgatctatggatcctgtgctggattccatgctgttgatgtggattcaggatcagtctatgacatttatctaccaacacatatccagtgtagcatcaaaccccatgcaatcatcatcctccccaatacagatggaatggagcttctggtgtgctatgaagatgagggggtttatgtaaacacatatggaaggatcaccaaggatgtagttctacagtggggagagatgcctacatcagtagcatatattcgatccaatcagacaatgggctggggagagaaggccatagagatccgatctgtggaaactggtcacttggatggtgtgttcatgcacaaaagggctcaaagactaaaattcttgtgtgaacgcaatgacaaggtgttctttgcctctgttcggtctggtggcagcagtcaggtttatttcatgaccttaggcaggacttctcttctgagctggtagaagcagtgtgatccagggattactggcctccagagtcttcaagatcctgagaacttggaattccttgtaactggagctcggagctgcaccgagggcaaccaggacagctgtgtgtgcagacctcatgtgttgggttctctcccctccttcctgttcctcttatataccagtttatccccattctttttttttttcttactccaaaataaatcaaggctgcaatgcagctggtgctgttcagattctaccatcaggtgctataagtgtttgggattgagcatcatactggaaagcaaacacctttcctccagctccagaattccttgtctctgaatgactctgtcttgtgggtgtctgacagtggcgacgatgaacatgccgttggttttattggcagtgggcacaaggaggtgagaagtggtggtaaaaggagcggagtgctgaagcagagagcagatttaatatagtaacattaacagtgtatttaattgacatttcttttttgtaatgtgacgatatgtggacaaagaagaagatgcaggtttaagaagttaatatttataaaatgtgaaagacacagttactaggataacttttttgtgggtggggcttgggagatggggtggggtgggttaaggggtcccattttgtttctttggatttggggtgggggtcctggccaagaactcagtcatttttctgtgtaccaggttgcctaaatcatgtgcagatggttctaaaaaaaaaaaaaaaaaaaaaaaaaaaaggaaaaaaaaaaagaaaaagaaaacgtgtgcattttgtataatggccagaactttgtcgtgtgacagtattagcactgcctcagttaaaggtttaatttttgtttaaacctagacgtgcaacaaaagttttaccacagtctgcacttgcagaagaaagaaaaaaattcaaaccacatgtttattttttttttgcctacctcattgttcttaatgcattgagaggtgatttagtttatatgtttttggaagaaaccattaatgtttaatttaatcttaataccaaaacgaccagattgaagtttgacttttattgtcacaaatcagcaggcacaagaactgtccatgaagatgggaaatagccttaaggctgatgcagtttacttacaagtttagaaaccagaatgctttgtttttaccagattcaccattagaggttgatggggcaactgcagcccatgacacaagatctcattgttctcgatgtagaggggttggtagcagacaggtggttacattagaatagtcacacaaactgttcagtgttgcaggaaccttttcttgggggtgggggagtttcccttttctaaaaatgcaatgcactaaaactattttaagaatgtagttaattctgcttattcataaagtgggcatcttctgtgttttaggtgtaatatcgaagtcctggcttttctcgttttctcacttgctctcttgttctctgtttttttaaaccaattttactttatgaatatattcatgacatttgtaataaatgtcttgagaaagaatttgtttcatggcttcatggtcatcactcaagctcccgtaaggatattaccgtctcaggaaaggatcaggactccatgtcacagtcctgccatcttactttcctcttgtcgagttctgagtggaaataactgcattatggctgctttaacctcagtcatcaaaagaaacttgctgttttttaggcttgatctttttcctttgtggttaattttcctgtatattgtgaaaatgggggattttccctctgctcccacccacctaaacacagcagccatttgtacctgtttgcttcccatcccacttggcacccactctgacctcttgtcagtttcctgttcctggttccatctttttgaaaaaggccctcctttgagctacaaacatctggtaagacaagtacatccactcatgaatgcagacacagcagctggtggttttgtgtatacctgtaaagacaagctgagaagcttactttttggggaagtaaaagaagatggaaatggatgtttcatttgtatgagtttggagcagtgctgaaggccaaagccgcctactggtttgtagttaacctagagaaggttgaaaaattaatcctacctttaaagggatttgaggtaggctggattccatcgccacaggactttagttagaattaaattcctgcttgtaatttatatccatgtttaggcttttcataagatgaaacatgccacagtgaacacactcgtgtacatatcaagagaagaaggaaaggcacaggtggagaacagtaaaaggtgggcagatgtctttgaagaaatgctcaatgtctgatgctaagtgggagaaggcagagaacaaaggatgtggcataatggtcttaacattatccaaagacttgaagctccatgtctgtaagtcaaatgttacacaaaaaaaaatgcaaatggtgtttcattggaattaccaagtgcttagaacttgctggctttcccataggtggtaaaggggtctgagctcacaccgagttgtgcttggcttgcttgtgcagctccaggcacccggtgggcactctggtggtgtttgtggtgaactgaattgaatccattgttgggcttaagttactgaaattggaacaccctttgtccttctcggcgggggcttcctggtctgtgctttacttggcttttttccttcccgtcttagcctcacccccttgtcaaccagattgagttgctatagcttgatgcagggacccagtgaagtttctccgttaaagattgggagtcgtcgaaatgtttagattcttttaggaaaggaattattttccccccttttacagggtagtaacttctccacagaagtgccaatatggcaaaattacacaagaaaacagtattgcaatgacaccattacataaggaacattgaactgttagaggagtgctcttccaaacaaaacaaaaatgtctctaggtttagtcagagctttcacaagtaataacctttctgtattaaaatcagagtaaccctttctgtattgagtgcagtgttttttactcttttctcatgcacatgttacgttggagaaaatgtttacaaaaatggttttgttacactaatgcgcaccacatatttatggtttattttaagtgactttttatgggttatttaggttttcgtcttagttgtagcacacttaccctaattttgccaattattaatttgctaaatagtaatacaaatgacaaactgcattaaatttactaattataaaagctgcaaagcagactggtggcaagtacacagcccttttttttgcagtgctaacttgtctactgtgtattatgaaaattactgttgtccccccacccttttttccttaaataaagtaaaaatgacacctaaaaaaaaaaaaaaaaaaaaaaaaaaaaaaaaaaaaa

SEQ ID NO: 17 - DNA, 7033 bp, HUMAN
cacagagcgacagagacatttattgttatttgttttttggtggcaaaaagggaaaatggcgaacgactcccctgcaaaaagtctggtggacatcgacctctcctccctgcgggatcctgctgggatttttgagctggtggaagtggttggaaatggcacctatggacaagtctataagggtcgacatgttaaaacgggtcagttggcagccatcaaagttatggatgtcactgaggatgaagaggaagaaatcaaactggagataaatatgctaaagaaatactctcatcacagaaacattgcaacatattatggtgctttcatcaaaaagagccctccaggacatgatgaccaactctggcttgttatggagttctgtggggctgggtccattacagaccttgtgaagaacaccaaagggaacacactcaaagaagactggatcgcttacatctccagagaaatcctgaggggactggcacatcttcacattcatcatgtgattcaccgggatatcaagggccagaatgtgttgctgactgagaatgcagaggtgaaacttgttgactttggtgtgagtgctcagctggacaggactgtggggcggagaaatacgttcataggcactccctactggatggctcctgaggtcatcgcctgtgatgagaacccagatgccacctatgattacagaagtgatctttggtcttgtggcattacagccattgagatggcagaaggtgctccccctctctgtgacatgcatccaatgagagcactgtttctcattcccagaaaccctcctccccggctgaagtcaaaaaaatggtcgaagaagttttttagttttatagaagggtgcctggtgaagaattacatgcagcggccctctacagagcagcttttgaaacatccttttataagggatcagccaaatgaaaggcaagttagaatccagcttaaggatcatatagatcgtaccaggaagaagagaggcgagaaagatgaaactgagtatgagtacagtgggagtgaggaagaagaggaggaagtgcctgaacaggaaggagagccaagttccattgtgaacgtgcctggtgagtctactcttcgccgagatttcctgagactgcagcaggagaacaaggaacgttccgaggctcttcggagacaacagttactacaggagcaacagctccgggagcaggaagaatataaaaggcaactgctggcagagagacagaagcggattgagcagcagaaagaacagaggcgacggctagaagagcaacaaaggagagagcgggaagctagaaggcagcaggaacgtgaacagcgaaggagagaacaagaagaaaagaggcgtctagaggagttggagagaaggcgcaaagaagaagaggagaggagacgggcagaagaagaaaagaggagagttgaaagagaacaggagtatatcaggcgacagctagaagaggagcagcggcacttggaagtccttcagcagcagctgctccaggagcaggccatgttactgcatgaccataggaggccgcacccgcagcactcgcagcagccgccaccaccgcagcaggaaaggagcaagccaagcttccatgctcccgagcccaaagcccactacgagcctgctgaccgagcgcgagaggttcctgtgagaacaacatctcgctcccctgttctgtcccgtcgagattccccactgcagggcagtgggcagcagaatagccaggcaggacagagaaactccaccagcagtattgagcccaggcttctgtgggagagagtggagaagctggtgcccagacctggcagtggcagctcctcagggtccagcaactcaggatcccagcccgggtctcaccctgggtctcagagtggctccggggaacgcttcagagtgagatcatcatccaagtctgaaggctctccatctcagcgcctggaaaatgcagtgaaaaaacctgaagataaaaaggaagttttcagacccctcaagcctgctggcgaagtggatctgaccgcactggccaaagagcttcgagcagtggaagatgtacggccacctcacaaagtaacggactactcctcatccagtgaggagtcggggacgacggatgaggaggacgacgatgtggagcaggaaggggctgacgagtccacctcaggaccagaggacaccagagcagcgtcatctctgaatttgagcaatggtgaaacggaatctgtgaaaaccatgattgtccatgatgatgtagaaagtgagccggccatgaccccatccaaggagggcactctaatcgtccgccagactcagtccgctagtagcacactccagaaacacaaatcttcctcctcctttacaccttttatagaccccagattactacagatttctccatctagcggaacaacagtgacatctgtggtgggattttcctgtgatgggatgagaccagaagccataaggcaagatcctacccggaaaggctcagtggtcaatgtgaatcctaccaacactaggccacagagtgacaccccggagattcgtaaatacaagaagaggtttaactctgagattctgtgtgctgccttatggggagtgaatttgctagtgggtacagagagtggcctgatgctgctggacagaagtggccaagggaaggtctatcctcttatcaaccgaagacgatttcaacaaatggacgtacttgagggcttgaatgtcttggtgacaatatctggcaaaaaggataagttacgtgtctactatttgtcctggttaagaaataaaatacttcacaatgatccagaagttgagaagaagcagggatggacaaccgtaggggatttggaaggatgtgtacattataaagttgtaaaatatgaaagaatcaaatttctggtgattgctttgaagagttctgtggaagtctatgcgtgggcaccaaagccatatcacaaatttatggcctttaagtcatttggagaattggtacataagccattactggtggatctcactgttgaggaaggccagaggttgaaagtgatctatggatcctgtgctggattccatgctgttgatgtggattcaggatcagtctatgacatttatctaccaacacatgtaagaaagaacccacactctatgatccagtgtagcatcaaaccccatgcaatcatcatcctccccaatacagatggaatggagcttctggtgtgctatgaagatgagggggtttatgtaaacacatatggaaggatcaccaaggatgtagttctacagtggggagagatgcctacatcagtagcatatattcgatccaatcagacaatgggctggggagagaaggccatagagatccgatctgtggaaactggtcacttggatggtgtgttcatgcacaaaagggctcaaagactaaaattcttgtgtgaacgcaatgacaaggtgttctttgcctctgttcggtctggtggcagcagtcaggtttatttcatgaccttaggcaggacttctcttctgagctggtagaagcagtgtgatccagggattactggcctccagagtcttcaagatcctgagaacttggaattccttgtaactggagctcggagctgcaccgagggcaaccaggacagctgtgtgtgcagacctcatgtgttgggttctctcccctccttcctgttcctcttatataccagtttatccccattctttttttttttcttactccaaaataaatcaaggctgcaatgcagctggtgctgttcagattctaccatcaggtgctataagtgtttgggattgagcatcatactggaaagcaaacacctttcctccagctccagaattccttgtctctgaatgactctgtcttgtgggtgtctgacagtggcgacgatgaacatgccgttggttttattggcagtgggcacaaggaggtgagaagtggtggtaaaaggagcggagtgctgaagcagagagcagatttaatatagtaacattaacagtgtatttaattgacatttcttttttgtaatgtgacgatatgtggacaaagaagaagatgcaggtttaagaagttaatatttataaaatgtgaaagacacagttactaggataacttttttgtgggtggggcttgggagatggggtggggtgggttaaggggtcccattttgtttctttggatttggggtgggggtcctggccaagaactcagtcatttttctgtgtaccaggttgcctaaatcatgtgcagatggttctaaaaaaaaaaaaaaaaaaaaaaaaaaaaggaaaaaaaaaaagaaaaagaaaacgtgtgcattttgtataatggccagaactttgtcgtgtgacagtattagcactgcctcagttaaaggtttaatttttgtttaaacctagacgtgcaacaaaagttttaccacagtctgcacttgcagaagaaagaaaaaaattcaaaccacatgtttattttttttttgcctacctcattgttcttaatgcattgagaggtgatttagtttatatgtttttggaagaaaccattaatgtttaatttaatcttaataccaaaacgaccagattgaagtttgacttttattgtcacaaatcagcaggcacaagaactgtccatgaagatgggaaatagccttaaggctgatgcagtttacttacaagtttagaaaccagaatgctttgtttttaccagattcaccattagaggttgatggggcaactgcagcccatgacacaagatctcattgttctcgatgtagaggggttggtagcagacaggtggttacattagaatagtcacacaaactgttcagtgttgcaggaaccttttcttgggggtgggggagtttcccttttctaaaaatgcaatgcactaaaactattttaagaatgtagttaattctgcttattcataaagtgggcatcttctgtgttttaggtgtaatatcgaagtcctggcttttctcgttttctcacttgctctcttgttctctgtttttttaaaccaattttactttatgaatatattcatgacatttgtaataaatgtcttgagaaagaatttgtttcatggcttcatggtcatcactcaagctcccgtaaggatattaccgtctcaggaaaggatcaggactccatgtcacagtcctgccatcttactttcctcttgtcgagttctgagtggaaataactgcattatggctgctttaacctcagtcatcaaaagaaacttgctgttttttaggcttgatctttttcctttgtggttaattttcctgtatattgtgaaaatgggggattttccctctgctcccacccacctaaacacagcagccatttgtacctgtttgcttcccatcccacttggcacccactctgacctcttgtcagtttcctgttcctggttccatctttttgaaaaaggccctcctttgagctacaaacatctggtaagacaagtacatccactcatgaatgcagacacagcagctggtggttttgtgtatacctgtaaagacaagctgagaagcttactttttggggaagtaaaagaagatggaaatggatgtttcatttgtatgagtttggagcagtgctgaaggccaaagccgcctactggtttgtagttaacctagagaaggttgaaaaattaatcctacctttaaagggatttgaggtaggctggattccatcgccacaggactttagttagaattaaattcctgcttgtaatttatatccatgtttaggcttttcataagatgaaacatgccacagtgaacacactcgtgtacatatcaagagaagaaggaaaggcacaggtggagaacagtaaaaggtgggcagatgtctttgaagaaatgctcaatgtctgatgctaagtgggagaaggcagagaacaaaggatgtggcataatggtcttaacattatccaaagacttgaagctccatgtctgtaagtcaaatgttacacaaaaaaaaatgcaaatggtgtttcattggaattaccaagtgcttagaacttgctggctttcccataggtggtaaaggggtctgagctcacaccgagttgtgcttggcttgcttgtgcagctccaggcacccggtgggcactctggtggtgtttgtggtgaactgaattgaatccattgttgggcttaagttactgaaattggaacaccctttgtccttctcggcgggggcttcctggtctgtgctttacttggcttttttccttcccgtcttagcctcacccccttgtcaaccagattgagttgctatagcttgatgcagggacccagtgaagtttctccgttaaagattgggagtcgtcgaaatgtttagattcttttaggaaaggaattattttccccccttttacagggtagtaacttctccacagaagtgccaatatggcaaaattacacaagaaaacagtattgcaatgacaccattacataaggaacattgaactgttagaggagtgctcttccaaacaaaacaaaaatgtctctaggtttagtcagagctttcacaagtaataacctttctgtattaaaatcagagtaaccctttctgtattgagtgcagtgttttttactcttttctcatgcacatgttacgttggagaaaatgtttacaaaaatggttttgttacactaatgcgcaccacatatttatggtttattttaagtgactttttatgggttatttaggttttcgtcttagttgtagcacacttaccctaattttgccaattattaatttgctaaatagtaatacaaatgacaaactgcattaaatttactaattataaaagctgcaaagcagactggtggcaagtacacagcccttttttttgcagtgctaacttgtctactgtgtattatgaaaattactgttgtccccccacccttttttccttaaataaagtaaaaatgacacctaaaaaaaaaaaaaaaaaaaaaaaaaaaaaaaaaaaaa

SEQ ID NO: 18 - DNA, 3792 bp, HUMAN
atggcgaacgactcccctgcaaaaagtctggtggacatcgacctctcctccctgcgggatcctgctgggatttttgagctggtggaagtggttggaaatggcacctatggacaagtctataagggtcgacatgttaaaacgggtcagttggcagccatcaaagttatggatgtcactgaggatgaagaggaagaaatcaaactggagataaatatgctaaagaaatactctcatcacagaaacattgcaacatattatggtgctttcatcaaaaagagccctccaggacatgatgaccaactctggcttgttatggagttctgtggggctgggtccattacagaccttgtgaagaacaccaaagggaacacactcaaagaagactggatcgcttacatctccagagaaatcctgaggggactggcacatcttcacattcatcatgtgattcaccgggatatcaagggccagaatgtgttgctgactgagaatgcagaggtgaaacttgttgactttggtgtgagtgctcagctggacaggactgtggggcggagaaatacgttcataggcactccctactggatggctcctgaggtcatcgcctgtgatgagaacccagatgccacctatgattacagaagtgatctttggtcttgtggcattacagccattgagatggcagaaggtgctccccctctctgtgacatgcatccaatgagagcactgtttctcattcccagaaaccctcctccccggctgaagtcaaaaaaatggtcgaagaagttttttagttttatagaagggtgcctggtgaagaattacatgcagcggccctctacagagcagcttttgaaacatccttttataagggatcagccaaatgaaaggcaagttagaatccagcttaaggatcatatagatcgtaccaggaagaagagaggcgagaaagatgaaactgagtatgagtacagtgggagtgaggaagaagaggaggaagtgcctgaacaggaaggagagccaagttccattgtgaacgtgcctggtgagtctactcttcgccgagatttcctgagactgcagcaggagaacaaggaacgttccgaggctcttcggagacaacagttactacaggagcaacagctccgggagcaggaagaatataaaaggcaactgctggcagagagacagaagcggattgagcagcagaaagaacagaggcgacggctagaagagcaacaaaggagagagcgggaagctagaaggcagcaggaacgtgaacagcgaaggagagaacaagaagaaaagaggcgtctagaggagttggagagaaggcgcaaagaagaagaggagaggagacgggcagaagaagaaaagaggagagttgaaagagaacaggagtatatcaggcgacagctagaagaggagcagcggcacttggaagtccttcagcagcagctgctccaggagcaggccatgttactggagtgccgatggcgggagatggaggagcaccggcaggcagagaggctccagaggcagttgcaacaagaacaagcatatctcctgtctctacagcatgaccataggaggccgcacccgcagcactcgcagcagccgccaccaccgcagcaggaaaggagcaagccaagcttccatgctcccgagcccaaagcccactacgagcctgctgaccgagcgcgagaggtggaagatagatttaggaaaactaaccacagctcccctgaagcccagtctaagcagacaggcagagtattggagccaccagtgccttcccgatcagagtctttttccaatggcaactccgagtctgtgcatcccgccctgcagagaccagcggagccacaggttcctgtgagaacaacatctcgctcccctgttctgtcccgtcgagattccccactgcagggcagtgggcagcagaatagccaggcaggacagagaaactccaccagtattgagcccaggcttctgtgggagagagtggagaagctggtgcccagacctggcagtggcagctcctcagggtccagcaactcaggatcccagcccgggtctcaccctgggtctcagagtggctccggggaacgcttcagagtgagatcatcatccaagtctgaaggctctccatctcagcgcctggaaaatgcagtgaaaaaacctgaagataaaaaggaagttttcagacccctcaagcctgctgatctgaccgcactggccaaagagcttcgagcagtggaagatgtacggccacctcacaaagtaacggactactcctcatccagtgaggagtcggggacgacggatgaggaggacgacgatgtggagcaggaaggggctgacgagtccacctcaggaccagaggacaccagagcagcgtcatctctgaatttgagcaatggtgaaacggaatctgtgaaaaccatgattgtccatgatgatgtagaaagtgagccggccatgaccccatccaaggagggcactctaatcgtccgccggactcagtccgctagtagcacactccagaaacacaaatcttcctcctcctttacaccttttatagaccccagattactacagatttctccatctagcggaacaacagtgacatctgtggtgggattttcctgtgatgggatgagaccagaagccataaggcaagatcctacccggaaaggctcagtggtcaatgtgaatcctaccaacactaggccacagagtgacaccccggagattcgtaaatacaagaagaggtttaactctgagattctgtgtgctgccttatggggagtgaatttgctagtgggtacagagagtggcctgatgctgctggacagaagtggccaagggaaggtctatcctcttatcaaccgaagacgatttcaacaaatggacgtacttgagggcttgaatgtcttggtgacaatatctggcaaaaaggataagttacgtgtctactatttgtcctggttaagaaataaaatacttcacaatgatccagaagttgagaagaagcagggatggacaaccgtaggggatttggaaggatgtgtacattataaagttgtaaaatatgaaagaatcaaatttctggtgattgctttgaagagttctgtggaagtctatgcgtgggcaccaaagccatatcacaaatttatggcctttaagtcatttggagaattggtacataagccattactggtggatctcactgttgaggaaggccagaggttgaaagtgatctatggatcctgtgctggattccatgctgttgatgtggattcaggatcagtctatgacatttatctaccaacacatatccagtgtagcatcaaaccccatgcaatcatcatcctccccaatacagatggaatggagcttctggtgtgctatgaagatgagggggtttatgtaaacacatatggaaggatcaccaaggatgtagttctacagtggggagagatgcctacatcagtagcatatattcgatccaatcagacaatgggctggggagagaaggccatagagatccgatctgtggaaactggtcacttggatggtgtgttcatgcacaaaagggctcaaagactaaaattcttgtgtgaacgcaatgacaaggtgttctttgcctctgttcggtctggtggcagcagtcaggtttatttcatgaccttaggcaggacttctcttctgagctggtagaagcagtgtgatccagggattactggcctccagagtcttcaagatcctgagaacttggaattccttgtaact

SEQ ID NO: 19 - DNA, 3095 bp, HUMAN
gcccgcagggttcctccaagatggcggcgcagaggaggagcttgctgcagagtgagcagcagccaagctggacagatgacctgcctctctgccacctctctggggttggctcagcctccaaccgcagctactctgctgatggcaagggcactgagagccacccgccagaggacagctggctcaagttcaggagtgagaacaactgcttcctgtatggggtcttcaacggctatgatggcaaccgagtgaccaacttcgtggcccagcggctgtccgcagagctcctgctgggccagctgaatgccgagcacgccgaggccgatgtgcggcgtgtgctgctgcaggccttcgatgtggtggagaggagcttcctggagtccattgacgacgccttggctgagaaggcaagcctccagtcgcaattgccagagggagtccctcagcaccagctgcctcctcagtatcagaagatccttgagagactcaagacgttagagagggaaatttcgggaggggccatggccgttgtggcggtccttctcaacaacaagctctacgtcgccaatgtcggtacaaaccgtgcacttttatgcaaatcgacagtggatgggttgcaggtgacacagctgaacgtggaccacaccacagagaacgaggatgagctcttccgtctttcgcagctgggcttggatgctggaaagatcaagcaggtggggatcatctgtgggcaggagagcacccggcggatcggggattacaaggttaaatatggctacacggacattgaccttctcagcgctgccaagtccaaaccaatcatcgcagagccagaaatccatggggcacagccgctggatggggtgacgggcttcttggtgctgatgtcggaggggttgtacaaggccctagaggcagcccatgggcctgggcaggccaaccaggagattgctgcgatgattgacactgagtttgccaagcagacctccctggacgcagtggcccaggccgtcgtggaccgggtgaagcgcatccacagcgacaccttcgccagtggtggggagcgtgccaggttctgcccccggcacgaggacatgaccctgctagtgaggaactttggctacccgctgggcgaaatgagccagcccacaccgagcccagccccagctgcaggaggacgagtgtaccctgtgtctgtgccatactccagcgcccagagcaccagcaagaccagcgtgaccctctcccttgtcatgccctcccagggccagatggtcaacggggctcacagtgcttccaccctggacgaagccacccccaccctcaccaaccaaagcccgaccttaaccctgcagtccaccaacacgcacacgcagagcagcagctccagctctgacggaggcctcttccgctcccggcccgcccactcgctcccgcctggcgaggacggtcgtgttgagccctatgtggactttgctgagttttaccgcctctggagcgtggaccatggcgagcagagcgtggtgacagcaccgtagggcagccggaggaatgcagcccaagcagggcctggcatggggcaggacagggtccagccttttcctaacatctgcctgtgccacaacggccagcaggtgccccatcctctgcccacagcagactctgtcccatggctctccgggcagtagagtgtgtgagtgcagactggacctgtggttcataccttgtcaccacccgggaagctgaaggccacttcctcccagatggcctcagccaggaccatcgccctttctcagagcagagggccaggtagggaaaccgcagtgggcctgcaagccgcccgagcctccccagcagcctcctacagagcaggaagaggcgccctgtgaaccctgtagtgttgcaggcccagcagaccctgctgtcccaagcccacccctcctcccaccatcacctccctcacctcgggacagtagccctccacttctccagcctctcagccctgtgctcctgtatccagagtggaacccaggctggtgtccgtatctgtccctgggccccacccctggacctgccttggttgtgtcatctgttgtaaacattccaggaggaccaggggagcatctggggcctgggatggccacagaaggggcaggccaggtggaaaggagccagggggaagtggtctaagagacctggaactgccagaggatggcggcctgggcttccccagagccaggcgtgcgggagaggtgaggactggccccggtgggctgaggcaggggccgctgtcgtcaggcctgagccagggtgagctggtgcctgccttgcttcttccttctggtgctgtgaagaccataggctggcaggcagctgagatgaactgtctttaccactgatgaggggcctctgccggctgagggtagcaagcaggggttgtgagtcaggctgggggacttgtttgaaagaaagaggagttggaatgtggttcccaggagggaagaggttcctttgagacacagtaaccctgggaggcataggagaagggtcgggccagcccagcccagggcctgagttagactatttcccacatgttctctgccttcagtggggagggggtgccaccagggctgtcggccaggattgccactcctgtttcagaggaagcaggccgagagacttgcaccttggacaagccacacaatcagtggggcagccagagctcagacctgagccattgtgtcagtatccaggaccccccggattctccacgccctccccatctcccagtctccctgccccccatgccccagaccggcccaccagggactagccgctgtcgcacagcctctggggtgcttggtctctgcaaagtcaaaggcctgacagctctgtggcctgggaatccattttcctgcgggagagcagggcctggtgtggaaccagggagctgtgggaagccacagcagaaatggaagaaaaacaggtctcagcccagggtcctcgctcactccctcactccccactttgaagccatctctgttctgcaggtgagaggatttaaagtcagtcacaaaggcttgggaacaaaaggaatt

SEQ ID NO: 20 - DNA, 1412 bp, HUMAN
attaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgta

SEQ ID NO: 21 - DNA, 20 bp, HUMAN
cctgggctgccggctcgagc

SEQ ID NO: 22 - DNA, 20 bp, HUMAN
cgagctcggccgtcgggtcc

SEQ ID NO: 23 - DNA, 20 bp, HUMAN
cgtttacgtcgccgtccagc

SEQ ID NO: 24 - protein, 23 aa, ARTIFICIAL SEQUENCE
  Peptide
KKKVSRSGLYRSPSMPENLNRPR

(19) United States
(12) Patent Application Publication
(10) Pub. No.: US 2004/0242461 A1
(43) Pub. Date: Dec. 2, 2004

(54) Modulators of telomere stability

(76) Inventors: Michael D. Schneider, Houston, TX (US); Hidemasa Oh, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/820,583

(22) Filed: Apr. 8, 2004

Related U.S. Application Data
(60) Provisional application No. 60/461,095, filed on Apr. 8, 2003.

(51) Int. Cl. A61K 38/17

(52) U.S. Cl. 514/2

(57) ABSTRACT

The present invention embodies methods of modulating telomere repeat-binding factor-2 (TRF2) or cell cycle checkpoint kinase 2 (Chk2) to enhance the survival of a cell. More particularly, the modulators can be used to treat cardiovascular disease by improving the growth and survival of cardiomyocytes.

### CROSS-REFERENCE TO RELATED APPLICATIONS

This application claims priority to U.S. Provisional Application No. 60/461,095 filed on Apr. 8, 2003, which is incorporated herein by reference in its entirety.

### STATEMENT REGARDING FEDERALLY SPONSORED RESEARCH OR DEVELOPMENT

This invention was made with government support under NHLBI Grant Nos. RO1 HL47567 and RO1 HL60270 awarded by the National Institutes of Health. The United States Government may have certain rights in the invention.

### TECHNICAL FIELD

The present invention relates generally to the field of cell biology and medicine. In particular, the present invention relates to methods of modulating telomere repeat-binding factor-2 (TRF2) or cell cycle checkpoint kinase 2 (Chk2) to enhance the survival of a cell. More particularly, the modulators can be used to treat cardiovascular disease by improving the growth and survival of cardiomyocytes.

### BACKGROUND OF THE INVENTION

A. Telomeres

Telomeres, the protein-DNA structures physically located on the ends of the eukaryotic organisms, are required for chromosome stability and are involved in chromosomnal organization within the nucleus (Zakian, 1995; Blackburn and Gall, 1978; Oka et al., 1980; and Klobutcher et al., 1981). Telomeres are believed to be essential in such organisms as yeast and probably most other eukaryotes, as they allow cells to distinguish intact from broken chromosomes, protect chromosomes from degradation, and act as substrates for novel replication mechanisms. Telomeres are generally replicated in a complex, cell cycle and developmentally regulated, manner by telomerase, a telomere-specific DNA polymerase. In recent years, much attention has been focused on telomeres, as telomere loss has been associated with chromosomal changes such as those that occur in cancer and aging.

The single common structural feature of most eukaryotic telomeres is the presence of a tandem array of G-rich repeats which are necessary and sufficient for telomere function (Lundblad et al., 1989; Szostak et al., 1982). Although all telomeres of one genome are composed of the same repeats, the terminal sequences in different species vary. For instance, Oxytricha chromosomes terminate in TTTTGGGG repeats (Klobutcher et al., 1981), Tetrahymena utilizes an array of (TTGGGG)n (Blackburn et al., 1978), plant chromosomes carry the sequence (TTTAGGG)n (Richards et al., 1988), and trypanosomas and mammals have TTAGGG repeats at their chromosome ends (Blackburn et al., 1984; Brown, 1986; Cross et al., 1989; Moyzis et al., 1988; Van der Ploeg et al., 1984). The organization of the telomeric repeats is such that the G-rich strand extends to the 3′ end of the chromosome. At this position, telomerase, an RNA-dependent DNA polymerase, first demonstrated in Tetrahymena thermophila and other ciliates, can elongate telomeres, probably by using an internal RNA component as template for the addition of the appropriate G-rich sequence (Greider and 1985). This activity is thought to complement the inability of polymerases to replicate chromosome ends, but other mechanisms of telomere maintenance may operate as well (Pluta et al., 1989). Recently, it has been reported that the addition of telomerase into a cultured human cell leads to an increase of the proliferative life-span of that cell (Bodner et al., 1998).

Much less is known about the structure and behavior of chromosome ends of multicellular organisms. Mammalian telomeres have become amenable to molecular dissection with the demonstration that telomeric repeats of plants and T. thermophila species cross-hybridize to vertebrate chromosome ends (Allshire et al., 1988; Richards et al., 1988). It has also been shown that human DNA contains tandem arrays of TTAGGG repeats, probably at the chromosome ends, providing further evidence for the evolutionary conservation of telomeres and a tool for the isolation of telomeric DNA (Moyzis et al., 1988). Two strategies to obtain human chromosome ends have proven successful: an indirect isolation protocol that relies on human telomeres to be functional in S. cerevisiae (Brown et al., 1989; Cross et al., (1989) and direct cloning in E. coli.

TRF activity was first identified in 1992 by Zhong et al. (1992) as a DNA-binding factor specific for TTAGGG repeat arrays. TRF was found to be present in nuclear extracts of human, mouse and monkey cells. The optimal site for TRF binding was found to contain at least six contiguous TTAGGG repeats.

B. Cardiovascular Disease

Cardiovascular disease involves diseases or disorders associated with the cardiovascular system. Such disease and disorders include those of the pericardium, heart valves, myocardium, blood vessels, and veins. Myocardial infarction (MI) is a life-threatening event and may cause cardiac sudden death or heart failure. Despite considerable advances in the diagnosis and treatment of heart disease, cardiac dysfunction after MI is still the major cardiovascular disorder that is increasing in incidence, prevalence, and overall mortality (Eriksson et al., 1995). After acute myocardial infarction, the damaged cardiomyocytes are gradually replaced by fibroid nonfunctional tissue. Ventricular remodeling results in wall thinning and loss of regional contractile function. The ventricular dysfunction is primarily due to a massive loss of cardiomyocytes. It is widely accepted that adult cardiomyocytes have little regenerative capability.

Therefore, the loss of cardiac myocytes after MI is irreversible. Each year more than half million Americans die of heart failure. The relative shortage of donor hearts forces researchers and clinicians to establish new approaches for treatment of cardiac dysfunction in MI and heart failure patients.

The emerging concept of heart failure as a myocyte-deficiency disease is predicated on the limited regenerative capacity of mammalian cardiac muscle, which is inadequate to maintain pump function after cell death (MacLellan, W. R. et al., 2000; Zhang, D. et al., 2000; Oh, H. et al., 2001; Pasumarthi, K. B. et al., 2002.). Conceptually, approaches to augment cardiac myocyte number and survival include cell grafting (Koh, G. Y. et al., 1995), driving non-muscle cells to a cardiac “fate” (Grepin, C. et al., 1997), potentiating repair by endogenous stem cells (Jackson, K. A. et al., 2001), and alleviating apoptosis (Reed, J. C. et al., 1999). A rational approach to such interventions encompasses identifying endogenous molecules that contribute to cell survival in the heart (Hirota, H. et al., 1999; Kubasiak, L. A. et al., 2002; Sadoshima, J. et al., 2002; Yussman, M. G. et al., 2002).

Telomere maintenance is one mechanism through which cell viability is preserved (Lee, H. W. et al., 1998; Hahn, W. C. et al., 1999; Weinert, T. & Lundblad, V. et al., 1999; Wong, K. K. et al., 2000; Karlseder, J. et al., 1999; Hemann, M. T. et al., 2001; Stewart, S. A. et al., 2002; de Lange, T., 2002; Chang, S. et al., 2002). Telomeres consist of tandem T2AG3 repeats at chromosome ends, maintained by telomerase reverse transcriptase (TERT) and bound by specific telomeric repeat binding factors including TRF1 and TRF2 (Karlseder, J., 1999; de Lange, T., 2002; McEachem, M. J., 2000; Blackburn, E. H., 2001). It has been shown that TERT and telomerase activity are down-regulated in adult mouse myocardium (unlike some other adult tissues in the mouse (Prowse, K. R. & Greider, C. W., 1995)), and that forced expression of TERT in transgenic mice can delay the timing of cardiac myocytes' cell cycle exit (Oh, H., 2001). At later ages, continued expression of TERT at the level found in embryonic hearts had two other effects with possible therapeutic significance. First, TERT induced myocyte enlargement (hypertrophic growth), after the cessation of cycling. Second, TERT suppressed cardiac myocyte apoptosis both in vitro (serum starvation) and in vivo (ischemia-reperfusion injury).

Current therapeutic agents to combat heart failure include diuretics, ACE inhibitors, vasodilators, beta-blockers, digitalis, anticoagulants, left ventricular assist devices and transplantation. Numerous types of agents that fall in to these categories of therapeutic agents have been developed along with several derivatives of such therapeutic agents. One example of such derivatives is S-nitroso derivatives of ACE inhibitors (U.S. Pat. Nos. 5,187,183 and 5,118,180).

Even with so many treatment options, the survival rate of patients suffering from heart failure is less than 50% five years after diagnosis and less than 25% ten years after diagnosis. Therefore, there is a need to develop other techniques and therapeutic agents. The present invention is the first to develop new cellular targets for the treatment of cardiovascular disease. These two targets are TRF2 and Chk2.

### BRIEF SUMMARY OF THE INVENTION

The present invention embodies methods for controlling the cellular function of the telomere repeat-binding factor-2, TRF2, and the cell cycle checkpoint kinase 2, Chk2. More specifically the present invention relates to the cellular modulation of TRF2 and Chk2 in the context of cardiovascular disease and cardiomyocyte survival. Even more specifically, the present invention addresses the cellular modulation of apoptosis of cardiomyocytes by TRF2 and Chk2. No other invention describes the utilization of modulators of TRF2 and Chk2 in the control of cardiomyocyte apoptosis, cardiomyocyte survival, and cardiovascular disease.

One embodiment of the present invention comprises a method of enhancing the survival of a cell comprising the steps of administering to the cell a composition that regulates telomere stability in the cell. The cell is in a tissue, more specifically, the tissue is in a human. In specific embodiments, the cell is a cardiomyocyte. More specifically, the cell is under oxidative stress.

It is envisioned that the composition comprises a modulator of telomeric repeat binding factor-2 (TRF2). The modulator is telomerase reverse transcriptase (TERT). In further embodiments the modulator of TRF2 can be an inhibitor of hematopoietic progenitor kinase/germinal center kinase like kinase (HGK), HGK-related kinases and/or HGK-activated kinases, for example transforming growth factor β-activated kinase-1 (TAK1) and/or jun N-terminal kinase-1 (JNK1).

In further embodiments, the composition comprises a modulator of cell cycle checkpoint kinase 2 (Chk2).

Another embodiment of the present invention is a method of treating a subject suffering from a cardiovascular disease comprising the step of administering to the subject an effective amount of a composition to regulate telomere stability, wherein the effective amount increases cardiomyocyte survival.

The cardiovascular disease is selected from the group consisting of coronary artery disease, myocardial infarction, heart failure, ischemic heart disease, and angina. More specifically, the cardiovascular disease is myocardial infarction, which can be caused by arterial obstruction.

In certain embodiments, the cardiovascular disease is caused by oxidative stress on cardiomyocytes. More specifically, cardiovascular disease is caused by telomere loss in cardiomyocytes. The telomere loss results in apoptosis. The apoptosis is associated with check point kinase Chk2 activation.

In further embodiments, the modulator increases activity of TRF2, increases the expression of TRF2, increases the stability of TRF2, modulator inhibits Chk2 activity, reduces expression of Chk2, increases degradation of Chk2 and/or destabilizes Chk2. Yet further, the composition comprises an expression vector having a polynucleotide sequence encoding a TRF2 protein.

Another embodiment is a method of treating a subject suffering from a myocardial infarction comprising the step of administering to the subject an effective amount of a composition to regulate telomere stability, wherein the effective amount increases cardiomyocyte survival. In certain embodiments, the myocardial infarction is caused by arterial obstruction; oxidative stress on cardiomyocytes; or telomere loss and/or telomere dysfunction in cardiomyocytes. The telomere loss and/or telomere dysfunction can results in apoptosis, which can be associated with check point kinase Chk2 activation.

Yet further, another embodiment of the present invention is a method of treating heart failure comprising the step of administering to a subject an effective amount of a composition to modulate telomere stability. The method further comprises administering angiotensin II converting enzyme (ACE) inhibitors or diuretics.

Another embodiment comprises a method of treating a subject at risk for ventricular dysfunction associated with mechanical stress comprising the steps of administering to the subject an effective amount of a composition to modulate telomere stability, wherein the effective amount decreases ventricular dysfunction. The mechanical stress induces oxidative stress. It is envisioned that the composition attenuates telomere dysfunction. Yet further, the composition can comprises a modulator of TRF2 or Chk2.

A further embodiment comprises a method of regulating cardiomyocyte apoptosis in a subject having an myocardial infarction comprising the step of administering to the subject an effective amount of a composition to regulate telomere stability, wherein the effective amount increases cardiomyocyte survival.

Another embodiment is a method of regulating cardiomyocyte apoptosis in a subject at risk for heat failure comprising the step of administering to the subject an effective amount of a composition to regulate telomere stability, wherein the effective amount increases cardiomyocyte survival.

Still further, another embodiment is a method for regulating telomere stability in cardiomyocytes of a subject at risk for a cardiovascular disease comprising the step of administering to the subject an effective amount of a composition to regulate telomere stability. The composition enhances telomeric signaling.

Another embodiment is a method for regulating telomere signaling in cardiomyocytes of a subject at risk for a cardiovascular disease comprising the step of administering to the subject an effective amount of a composition to regulate telomere signaling. The composition enhances telomere stability.

Yet further, another embodiment is a method of regulating oxidative stress in a cardiomyocyte during mechanical stress comprising the steps of administering to the cardiomyocyte a composition to regulate telomere stability via a decrease in oxidative stress in the cardiomyocyte.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1A-FIG. 1D show telomere dysfunction in human heart failure. FIG. 1A, illustrates cardiomyocyte apoptosis, shown by TUNEL and sarcomeric MHC staining. FIG. 1B (left) shows cardiac telomere erosion with a Southern blot using a telomere-specific probe, (middle) telomere length as a function of age, and (right) that telomere erosion occurred without overt change in cardiac TERT or TERC mRNA levels. FIG. 1C shows loss of cardiac TRF2 protein in heart failure, by Western blot. FIG. 1D shows activation of Chk2 (Thr68 phosphorylation) in heart failure.

FIG. 2A-FIG. 2E depict dominant-negative TRF2 triggers telomere dysfunction and apoptosis in cardiomyocytes. FIG. 2A shows viral vectors TRF1 and TRF2 tagged with FLAG and myc epitopes respectively (upper left) and Western blots confirming expression of the exogenous proteins in cardiomyocytes (lower left). Irrunocytochemistry for the exogenous proteins in cardiomyocytes (right): TRF1/2, FITC; MF20, tetramethyl rhodamine isothiocyanate; nuclei, DAPI. Bar, 5 μm. FIG. 2B shows telomere shortening by Southern blot. FIG. 2C shows activation of Chk2, as illustrated by immune complex kinase assays. FIG. 2D demonstrates apoptosis shown as hypodiploid DNA by flow cytometry. FIG. 2E illustrates PARP cleavage, shown by Western blotting.

FIG. 3A-FIG. 3I show down-regulation of endogenous TRF2 in cardiomyocytes by antisense oligonucleotide or oxidative stress. FIG. 3A shows reduction of TRF2 specifically by antis-sense TRF2 by Western blot. Adenoviral delivery of GFP was used for all myocytes in the upper panel. FIG. 3B shows Chk2 activation by immune complex kinase assay. FIG. 3C demonstrates telomere shortening by Southern blot. FIG. 3D shows cardiomyocyte apoptosis by flow cytometry. FIG. 3E shows PARP cleavage by Western blot. FIG. 3F illustrates a Western blot showing rapid down-regulation of TRF2 by H₂O₂. Telomere shortening (FIG. 3G), PARP cleavage (FIG. 3H), and apoptosis (FIG. 3I) were each induced by H₂O₂ and rescued by viral delivery of TRF2 or TERT.

FIG. 4A-FIG. 4D show that TERT protects adult mouse myocardium from telomere shortening, apoptosis, fibrosis, and systolic dysfunction after biomechanical stress. Telomere length (FIG. 4A), TRF2 levels (FIG. 4B), and Chk2 kinase activation (FIG. 4C) were measured as in FIG. 2. FIG. 4D shows representative TUNEL and picrosirius staining, in banded mice. Mean results ±S. E. are shown for apoptosis (left), fibrosis (middle), and peak aortic ejection velocity by Doppler echocardiography (right).

FIG. 5A-FIG. 5G show HGK activates the mitochondrial death pathway. FIG. 5A shows uniform delivery of the viral vectors to cardiomyocytes. Expression was confirmed by indirect immunostaining with antibodies to the FLAG or HA epitope (FITC) and to sarcomeric α-actin (Texas Red). Bar, 20 μm. FIG. 5B shows ceramide activates HGK. HGK activity was measured by immune complex kinase assays, after treatment with 50 μg/ml C2-ceramide. FIG. 5C shows H₂O₂ activates HGK. Immune complex kinase assays were performed following treatment with 200 μM H₂O₂. FIG. 5D-FIG. 5G show lethality of HGK depends largely on its catalytic activity. FIG. 5D-FIG. 5E show flow cytometry for hypodiploid DNA. FIG. 5F shows that dissipation of ΔΨm was visualized 36 hr after infection using DePsipher. Bar, 100 μm. FIG. 5G shows HGK activates caspases-8 and -3. Cells were assayed 36 hr after infection.

FIG. 6A-FIG. 6F show HGK-induced apoptosis requires the TAK1-JNK death pathway. FIG. 6A-FIG. 6D show activation of JNK by HGK is blocked by kinase-inactive TAK1. Western blotting was performed to detect the activating phosphorylation of terminal MAPKs. FIG. 6E shows ceramide-induced apoptosis is inhibited by kinase-inactive mutations of HGK and TAK1. Left, above, DNA histograms by flow cytometry. Left, below, dissipation of ΔΨm visualized with DePsipher. FIG. 6F shows HGK-induced apoptosis is inhibited by kinase-inactive mutations of TAK1 and JNK1.

FIG. 7A-FIG. 7G shows the HGK-TAK1-TRF2 cycle amplifies apoptotic signals. FIG. 7A and FIG. 7B show TRF2 modulates HGK activity. In FIG. 7A, cardiomyocytes were infected for 24 hr with Flag-HGK and the TRF2 vectors shown. HGK kinase activity was increased by dnTRF2; conversely, basal HGK kinase activity was suppressed by wild-type TRF2. In FIG. 7B, TRF2 and GFP antisense oligos were transfected into mouse cardiomyocytes and infected with HGK adenovirus. HGK kinase activity was increased 1.5 fold by knock down of endogenous TRF2. FIG. 7C shows that apoptosis provoked by telomere dysfunction is reduced by dominant-negative mutations of TAK1 and JNK. Cardiomyocytes were infected for 48 hr with the vectors shown, then were assayed by flow cytometry. FIG. 7D shows that HGK-induced apoptosis is partially rescued by exogenous TRF2 or, more completely, Bcl-2. Cardiomyocytes were infected for 36 hr as shown, then were assayed by flow cytometry. FIG. 7F shows kinase-inactive HGK, kinase-inactive TAK1, and Bcl-2 rescued TRF2 levels in ceramide-treated cells. FIG. 7D-7F show equivalent results. FIG. 7G shows caspase-dependent and caspase-independent loss of TRF2, triggered by HGK and ceramide, respectively.

FIG. 8A-FIG. 8I show HGK (MAP4K4) is activated by and potentiates cardiac death signals. FIGS. 8A and 8B show structure and expression of the conventional (FIG. 8A) and conditional (FIG. 8B) HGK transgenes. Upper rows, PCR; lower rows, Western blot. All subsequent data are from αMHC-HGK line 1998, excepting HGK activation by load, which was tested in conditional (“bigenic”) mice. FIG. 8C shows HGK activation by ischemia/reperfusion (30 min/2 hr; left), load (transverse aortic constriction, 14 d; middle), αMHC-TNFα (right), and αMHC-Gq (right). Upper row, immune complex kinase assays; lower row, Western blots. FIG. 8D-8H show HGK provokes a lethal apoptotic cardiomyopathy in concert with Gq. FIG. 8D shows Anatomy (top), hematoxylin-eosin stain (middle), and picrosirius red stain (bottom). Bar, 1 mm (top, middle); 100 μm (bottom). FIG. 8E shows a TUNEL stain. FIG. 8F shows caspase-3 cleavage (left). Upper rows, PCR; lower rows, Western blot. JNK and P38 activation (right). FIG. 8G shows survival. FIG. 8H shows that HGK potentiates Gq-induced apoptosis, shown by flow cytometry (as in FIG. 8E). FIG. 8I shows doppler-echocardiography showing decreased peak aortic ejection velocity, a measure of ventricular systolic performance.

FIG. 9 shows a proposed model for HGK activation and function in cardiomyocyte survival.

### DETAILED DESCRIPTION OF THE INVENTION

The present invention relates to methods of administering compositions of modulators that regulate telomere repeat-binding factor, TRF2, and checkpoint kinase 2, Chk2, in order to treat cardiovascular disease as caused by loss of cardiomyocyte due to apoptosis.

It is readily apparent to one skilled in the art that various embodiments and modifications can be made to the invention disclosed in this Application without departing from the scope and spirit of the invention.

### I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

As used herein, the term “activator” or “effector” refers to a compound that enhances or increases activity. It is envisioned that the “activator” or “effector” can activate activity at any point along a pathway, for example, but not limited to increasing association of TRF2 with the telomere.

The term “apoptosis” is defined as a genetically determined destruction of cells from within due to activation of a stimulus or removal of a suppressing agent or stimulus that is postulated to exist to explain the orderly elimination of superfluous cells. To one skilled in the art the term “apoptosis” is also often referred to as programmed cell death.

As used herein, the term “cardiovascular disease or disorder” refers to disease and disorders related to the cardiovascular or circulatory system. Cardiovascular disease and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., hypertension, arteriosclerosis, aneurysm) or veins (i.e., varicose veins, hemorrhoids). Yet further, one skilled in the art recognizes that cardiovascular diseases and/or disorders can result from congenital defects, genetic defects, environmental influences (i.e., dietary influences, lifestyle, stress, etc.), and other defects or influences.

As used herein, the terms “effective amount” or “therapeutically effective amount” refers to an amount that results in an improvement or remediation of the symptoms of the disease or condition.

As used herein, the term “DNA” is defined as deoxyribonucleic acid.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat heart disease, thus the expression construct or transgene is a therapeutic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other fluctions as well and are described infra.

As used herein, the term “gene” is defined as a functional protein, polypeptide, or peptide-encoding unit. As will be understood by those in the art, this functional term includes genomic sequences, cDNA sequences, and smaller engineered gene segments that express, or is adapted to express, proteins, polypeptides, domains, peptides, fusion proteins, and mutants.

As used herein, the term “heart failure” refers to the loss of cardiomyocytes such that progressive cardiomyocyte loss over time leads to the development of a pathophysiological state whereby the heart is unable to pump blood at a rate commensurate with the requirements of the metabolizing tissues or can do so only from an elevated filling pressure. The cardiomyocyte loss leading to heart failure may be caused by apoptotic mechanisms.

As used herein, the term “heterologous” is defined as DNA or RNA sequences or proteins that are derived from different species.

As used herein, the term “homologous” is defined as DNA or RNA sequences or proteins that are derived from the same species.

As used herein, the term “ischemic heart disease” refers to a lack of oxygen due to inadequate perfusion or blood supply. Ischemic heart disease is a condition having diverse etiologies. One specific etiology of ischemic heart disease is the consequence of atherosclerosis of the coronary arteries.

As used herein, the term “inhibitor” refers to a compound that inhibits or blunts activity. It is envisioned that the “inhibitor” can inhibit activity at any point along a pathway, for example, but not limited to prohibiting phosphorylation of Chk2 and/or inhibiting HGK activity.

As used herein, the term “infarct” or “myocardial infarction (MI)” refers to an interruption in blood flow to the myocardium. Thus, one of skill in the art refers to MI as death of cardiac muscle cells resulting from inadequate blood supply.

As used herein, the term “myocardium” refers to the muscle of the heart.

As used herein, the term “modulator” refers to a compound that either inhibits or enhances TRF2 or Chk2 activity. For example, the modulator increases or enhances TRF2 activity or inhibits or blunts Chk2 activity. It is envisioned that the modulator regulates and/or maintains telomere stability. The modulator of TRF2 may also be referred to as an “activator” or “effector” of TRF2 that can effect or regulate activity of TRF2 or expression of TRF2 at any point along a pathway, for example, but not limited to increasing association of TRF2 with the telomere. The modulator of Chk2 may also be referred to as an “inhibitor” that can inhibit activity Chk2 and/or expression of Chk2 at any point along a pathway, for example, but not limited to prohibiting phosphorylation of Chk2. Thus, one of skill in the art recognizes that the modulators of the present invention maintain or regulate telomere stability at any point along the known pathway, or yet undiscovered pathway, including but not limiting to telomeric signaling, association of proteins with telomeres, increasing expression and/or activity of enzymes, decreasing expression and/or activity of known inhibitors or yet undiscovered inhibitors, increasing expression and/or activity of known activators or yet undiscovered activators, etc.

The term “palliating” a disease as used herein means that the extent or undesirable clinical manifestations of a disease state are lessened and/or the time course of the progression is slowed or lengthened, as compared to the disease in the absence of the substance and/or composition of the present invention.

As used herein, the term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCRTM, and the like, and by synthetic means. Furthermore, one skilled in the art is cognizant that polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “subject” may encompass any vertebrate including but not limited to humans, mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like

As used herein, the term “telomere stability” refers to the state or quality of the telomere being constant or resistant to change and/or deterioration. Thus, one of skill in the art recognizes that telomere stability encompasses all gene expressions, protein interactions, protein degradations, etc. that play a role in maintaining telomere integrity and/or telomere length.

As used herein, the term “treating” and “treatment” and/or “palliating” refers to administering to a subject an effective amount of a the composition so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Thus, one of skill in the art realizes that a treatment may improve the disease condition, but may not be a complete cure for the disease. As used herein, the term “treatment” includes prophylaxis.

As used herein, the term “RNA” is defined as ribonucleic acid.

As used herein, the term “under transcriptional control” or “operatively linked” is defined as the promoter is in the correct location and orientation in relation to the nucleic acid to control RNA polymerase initiation and expression of the gene.

### II. Telomeres and Telomere Associated Proteins

The telomere is a characteristic sequence found at the end of eukaryotic chromosomes that maintains the length of the chromosome. Human telomeres are composed of long arrays of TTAGGG repeats that form a nucleoprotein complex required for the protection and replication of chromosome ends. With each round of chromosomal replication the chromosome potentially becomes shorter because DNA polymerase is unable to replicate the end of linear DNA molecules. To counteract this, proteins associated with the telomere prevent the loss of genetic material by replicating the telomere in a special way. Telomere length in human cells is controlled by a mechanism that involves several enzymes, mainly telomerase and the negative regulators of telomere length, telomere repeat binding factors 1 and 2, TRF1 and TRF2.

The telomere consists of protein-DNA complexes. One major component of telomere maintenance is telomerase. Telomerase is an enzyme that recognizes guanine rich sequences on telomeres and elongates the telomere in the 5′ to 3′ direction by adding hexameric repeats of 5′-TTAGGG-3′ to the ends of eukaryotic chromosomal DNA. Telomerases contain an essential RNA subunit (TER), as well as an essential protein reverse transcriptase subunit (TERT). A special component of telomerase is a built-in RNA template (TER) that the enzyme utilizes to elongate telomeres in the absence of complementary DNA sequences. Telomerase extends chromosome ends by iterative reverse transcription of TER. Following the addition of each telomeric repeat, the RNA template and the telomeric substrate reset their relative position in the active site provided by TERT. DNA replication is completed after telomerase has carried out several rounds of telomere replication. Telomerase has also been implicated in cellular immortalization and cellular senescence.

Telomerase is one of many enzymes involved in the maintenance of chromosomal ends. TTAGGG repeat arrays at the ends of human and mouse chromosomes are also bound by two related proteins. One component of human telomere protein-DNA complex is the telomere repeat binding factor 1 (TRF1), which is present at telomeres throughout the cell cycle. TRF1 is thought to be a telomerase inhibitor by acting in cis to limit the elongation of individual chromosome ends. Another protein found at the telomere is TRF2, a distant homologue of TRF1 that carries a very similar Myb-related DNA-binding motif. Both TRF1 and TRF2 are ubiquitously expressed, bind specifically to duplex TTAGGG repeats in vitro, related to the protooncogene Myb, have dimerization domains near their N terminus, and localize to all human telomeres in metaphase chromosomes. There are significant differences between these two proteins. For example, the dimerization domains of TRF1 and TRF2 do not interact. This suggests that these proteins exist predominantly as homodimers. Although TRF1 and TRF2 have similar telomere binding activity and domain organization, TRF2 has a basic N-terminus and TRF1 has an acidic N-termrinus. Finally, TRF1 is much less conserved than TRF2.

TRF2 may also be involved in negative regulation of telomere length. Indirect immunofluorescence has indicated that both TRF1 and TRF2 may play a role in measuring telomere length by binding to duplex telomeric DNA, especially on telomeres with long TTAGGG repeat tracts. Telomerase expression levels are not affected by either TRF1 or TRF2. Furthermore, enzymatic activity of telomerase in vitro is not affected by the presence of TRF1 or TRF2 on a short linear telomerase. Therefore, sequestration of the 3′ telomere terminus by TRF1- and TRF2-induced telomeric loops may control telomere length by blocking telomerase-dependent telomere elongation.

TRF2 is also implicated in regulating apoptosis. Although broken chromosomes can induce apoptosis, telomeres do not trigger this response. It has been shown that telomeric-repeat binding factor 2 may suppress apoptosis. Proof of this comes from inhibition of TRF2, which results in apoptosis in a subset of mammalian cell types. The TRF2 mediated apoptotic response involves p53 and the ATM (ataxia telangiectasia mutated) kinase, consistent with activation of a DNA damage checkpoint. Telomeres lacking TRF2 may directly signal apoptosis because apoptosis does not occur due to rupture of dicentric chromosomes formed by end-to-end fusion. Telomeres lacking TRF2 possibly resemble damaged DNA. In some cells, lack of TRF2 may signal apoptosis rather than senescence.

### III. Apoptosis

Apoptosis, also known as programmed cell death, is characterized by several changes to the cell, including nuclear chromatin condensation, cytoplasmic shrinking, dilated endoplasmic reticulum, and membrane blebbing. Mitochondria remain morphologically unchanged. Rapid phagocytosis by macrophages makes this type of cell death hard to observe in vivo.

Apoptotic death can be triggered by several stimuli, and not all cells necessarily will respond to the same stimulus. DNA damage (by irradiation or drugs used for cancer chemotherapy), which in many cells leads to apoptotic death via a pathway dependent on p53, is the most studied apoptosis stimuli. Some stimuli, such as corticosteroids, lead to death in particular cells (e.g., thymocytes), but stimulates other cell types. Fas, a surface protein which initiates an intracellular death signal in response to crosslinking is expressed in some cells types. Some cells appear to have a default death pathway that must be actively blocked by a survival factor to allow cell survival.

DNA fragmentation is the first and most dramatic morphological feature in cells undergoing apoptosis. Repeats approximately 200 bp in length are observed when DNA from apoptotically dying cells is subjected to agarose gel electrophoresis. DNA fragmentation can be regarded as a biochemical definition of death because even a few double stranded DNA breaks will render the cell unable to undergo mitosis successfully. The nucleus, however, is not always necessary for apoptotic cell death. In has been shown in some apoptotic systems (e.g., Fas killing of tumor cells) that cells that have their nucleus removed still die.

Macrophages appear to recognize apoptotic cells through several different recognition systems, which seem to be used preferentially by different macrophage subpopulations. There is good evidence that apoptotic cells lose the normal phospholipid asymmetry in their plasma membrane, as manifested by the exposure of normally inward-facing phosphatidyl serine on the external face of the bilayer. Macrophages can recognize this exposed lipid headgroup via an unknown receptor, triggering phagocytosis.

Caspases are another molecular hallmark of programmed cell death. An inactive proenzyme form of caspases seem to be widely expressed by most cells. Active caspases can often initiation a protease cascade. Several protein substrates have been shown to be cleaved by caspases during apoptotic death, yet the functionally important substrates are not known. The most convincing evidence that these proteases are involved in programmed cell death has come from the ability of specific caspase inhibitors to block apoptosis. Also, knockout mice lacking caspase 3, 8 and 9 fail to complete normal embryonic development.

### IV. Cell Cycle Checkpoints

In order for cells to grow and divide they must progress through an orderly sequence of events that results in the duplication of cellular content and ultimately division into two cells. In other words, the cell cycle is a collection of highly ordered processes that result in the duplication of a cell. As cells progress through the cell cycle, they undergo several discrete transitions. A cell cycle transition is defined as a unidirectional change of state in which a cell that was performing one set of processes shifts its activity to perform a different set of processes. The cell cycle consists of four phases, G1 (Growth phase 1), S (Synthesis), G2 (Growth phase 2), and M (Mitosis).

Throughout the eukaryotic cell-division cycle are points at which the cell cycle can be halted until conditions are suitable for the cell to proceed to the next stage. These point are known as cell cycle checkpoints. Cell cycle checkpoints are regulatory pathways that control the order and timing of cell cycle transitions and ensure that critical events such as DNA replication and chromosome segregation are completed with high fidelity. A checkpoint can also be described as a biochemical pathway that ensures dependence of one process upon another process that is otherwise biochemically unrelated. In addition, checkpoints respond to damage by arresting the cell cycle to provide time for repair and by inducing transcription of genes that facilitate repair.

There are four major types of checkpoints that control the progression of the cell cycle from one phase to the next. First, the G1 check point controls the progression of the cell cycle from the G1 phase to the S phase. Here the cell size and a favorable environment are first determined. Second, the DNA damage checkpoints ensure that the DNA is suitable for replication. Several DNA damage checkpoints exist. One well understood DNA damage checkpoint is the G1 DNA damage checkpoint, where the integrity of the DNA is inspected prior to its replication. If DNA is not in proper order, than the cell will likely undergo apoptosis. There is also an S-phase checkpoint that slows DNA replication down to allow for DNA repair. The third type of checkpoint is the G2 checkpoint (also known as the S-M checkpoint). This checkpoint ensures that all the DNA is replicated properly and only one time before progressing to mitosis. Also, DNA damage may be repaired at this checkpoint. Finally the Metaphase checkpoint tracks the alignment of the chromosome on the spindles during mitosis.

The enzymatic machinery involved in cell cycle progression consists of two major types of proteins, the cyclin-dependant kinases, or cdk's, and the cyclins. Kinases, in general, are a group of enzymes involved in the phosphorylation of substrates. Protein kinases specifically phosphorylate serine, threonine, or tyrosine residues on other proteins. Cyclin-dependant kinases rely on cyclins for substrate specificity. Cyclins themselves are produced and degraded with every cell cycle, hence the name cyclins. The activation or inactivation by cyclins of cdk's is what marks the transition through the cell cycle.

Cell cycle checkpoint kinases control the progression of the cell cycle by phosphorylating key components of a signaling pathway, which results in activation or inhibition of that component. Checkpoint pathways consist of three parts: sensors of DNA damage, transducers that relay that there is DNA damage, and effectors that activate the means for repairing the DNA damage. Two major DNA damage checkpoint pathway transducers are ATM (Ataxia-Telangiectasia Mutated) and ATR (Ataxia-Telangiectasia and Rad3-related) kinases. There are several ways in which these kinases regulate the progression of the cell cycle in the presence of irregular DNA. Most of there activity is perpetuated through another key cell cycle protein, p53. ATM and ATR either directly phosphorylate p53, phosphorylate the p53 inhibitor Mdm2, or phosphorylate the checkpoint kinase Chk2. All three increase the activity of p53 resulting in either DNA damage repair or apoptosis. Phosphorylation of p53 or Mdm2 reduces the interaction between these two proteins. Mdm2 targets p53 for degradation. Phosphorylation of Chk2 by ATM or ATR increases its ability to phosphorylate p53. Increased abundance of p53 leads to cell death. Both TRF2 and Chk2 are associated with apoptosis through ATM and p53. Cells lacking Chk2 show reduce accumulation of p53 in response to DNA damage. Cells lacking TRF2 have an increase in Chk2 activation. Both enzymes can therefore be manipulated to regulate apoptosis in cardiomyocytes.

### V. Modulators

In certain embodiments, modulators of TRF2 are administered to a subject to enhance the activity and/or expression of TRF2. Yet further modulators of Chk2 are administered to a subject to suppress the activity and/or expression of Chk2. It is envisioned that TRF2 and/or Chk2 plays a role in telomere stability in cardiomyocytes. In specific embodiments, inhibition of Chk2 attenuates apoptosis of cardiomyocytes.

The modulators of the present invention include, but are not limited to polynucleotides, polypeptides, antibodies, small molecules or other compositions that are capable of modulating either the activity and/or the expression of TRF2 or Chk2.

In specific embodiments of the present invention modulators TRF2 may comprise modulators of apoptosis, for example, but not limited to mitogen-activated protein kinases (MAPKs), more specifically, a MAP kinase kinase kinases (MAP3Ks) or MAP kinase kinase kinase kinases (MAP4Ks). Among the MAP3Ks, transforming growth factor β-activated kinase-1 (TAK1, MAP3K7). TAK1-binding protein-i (TAB1) binds TAK1, induces TAK1 autophosphorylation, and couples TAK1 to p38 and JNK (Shibuya et al., 1996; Kishimoto et al., 2000; and Ono et al., 2001). In addition, the Ste20-like kinase hematopoietic progenitor kinase/germinal center kinase-like kinase (HGK, MAP4K4) activates TAK1, but couples it specifically to JNK (Yao et aL., 1999). Ste 20-like kinases exist as two subfamilies, the p21-activated kinases (PAKs) and germinal center kinase (GCKs), which lack the Rac/Cdc42-binding domain (Dan et al, 2001; and Manning et al., 2002).

Thus, in certain embodiments of the present invention, it is contemplated that modulators, more specifically, inhibitors of HGK, HGK-activated kinases and/or HGK-related kinases are modulators of TRF2. More specifically, an inhibitor of HGK increases the expression and/or activity of TRF2 thereby modulating telomere loss and/or dysfunction. Examples of HGK-activated kinases include, but are not limited to TAK1, or JNK1. Additional examples of HGK-related kinases include Ste-20-like kinases. Thus, the present invention encompasses other Ste-20-like kinases of which a complete description of Ste-20 like kinases can be found in U.S. Pat. Nos. 6,680,170 and 6,569,658 which are both incorporated by reference herein in their entirety.

Still further, other compositions of TRF2 modulators include, but are not limited to compositions discussed in U.S. application Ser. No. 20020076719 or U.S. Pat. No. 6,297,356, which are incorporated herein by reference. Yet further, modulator compositions of Chk2 can include, but are not limited to compositions discussed in U.S. Pat. No. 6,451,538, which is incorporated herein by reference.

In this patent, the terms “TRF2 gene product”; “Chk2 gene product”; “HGK gene product”; “TAK1 gene product” or “JNK1 gene product” refer to proteins and polypeptides having amino acid sequences that are substantially identical to the native TRF2, Chk2, HGK, TAK1 and/or JNK1 amino acid sequences (or RNA, if applicable) or that are biologically active, in that they are capable of performing functional activities similar to an endogenous TRF2, Chk2, HGK, TAK1 and/or JNK1 and/or cross-reacting with anti-TRF2 antibody raised against TRF2 and/or cross-reacting with anti-Chk2 antibody raised against Chk2, and/or cross-reacting with anti-HGK, and/or cross-reacting with anti-TAK1 antibody raised against TAK1; and/or cross-reacting with anti-JNK1 antibody raised against JNK1.

The terms “TRF2 gene product or Chk2 gene product or HGK gene product or TAK1 gene product or JNK1 gene product” also include analogs of the respective molecules that exhibit at least some biological activity in common with their native counterparts. Such analogs include, but are not limited to, truncated polypeptides and polypeptides having fewer amino acids than the native polypeptide. The TRF2 polypeptide sequences include, but are not limited to SEQ.ID.NO.1 (GenBank accession # NP_{—}005643). Chk2 polypeptide sequences include, but are not limited to SEQ.ID.NO.2 (GenBank accession # NP_{—}009125) or SEQ.ID.NO.3 (GenBank accession # NP_{—}665861). HGK polypeptide sequences include, but are not limited to SEQ.ID.NO.4 (GenBank accession # P97820), SEQ.ID.NO.5 (GenBank accession # 095819), SEQ.ID.NO.6 (GenBank accession # NP_{—}663720), SEQ.ID.NO.7 (GenBank accession # NP_{—}663719), SEQ.ID.NO.8 (GenBank accession # NP_{—}004825) and SEQ.ID.NO.9 (GenBank accession # AA032626). TAK1 polypeptide sequences include, but are not limited to SEQ.ID.NO.10 (GenBank accession # NP_{—}006107). JNK1 polypeptide sequences include, but are not limited to SEQ.ID.NO.11 (GenBank accession # NP_{—}620637).

The term “TRF2 gene” “TRF2 polynucleotide” or “TRF2 nucleic acid” refers to any DNA sequence that is substantially identical to a DNA sequence encoding an TRF2 gene product as defined above. Similar terms for HGK and/or Chk2 and/or TAK1 and/or JNK1 are within the scope of the present invention. The term also refers to RNA or antisense sequences compatible with such DNA sequences. An “TRF2 gene or TRF2 polynucleotide” may also comprise any combination of associated control sequences. The TRF2 polynucleotide sequences include, but are not limited to SEQ.ID.NO.12 (GenBank accession # NM_{—}005652). Chk2 polynucleotide sequences include, but are not limited to SEQ.ID.NO.13 (GenBank accession # NM_{—}007194) or SEQ.ID.NO.14 (GenBank accession # NM_{—}145862). HGK polynucleotide sequences include, but are not limited to SEQ.ID.NO.15 (GenBank accession # NM_{—}145687), SEQ.ID.NO.16 (GenBank accession #NM_{—}145686), SEQ.ID.NO.17 (GenBank accession # NM_{—}004834), or SEQ.ID.NO.18 (GenBank accession # AY212247). TAK1 polynucleotide sequences include, but are not limited to SEQ.ID.NO.19 (GenBank accession # NM_{—}006116) and JNK1 polynucleotide sequences include, but are not limited to SEQ.ID.NO.20 GenBank accession # NM_{—}139049).

Thus, nucleic acid compositions encoding TRF2, Chk2, HGK, HGK-related kinases, and/or HGK-activated kinases (i.e., TAK1 and/or JNK1) are herein provided and are also available to a skilled artisan at accessible databases, including the National Center for Biotechnology Information's GenBank database and/or comrnmercially available databases, such as from Celera Genomics, Inc. (Rockville, Md.). Also included are splice variants that encode different forms of the protein, if applicable. The nucleic acid sequences may be naturally occurring or synthetic.

As used herein, the terms “TRF2 and/or Chk2 and/or HGK and/or TAK1 and/or JNK1 nucleic acid sequence,” “TRF2 and/or Chk2 and/or HGK and/or TAK1 and/or JNK1 polynucleotide,” and “TRF2 and/or Chk2 and/or HGK and/or TAK1 and/or JNK1 gene” refer to nucleic acids provided herein, homologs thereof, and sequences having substantial similarity and function, respectively. A skilled artisan recognizes that the sequences are within the scope of the present invention if they encode a product which regulates at least one of the following functions, telomere stability, telomere length, telomere signaling, or apoptosis, and furthermore knows how to obtain such sequences, as is standard in the art.

The term “substantially identical”, when used to define either a TRF2 and/or Chk2 and/or HGK and/or TAK1 and/or JNK1 amino acid sequence or TRF2 and/or Chk2 and/or HGK and/or TAK1 and/or JNK1 polynucleotide sequence, means that a particular subject sequence, for example, a mutant sequence, varies from the sequence of natural TRF2 and/or Chk2 and/or HGK and/or TAK1 and/or JNK1, respectively, by one or more substitutions, deletions, or additions, the net effect of which is to retain at least some of the biological activity found in the native TRF2 and/or Chk2 and/or HGK and/or TAK1 and/or JNK1 protein, respectively. Alternatively, DNA analog sequences are “substantially identical” to specific DNA sequences disclosed herein if: (a) the DNA analog sequence is derived from coding regions of the natural TRF2 and/or Chk2 and/or HGK and/or TAK1 and/or JNK1 gene, respectively; or (b) the DNA analog sequence is capable of hybridization to DNA sequences of TRF2 and/or Chk2 and/or HGK and/or TAK1 and/or JNK1 under moderately stringent conditions and TRF2 and/or Chk2 and/or HGK and/or TAK1 and/or JNK1, respectively having biological activity similar to the native proteins; or (c) DNA sequences which are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s)” or “moderately stringent conditions”.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

A. Expression Vectors

The present invention can involve using expression constructs as the pharmaceutical compositions. It is contemplated that the expression construct comprises polynucleotide sequences encoding polypeptides which can act as modulators of telomere stability. Such expression constructs include, but are not limited to constructs containing an inhibitor of Chk2 expression or an inhibitor of HGK expression or inhibitor of TAK1 expression or inhibitor of JNK1, or an activator of TRF2 expression. It is contemplated that the inhibitor of Chk2 modulates or suppresses apoptotic signaling. In specific embodiments, the inhibitor suppresses transcription of a chk2, hgk, tak1 and/or jnk1 gene. It is further contemplated that the activator of TRF2 stimulates or enhances TRF2 expression resulting in an increase in telomere stability and a decrease apoptosis. The activator of TRF2 can be a compound that enhances transcription of a trf2 gene. Still further, other modulators of telomere stability include compounds that enhance TRF2, for example inhibitors that regulate, decrease, or inhibit HGK functional activity or expression.

In certain embodiments, the present invention involves the manipulation of genetic material to produce expression constructs that encode inhibitors of Chk2, inhibitors of HGK, inhibitors of TAK1 or inhibitors of JNK1 or activators of TRF2. Thus, the inhibitor or activator is contained in an expression vector. Such methods involve the generation of expression constructs containing, for example, a heterologous nucleic acid sequence encoding an inhibitor or activator of interest and a means for its expression, replicating the vector in an appropriate cell, obtaining viral particles produced therefrom, and infecting cells with the recombinant virus particles.

In one embodiment, a gene encoding a TRF2 or structural/functional domain thereof is introduced in vivo in a viral vector. Such vectors include an attenuated or defective DNA virus, such as but not limited to herpes simplex virus (HSV), papilloma virus, Epstein Barr virus (EBV), adenovirus, adeno-associated virus (AAV), lentivirus and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al., 1991) an attenuated adenovirus vector, (Stratford-Perricaudet et al., 1992), and a defective adeno-associated virus vector (Samulski et al., 1987 and Samulski et al., 1989).

Preferably, for in vitro administration, an appropriate immunosuppressive treatment is employed in conjunction with the viral vector, e.g., adenovirus vector, to avoid immunodeactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (Wilson, Nature Medicine (1995). In addition, it is advantageous to employ a viral vector that is engineered to express a minimal number of antigens.

In another embodiment the gene can be introduced in a retroviral vector, e.g., as described in Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995, by Dougherty et al.; and Kuo et al., Blood, 82:845 (1993). Targeted gene delivery is described in International Patent Publication WO 95/28494, published October 1995.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al., 1987; Mackey et al., 1988). The use of cationic lipids may promote encapsulation of negatively charged nucleic acids, and also promote fusion with negatively charged cell membranes (Felgner and Ringold, 1989). The use of lipofection to introduce exogenous genes into the specific organs in vivo has certain practical advantages. Molecular targeting of liposomes to specific cells represents one area of benefit. Lipids may be chemically coupled to other molecules for the purpose of targeting. Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (Wu et al., 1992; Wu and Wu, 1988; Hartmut et al., Canadian Patent Application No. 2,012,311, filed Mar. 15, 1990).

A gene therapy vector as described above can employ a transcription control sequence operably associated with the sequence for the TRF2 inserted in the vector. Such an expression vector is particularly useful to regulate expression of a therapeutic TRF2 gene. In one embodiment, the present invention contemplates constitutive expression of the TRF2 gene, even if at low levels.

B. Transcription Factors and Nuclear Binding Sites

Transcription factors are regulatory proteins that binds to a specific DNA sequence (e.g., promoters and enhancers) and regulate transcription of an encoding DNA region. Typically, a transcription factor comprises a binding domain that binds to DNA (a DNA binding domain) and a regulatory domain that controls transcription. Where a regulatory domain activates transcription, that regulatory domain is designated an activation domain. Where that regulatory domain inhibits transcription, that regulatory domain is designated a repression domain.

Activation domains, and more recently repression domains, have been demonstrated to function as independent, modular components of transcription factors. Activation domains are not typified by a single consensus sequence but instead fall into several discrete classes: for example, acidic domains in GAL4 (Ma, et al. 1987), GCN4 (Hope, et al., 1987), VP16 (Sadowski, et al. 1988), and GATA-1 (Martin, et al. 1990); glutamine-rich stretches in Sp1 (Courey, et al. 1988) and Oct-2/OTF2 (Muller-lmmergluck, et al. 1990; Gerster, et al. 1990); proline-rich sequences in CTF/NF-1 (Mermod, et al. 1989); and serine/threonine-rich regions in Pit-1/GH-F-1 (Theill, et al. 1989) all function to activate transcription. The activation domains of fos and jun are rich in both acidic and proline residues (Abate, et al. 1991; Bohmann, et al. 1989); for other activators, like the CCAAT/enhancer-binding protein C/EBP (Friedman, et al. 1990), no evident sequence motif has emerged.

In the present invention, it is contemplated that transcription factors can be used to inhibit the expression of a chk2 gene, hgk, tak1, jnk1 and/or enhance or activate the expression of trf2 gene.

C. Antisense and Ribozymes

An antisense molecule that binds to a translational or transcriptional start site, or splice junctions, are ideal inhibitors. Antisense, ribozyme, and double-stranded RNA molecules target a particular sequence to achieve a reduction or elimination of a particular polypeptide, such as Chk2, HGK, TAK1 and/or JNK1, other HGK-related kinases or HGK-activated kinases. Thus, it is contemplated that antisense, ribozyme, and double-stranded RNA, and RNA interference molecules are constructed and used to inhibit Chk2, HGK, TAK1, and/or JNK1 expression.

1. Antisense Molecules

Antisense methodology takes advantage of the fact that nucleic acids tend to pair with complementary sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Antisense constructs are designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction are used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

It is advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

2. Ribozymes

Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression is particularly suited to therapeutic applications (Scanlon et al., 1991; Sarver et al., 1990; Sioud et al., 1992). Most of this work involved the modification of a target mRNA, based on a specific mutant codon that is cleaved by a specific ribozymne. In light of the information included herein and the knowledge of one of ordinary skill in the art, the preparation and use of additional ribozymes that are specifically targeted to a given gene will now be straightforward.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis 6 virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in Chk2 targeted ribozymes is simply a matter of preparing and testing a given sequence, and is a routinely practiced screening method known to those of skill in the art.

3. RNA Interference

It is also contemplated in the present invention that double-stranded RNA is used as an interference molecule, e.g., RNA interference (RNAi). RNA interference is used to “knock down” or inhibit a particular gene of interest by simply injecting, bathing or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000).

Thus, in certain embodiments, double-stranded Chk2, HGK, TAK1, JNK1, HGK-activated kinase, or related-HGK kinase RNA is synthesized or produced using standard molecular techniques well known and used by those of skill in the art.

D. Protein Variants

Amino acid sequence variants of the TRF2, Chk2, HGK, TAK1, JNK1, HGK-activated kinases, and/or HGK-related kinases proteins can be used as modulators of TRF2 and/or Chk2. These variants can be substitutional, insertional or deletion variants. These variants may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

It is thus contemplated by the inventors that various changes may be made in the DNA sequences of genes without appreciable loss of their biological utility or activity, as discussed below. The activity being telomere signaling, telomere stability, etc.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within +1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

1. Fusion Proteins

A specialized kind of insertional variant is the fusion protein. This molecule generally has all or a substantial portion of the native molecule, linked at the N- or C-terminus, to all or a portion of a second polypeptide. For example, a fusion protein of the present invention can includes the addition of a protein transduction domains, for example, but not limited to Antennepedia transduction domain (ANTP), HSV1 (VP22) and HIV-1(Tat). Fusion proteins containing protein transduction domains (PTDs) can traverse biological membranes efficiently, thus delivering the protein of interest (TRF2 and/or Chk2 and/or HGK, TAK1, or JNK1 or variants thereof) into the cell. (Tremblay, 2001; Forman et al., 2003).

Yet further, inclusion of a cleavage site at or near the fusion junction will facilitate removal of the extraneous polypeptide after purification. Other useful fusions include linking of functional domains, such as active sites from enzymes, glycosylation domains, other cellular targeting signals or transmembrane regions.

2. Domain Switching

An interesting series of variants can be created by substituting homologous regions of various proteins. This is known, in certain contexts, as “domain switching.”

Domain switching involves the generation of chimeric molecules using different but, in this case, related polypeptides. By comparing various TRF2 and/or Chk2 proteins, one can make predictions as to the functionally significant regions of these molecules. It is possible, then, to switch related domains of these molecules in an effort to determine the criticality of these regions to function of the protein. These molecules may have additional value in that these “chimeras” can be distinguished from natural molecules, while possibly providing the same function.

3. Synthetic Peptides

The present invention also describes smaller TRF2-related peptides or Chk2-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### VI. Screening for Modulators

The present invention comprises methods for identifying modulators that affect the function of telomere repeat-binding factor 2 (TRF2) and checkpoint kinase 2 (Chk2). These assays may comprise random screening of large libraries of candidate substances; alternatively, the assays may be used to focus on particular classes of compounds selected with an eye towards structural attributes that are believed to make them more likely to modulate the function or activity of TRF2 or Chk2

By function, it is meant that one may assay for mRNA expression, protein expression, protein activity, telomere binding activity, or ability to associate and/or dissociate from other members of the complex and otherwise determine functions contingent on the TRF2 and/or Chk2 proteins.

A. Modulators and Assay Formats

The present invention provides methods of screening for modulators of TRF2 activity, e.g., activity of TRF2 and/or expression of TRF2 proteins or nucleic acids, or modulators of Chk2 activity, e.g., activity of Chk2 and/or expression of Chk2 proteins or nucleic acids.

In certain embodiments, screening for modulators of TRF2 activity may also comprise screening for modulators of HGK, HGK-activated kinases (i.e., TAK1 and/or JNK1), HGK-related kinases.

1. Assay Formats

In one embodiment, the present invention is directed to a method of: obtaining TRF2 and/or Chk2 and/or HGK; contacting the TRF2 and/or Chk2 and/or HGK with a candidate substance; and assaying for TRF2 and/or Chk2 and/or HGK activity. The difference between the measured activity with and without the candidate substance indicates that said candidate substance is, indeed, a modulator of the TRF2 and/or Chk2 and/or HGK activity. Assays may be conducted in cell free systems, in isolated cells, or in organisms including transgenic animals.

2. Inhibitors

An inhibitor according to the present invention may be one which exerts an inhibitory effect on the expression, activity or function of Chk2. The inhibitor may inhibit Chk2 anywhere along its pathway. Other inhibitors may also include inhibitors of HGK, HGK-activated kinases (i.e., TAK1 and/or JNK1), HGK-related kinases.

3. Activators

An activator according to the present invention may be one which exerts a positive or stimulatory effect on the expression, activity or function of TRF2. It is envisioned that the “activator” or “effector” can activate TRF2 at any point along a pathway, for example, but not limited to increasing association of TRF2 with the telomere. Since inhibition of the HGK activation pathway can result in an increase in expression, activity or function of TRF2, an activator of TRF2 may also comprise inhibitors of HGK, HGK-activated kinases and/or HGK-related kinases.

4. Candidate Substance

As used herein, the term “candidate substance” refers to any molecule that may potentially modulate TRF2 or Chk2 or HGK activity, expression or function. Candidate compounds may include fragments or parts of naturally-occurring compounds or may be found as active combinations of known compounds which are otherwise inactive. The candidate substance can be a polynucleotide, a polypeptide, a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Schreiber et al., 2001a; Schreiber et al., 2001b). Chemical genetics is the technology that uses small molecules to modulate the fimctions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The most useful pharmacological compounds may be compounds that are structurally related to compounds which interact naturally with enzymes that bind the telomere. Creating and examining the action of such molecules is known as “rational drug design,” and include making predictions relating to the structure of target molecules. Thus, it is understood that the candidate substance identified by the present invention may be a small molecule activator or any other compound (e.g., polypeptide or polynucleotide) that may be designed through rational drug design starting from known activators of telomere binding proteins.

The goal of rational drug design is to produce structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule like telomere binding protein, and then design a molecule for its ability to interact with telomere binding protein. This could be accomplished by X-ray crystallography, computer modeling or by a combination of both approaches. The same approach may be applied to identifying interacting molecules of Chk2 or TRF2 or HGK.

It also is possible to use antibodies to ascertain the structure of a target compound or activator. In principle, this approach yields a pharmacore upon which subsequent drug design can be based. It is possible to bypass protein crystallography altogether by generating anti-idiotypic antibodies to a fimctional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is a binding assay. Binding of a molecule to a target (e.g., TRF2 or Chk2 or HGK or HGK-activated kinases or related kinases) may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a TRF2 or Chk2 or HGK or HGK-activated kinases or HGK-related kinases molecules or fragments thereof are provided.

A target telomere associating protein may be either free in solution, fixed to a support, expressed in or on the surface of a cell. Either the target telomere associating protein or the compound may be labeled, thereby indicating if binding has occurred. In another embodiment, the assay may measure the activation of binding of a target telomere associated protein to a natural or artificial substrate or binding partner. Competitive binding assays can be performed in which one of the agents is labeled. Usually, the target telomere associated protein will be the labeled species, decreasing the chance that the labeling will interfere with the binding moiety's function. One may measure the amount of free label versus bound label to determine binding or activation of binding. These approaches may be utilized on cell cycle checkpoint kinases or HGK, HGK-activated kinases or HGK-related kinases.

A technique for high throughput screening of compounds is described in WO 84/03564. Large numbers of small peptide test compounds are synthesized on a solid substrate, such as plastic pins or some other surface. The peptide test compounds are reacted with, for example, telomere associated protein and washed. Bound polypeptide is detected by various methods.

C. In cyto Assays

Various cell lines that express telomere associated proteins can be utilized for screening of candidate substances. For example, cells containing telomere associated proteins with an engineered indicator can be used to study various functional attributes of candidate compounds. In such assays, the compound would be formulated appropriately, given its biochemical nature, and contacted with a target cell. This same approach may utilized to study various functional attributes of candidate compounds that effect cell cycle checkpoint kinases or HGK, HGK-activated kinases or HGK-related kinases.

Depending on the assay, culture may be required. As discussed above, the cell may then be examined by virtue of a number of different physiologic assays (e.g., growth, size, or survival). Alternatively, molecular analysis may be performed in which the function of telomere associated proteins or cell cycle checkpoint kinases and related pathways may be explored. This involves assays such as those for protein production, enzyme function, substrate utilization, mRNA expression (including differential display of whole cell or polyA RNA) and others.

D. In vivo Assays

The present invention particularly contemplates the use of various animal models. For example, transgenic animals may be created with constructs that permit telomere associated protein or cell cycle checkpoint kinase activity to be controlled and monitored. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), and U.S. Pat. No. 6,201,165, the teachings of which are generally known and are incorporated herein.

Treatment of animals with test compounds (e.g., TRF2 or Chk2 or HGK modulators) involve the administration of the compound, in an appropriate form, to the animal. Administration is by any route that could be utilized for clinical or non-clinical purposes, including but not limited to oral, nasal, buccal, or even topical. Alternatively, administration may be by intratracheal instillation, bronchial′ instillation, intradermal, subcutaneous, intramuscular, intraperitoneal or intravenous injection. Specifically contemplated are systemic intravenous injection, regional administration via blood or lymph supply.

E. Production of Modulators

In an extension of any of the previously described screening assays, the present invention also provide for methods of producing modulators, i.e., inhibitors and/or activators. The methods comprising any of the preceding screening steps followed by an additional step of “producing the candidate substance identified as a modulator of” the screened activity.

### VII. Treatment

Embodiments of the present invention relate to methods of enhancing cell survival. The methods comprise modulating the telomere/telomere associated protein complexes and cell cycle checkpoint kinases. More specifically, embodiments of the present invention relate to modulating TRF2 or Chk2 or inhibiting HGK, TAK1 or JNK1 activity to maintain telomere stability, thus decreasing apoptosis and increasing cell survival. Oxidative stress is associated with telomere shortening and/or instability. Yet further, oxidative stress down-regulates TRF2. Thus, the compositions of the present invention modulate the down-regulation of TRF2 resulting in telomere stability and/or cell survival and/or decreased cellular apoptosis. Specific TRF2 modulators can include modulators that effect the activity and/or expression of HGK, HGK-activated kinases and/or HGK-related kinases. More specifically, an inhibitor of HGK enhances TRF2 expression and/or activity thereby promoting telomere stability.

Further, embodiments of the present invention relate to methods of treating cardiovascular disease. The methods comprise modulating the telomere/telomere associated protein complexes and cell cycle checkpoint kinases. More specifically, embodiments of the present invention relate to modulating TRF2 or Chk2 activity to reduce cardiomyocyte apoptosis resulting from stress placed on the heart.

Cardiovascular diseases and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., hypertension, arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), myocardial infarction and ischemic heart disease.

In specific embodiments, the present invention comprises a method of treating a subject suffering from a cardiovascular disease comprising the step of administering to the subject an effective amount of a composition to modulate telomere repeat-binding factor 2 (TRF2) or cell cycle checkpoint kinase 2 (Chk2) activity, wherein the effective amount modulates loss of cardiomyocytes. It is envisioned that the composition is a pharmaceutical composition that comprises a TRF2 activator or Chk2 inhibitor. The TRF2 activator may either enhance the activity and/or expression of TRF2 or it may suppress the down-regulation of TRF2. A particular TRF2 activator is TERT, which prevents or inhibits the down-regulation of TRF2. Another exemplary TRF2 activator is an HGK inhibitor. In further embodiments, the composition comprises a compound that modulates TRF2 activity by prohibiting the suppression of TRF2 may be a composition that inhibits Chk2 activity and/or expression, thus, resulting in blunting or a decrease in apoptosis, i.e., cardiomyocyte loss.

Accordingly, the invention involves the composition of the present invention as a treatment or prevention of any one or more of these conditions or other conditions involving cardiovascular disease, more specifically myocardial infarction and/or heart failure resulting from cardiomyopathy as well as compositions for such treatment or prevention.

Another embodiment is a method of modulating a decrease in cardiac muscle contractile strength in a subject comprising the step of administering to the subject an effective amount of a composition to modulate telomere repeat-binding factor 2 (TRF2) or cell cycle checkpoint kinase 2 (Chk2) activity, wherein the effective amount modulates. cardiac muscle contractile strength.

It is known and understood by those of skill in the art that stroke volume or ventricular work is related to the level of venous inflow, as measured by atrial pressure, or by ventricular end-diastolic volume or end-diastolic pressure. Thus, in a normal heart, the heart will pump whatever volume is brought to it by the venous circulation. The increase in contractile force that occurs in response to ventricular dilation is related to the myofibrillar organization, for example stretching of the sarcomeres. Apoptosis in cardiomyocyte may result from loss of telomere stability. The loss of cardiomyocytes in turn results in the heart having decreased contractile strength resulting in ventricular dysfunction ultimately leading to heart failure. Contractile strength or contractility can be measured by measuring the maximum rate of change in pressure (dp/dt max). Clinically, contractility is measured by ejection fraction. Normally, the heart ejects about 60% of its volume each beat, thus a decrease in the volume is an indicator of decreased contractility or contractile strength and ventricular dysfunction.

Still further, the present invention comprises a method of treating a subject at risk for ventricular dysfunction associated with mechanical stress comprising the step of administering to the subject an effective amount of a composition to modulate telomere repeat-binding factor (TRF2) or cell cycle checkpoint kinase (Chk2) activity, wherein the effective amount decreases ventricular dysfunction.

Another embodiment is a method of regulating cardiomyocyte apoptosis in a subject at risk for heat failure comprising the step of administering to the subject an effective amount of a composition to regulate telomere stability, wherein the effective amount increases cardiomyocyte survival. The composition contains a modulator of TRF2 and/or Chk2.

A further embodiment is a method for regulating telomere stability in cardiomyocytes of a subject at risk for a cardiovascular disease comprising the step of administering to the subject an effective amount of a composition to regulate telomere stability.

Still further, another aspect is a method of regulating oxidative stress in a cardiomyocyte during mechanical stress comprising the steps of administering to the cardiomyocyte a composition to regulate telomere stability resulting in a decrease in oxidative stress in the cardiomyocyte.

Yet further, the methods comprise administering to a subject in need thereof an amount of a substance effective to diminish or reverse progression of the dysfunction. In the context of prophylaxis, a subject in need thereof includes, but is not limited to, individuals in the general population who are 55 years of age and older; individuals who have a genetic predisposition to developing cardiac hypertrophy; dilated cardiac myopathy patients; hypertensive patients; patients with renal failure and vascular hypertension; individuals with vascular hypertensive due to pressure overload, volume overload, or increased peripheral bed resistance; individuals with respiratory ailments such as emphysema or cystic fibrosis; chronic asthmatics; individuals with tuberculosis; and organ transplant patients.

A. Genetic Based Therapies

Specifically, the present inventors intend to provide, to a cell, an expression construct capable of enhancing TRF2 or inhibiting Chk2 or inhibiting HGK, TAK1 or JNK1 to that cell. The discussion of expression vectors and the genetic elements employed therein is incorporated into this section by reference. Particularly preferred expression vectors are viral vectors such as adenovirus, adeno-associated virus, herpes virus, vaccinia virus, lentivirus and retrovirus. Also the vector can be liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient Similar figures may be extrapolated for liposomal or other non-viral formulations by comparing relative uptake efficiencies. Formulation as a pharmaceutically acceptable composition is discussed below.

B. Protein Therapy

Another therapy approach is the provision, to a subject, of TRF2 polypeptide, active fragments, synthetic peptides, mimetics or other analogs thereof. Still further, another therapy approach is the provision, to a subject, of polypeptide, active fragments, synthetic peptides, mimetics or other analogs thereof that result in inhibition of Chk2 or HGK or HGK-related kinases or HGK-activated kinases. The protein may be produced by recombinant expression means. Formulations would be selected based on the route of administration and purpose including, but not limited to, liposomal formulations and classic pharmaceutical preparations.

### VIII. Pharmaceutical Formulations and Treatment Regimens

Where clinical applications are contemplated, it will be necessary to prepare pharmaceutical compositions—expression vectors, polynucleotides, polypeptides, proteins, small molecules and drugs—in a form appropriate for the intended application.

The compositions of the present invention are used to enhance cell survival and/or treat cardiovascular diseases, including, but not limited to, coronary heart disease, arteriosclerosis, ischemic heart disease, angina pectoris, myocardial infarction, congestive heart failure and other diseases of the arteries, arterioles and capillaries or related complaint. Accordingly, the invention involves the administration of composition as a treatment or prevention of any one or more of these conditions or other conditions involving cardiomyopathy, as well as compositions for such treatment or prevention.

The compositions disclosed herein may also include the use of adenovirus (AdV) vectors. These vectors have been used for genetic modification of a variety of somatic cells in vitro and in vivo. They have been widely used as gene delivery vectors in experiments both with curative and preventive purposes. AdV vectors have been used in the experimental and in some extent in the clinical gene therapy of a variety of cancers. In the present invention, AdV vectors would be used to deliver copies of the TRF2 gene to cardiomyocytes to treat cardiovascular disease. AdV vectors may also be utilized to deliver dominant negative gene copies of Chk2, HGK, HGK-related kinases or HGK-activated kinases to help growth and survival of cardiomyocytes. The present invention would also incorporate the combination of recombinant AdV technology with chemotherapy to treat heart failure. In addition to AdV vectors, adeno-associated and lentivirus vectors are also contemplated for use to deliver copies of TRF2 genes to cells to treat disease and/or increase cell survival. Adeno-associated vector have proven useful for gene therapy to treat cardiovascular diseases (Dzau et al., 2002; and Chen et aL., 2002).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fingi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Moreover, for human administration, preparations should meet sterility, pyrogenicity, general safety and purity standards as required by FDA Office of Biologics standards. The formulations are easily administered in a variety of dosage forms such as injectable solutions, drug release capsules and the like.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

It is envisioned one of skill in the art will know the most advantageous routes of administration depending upon the disease. In specific embodiments, it is contemplated that composition can be administered via injection, which includes, but is not limited to subcutaneous, intravenous, intraarterial, intramuscular, intraperitoneal, intramyocardial, transendocardial, transepicardial, intranasal and intrathecal.

In certain aspects, it is envisioned that composition of the present invention can be administered to the subject in an injectable formulation containing any compatible carrier, such as various vehicles, adjuvants, additives, and diluents. Yet further, the composition can be administered parenterally to the subject in the form of slow-release subcutaneous implants or targeted delivery systems such as monoclonal antibodies, iontophoretic, polymer matrices, liposomes, and microspheres.

Treatment regimens may vary as well, and often depend on the cardiovascular disease or disorder, disease progression, and health and age of the subject. Obviously, certain types of cardiovascular disease will require more aggressive treatment, while at the same time, certain patients cannot tolerate more taxing protocols. The clinician will be best suited to make such decisions based on the known efficacy and toxicity (if any) of the therapeutic formulations.

Suitable regimes for initial administration and further doses or for sequential administrations also are variable, and may include an initial administration followed by subsequent administrations; but nonetheless, may be ascertained by the clinician.

For example, the composition of the present invention can be administered initially, and thereafter maintained by further administration. For instance, a composition of the invention can be administered in one type of composition and thereafter further administered in a different or the same type of composition. For example, a composition of the invention can be administered by intravenous injection to bring blood levels to a suitable level. The subject's levels are then maintained by a subcutaneous implant form, although other forms of administration, dependent upon the subject's condition, can be used.

The effective amount is an amount of the composition of the present invention that blunts or reduces cardiomyocyte apoptosis, increase cardiomyocyte cell survival, decreases telomere shortening, loss or dysfunction, increases telomere stability, reduces or minimizes cardiovascular disease, for example, reduces cardiomyopathy associated with heart failure. Thus, an effective amount is an amount sufficient to be detected to and repeatedly ameliorate, reduce, minimize or limit the extent of the disease or its symptoms.

Dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Of course, for any composition to be administered to an animal or human, and for any particular method of administration, it is preferred to determine the toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

The treatments may include various “unit doses.” Unit dose is defined as containing a predetermined-quantity of the composition. The quantity to be administered, and the particular route and formulation, are within the skill of those in the clinical arts. A unit dose need not be administered as a single injection, or capsule, or any other appropriate formulation, but may comprise continuous infusion over a set period of time.

### IX. Combined Treatments

In order to increase the effectiveness of the composition, it may be desirable to combine these compositions and methods of the invention with a known agent effective in the treatment of cardiovascular disease or disorder, for example known agents to treat heart failure. In some embodiments, it is contemplated that a conventional therapy or agent, including but not limited to, a pharmacological therapeutic agent, a surgical therapeutic agent (e.g., a surgical procedure) or a combination thereof, may be combined with the composition of the present invention.

This process may involve contacting the cell(s) with an agent(s) and the composition of the present invention at the same time or within a period of time wherein separate administration of the agent and the composition to a cell, tissue or organism produces a desired therapeutic benefit. The terms “contacted” and “exposed,” when applied to a cell, tissue or organism, are used herein to describe the process by which the composition and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both the composition and one or more agents, or by contacting the cell with two or more distinct compositions or formulations, wherein one composition includes the composition and the other includes one or more agents.

The treatment may precede, be co-current with and/or follow the other agent(s) by intervals ranging from minutes to weeks. In embodiments where the composition, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) with the composition. In other aspects, one or more agents may be administered within of from substantially simultaneously, about minutes to hours to days to weeks and any range derivable therein, prior to and/or after administering the composition.

Administration of the composition to a cell, tissue or organism may follow general protocols for the administration of cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, or a vasopressor. Other drug therapies include treatment agents for congestive heart failure, for example, but not limited to calcium channel blocking agents, β-adrenergic blocking agents, angiotensin II inhibitors or ACE inhibitors. ACE inhibitors include drugs designated by the trademarks Accupril®, Altace®, Capoten®, Lotensin®, Monopril®, Prinivil®, Vasotec®, and Zestril®.

B. Surgical Therapeutic Agents

In certain aspects, a therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### X. EXAMPLES

The following examples are included to demonstrate preferred embodiments of the invention. It should be appreciated by those of skilled in the art that the techniques disclosed in the examples which follow represent techniques discovered by the inventor to fimction well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Patient Samples and Controls

Human myocardium was obtained through the Methodist-DeBakey Heart Center, The Methodist Hospital, Houston, Texas and the Human Heart Tissue Transplant Core, The Cleveland Clinic, Cleveland, Ohio. Tissue procurement was based on patient informed consents and approved by the respective institutional review boards. Heart failure tissue (idiopathic and ischemic dilated cardiomyopathy, DCM) was obtained from explanted hearts at the time of therapeutic transplantation. Normal hearts were obtained from unmatched organ donors and victims of motor vehicle accidents. Hypertrophic obstructive cardiomyopathy (HOCM), a heterogenous primary disorder of heart growth without ventricular pump failure, was also used for comparison.

### Example 2

### Cell Culture and Viral Gene Transfer

Ventricular myocytes from 2 day-old Sprague-Dawley rats were purified and cultured (Oh, H. et al., 2001; Akli, S. et al., 1999); by this age, ventricular myocytes become refractory to serum-induced Gl exit, after initial serum-starvation in vitro (Akli, S. et al., 1999). Plasmids for human TRF1, TRF2, and the corresponding dominant-negative truncations (TRF1DM, TRF2DBDM) were provided by Dr. Titia de Lange (Rockefeller University) (Karlseder, J. et al., 1999). Adenoviruses coexpressing enhanced green fluorescent protein (eGFP) were generated using pAdTrack-cytomegalovirus (CMV) and pShuttle-CMV (provided by Dr. Bert Vogelstein, Johns Hopkins Oncology Center) (Oh, H. et al., 2001; He, T. C., 1998). Myocytes were infected using a multiplicity of infection of 20. To visualize TRF1/2 after gene transfer, myocytes were fixed in 70% ethanol, then incubated sequentially with tetramethyl rhodamine isothiocyanate-conjugated MF-20 antibody to sarcomeric myosin heavy chains to confirm cell type (University of Iowa Hybridoma Bank), rabbit antibodies to TRF1 and TRF2 (#581420 and 581425; 1:500, Calbiochem) and fluorescein isothiocyanate (FITC)-conjugated goat antibody to rabbit IgG (1:1000, Sigma). Nuclei were stained with 4′,6-diamidino-2-phenylindole (DAPI). Images were captured using a Zeiss Axioplan 2 epifluorescence microscope.

### Example 3

### Antisense Oligonucleotides

Three antisense phosphorothioate oligonucleotides for mouse TRF2 were generated (Molecular Research Laboratories), one of which inhibited endogenous TRF2 expression effectively in NIH 3T3 cells (not shown). The sequences used were: antisense TRF2 (asTRF2), 5′-CCTGGGCTGCCGGCTCGAGC-3′ (SEQ ID NO:21); sense TRF2 (sTRF2), 5′-CGAGCTCGGCCGTCGGGTCC-3′ (SEQ ID NO:22), antisense GFP (Sano, M. et al., 2002), 5′-CGTTTACGTCGCCGTCCAGC-3′ (SEQ ID NO:23). Oligonucleotides were transfected into 1-2 day-old C57BI/6 mouse cardiomyocytes, cultured as above, using Oligofectamine (Invitrogen).

### Example 4

### TERT Animal Models

Cardiac-specific TERT transgenic mice (αMHC-TERT) (Oh, H. et al., 2001) and wild-type littermates (10-12 week-old, 18-22 g) were subjected for 1 wk to partial occlusion of the transverse aorta (Zhang, D. et al., 2000). The control “sham” operation comprised anesthesia, thoracotomy, and ligature placement without constriction. The presence and severity of obstruction were corroborated by Doppler flow studies; only mice in which severe load was confirmed (a right to left carotid artery velocity ratio>3.5) were analyzed further. Doppler echocardiography and staining with Sirius red were performed 7 d after surgery (Oh, H. et al., 2001).

### Example 5

### Apoptosis

For myocardium, terminal transferase-mediated dUTP-biotin nick end-labeling (TUNEL) assays were performed using the Oncor ApopTaq Direct in situ Apoptosis detection kit (Zhang, D. et al., 2000), MF20 antibody to sarcomeric MHC, and Texas Red-conjugated antibody to mouse IgG.

Hypodiploid DNA was detected by two-color flow cytometry using propidium iodide for DNA content and FITC-conjugated MF20 (Oh, H. et al., 2001; Akli, S. et al., 1999) or FITC-conjugated antibody to sarcomeric myosin heavy chains to confirm myocyte identity, sampling>5000 myocytes for each histogram.

To detect dissipation of mitochondrial membrane potential (ΔΨm), cells were incubated for 60 min in 5 μg/ml DePsipher (R & D Systems, Minneapolis, Minn.).

To measure caspase-3 and caspase-8 activity, lysates were incubated with 10 nM DEVD-p-nitroaniline (pNA) and 40 nM IETD-pNA (Clontech, Palo Alto, Calif.), respectively, in the presence of 1 mM DTT for 2 hr at 37° C. Substrate cleavage was detected as pNA release using a Beckmann spectrophotometer at 405 nm, calibrated by comparison to known amounts of pNA, and normalized for protein concentration. Full length and cleaved caspase 3 were detected by caspase-3 antibody (H-277; Santa Cruz, Calif.).

### Example 6

### Detection of Telomere Length

DNA was digested with Rsa I, resolved by electrophoresis in 0.5% agarose, transferred to Hybond-N⁺ membranes (Amersham Pharmacia Biotech), and hybridized using a ³²P-labeled (TTAGGG)₄ telomeric probe (Oh, H. et al., 2001; Counter, C. M., 1992). Mean telomere length was ascertained by Phosphor-Imager scanning (Molecular Dynamics).

### Example 7

### Telomerase Expression and Activity

Telomerase activity was measured by a PCR-based telomerase repeat amplification protocol assay using 1 μg of cell or tissue extract (Oh, H. et al., 2001). TERT, the RNA component of telomerase (TERC), and glyceraldehyde-3-phosphate dehydrogenase (GAPDH) were analyzed by RT-PCR in the log-linear range of amplification (Kiaris, H. et al., 1999; Blasco, M. A. et al., 1995; Martin-Rivera, L. et al., 1998).

### Example 8

### Western Blot and Immune Complex Kinase Assays for TRF and Chk2

Proteins were resolved by electrophoresis in 10% SDS-polyacrylamide gels and transferred to membranes by electroblotting. Antibodies were: human and mouse TRF2 (Calbiochem), human and mouse TRF1 (Calbiochem), phospho-Chk2 (Thr68; Cell Signaling), sarcomeric a-actin and myc (Sigmna), FLAG epitope (M2, Kodak), GFP (Clontech), Chk2 (Santa Cruz), poly (ADP-ribose) polymerase (PARP, Oncogene). To detect exogenous TRF2 in virus infected cardiomyocytes, goat and rabbit antibodies to TRF2 were used (C-16, H-300, Santa Cruz); endogenous rat TRF2 was detected using rabbit antibody to TRF2 (Alpha Diagnostic International). After blocking with 5% non-fat milk plus 0.1% Tween-20, blots were incubated with primary antibodies (1:500), horseradish peroxidase-conjugated secondary antibodies (1:3000; Amersham Pharmacia Biotech), and enhanced chemiluminescence reagents (Amersham Pharmacia Biotech).

To assay Chk2 activity, samples were lysed in 20 mM Tris-HCl (pH 8.0), 0.1% Triton X-100, 10 mM NaF, 1 mM NaVa₃VO₄, 10 mg/mL aprotinin, 1 mM PMSF, then incubated for 1 hr with antibody to Chk2 and protein A/G-Sepharose (Pharmacia). lmrunoprecipitates were washed and assayed in the presence of 30 μM CHKtide substrate peptide (KKKVSRSGLYRSPSMPENLNRPR (SEQ ID NO:24), Upstate Biotechnology, Inc), 40 μM adenosine triphosphate, 15 μCi [γ-³²P]ATP, for 30 min at 30° C. Proteins were resolved by electrophoresis in SDS-polyacrylamide gels and visualized by autoradiography. Aliquots of Chk2 immunoprecipitates were also used for Western blotting, allowing activity and content to be compared in the same samples.

### Example 9

### Telomere Attrition, Loss of TRF2, and Checkpoint Kinase Activation in Human Heart Failure

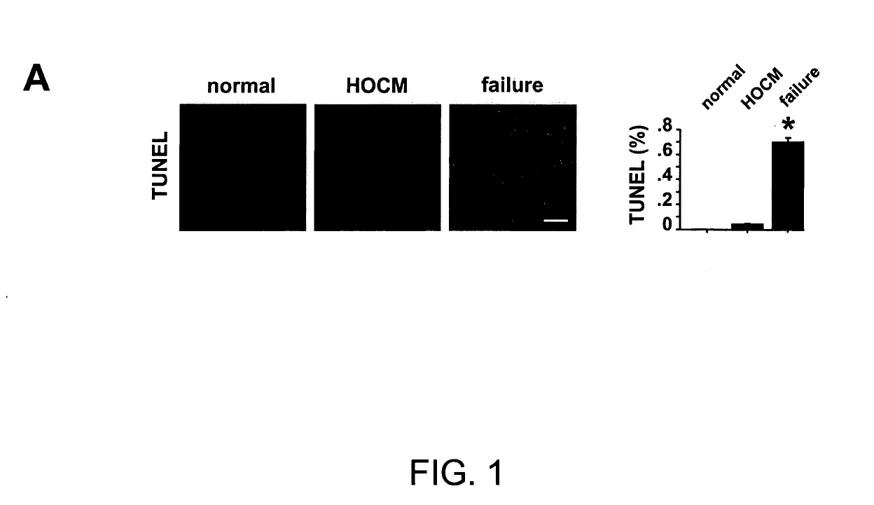

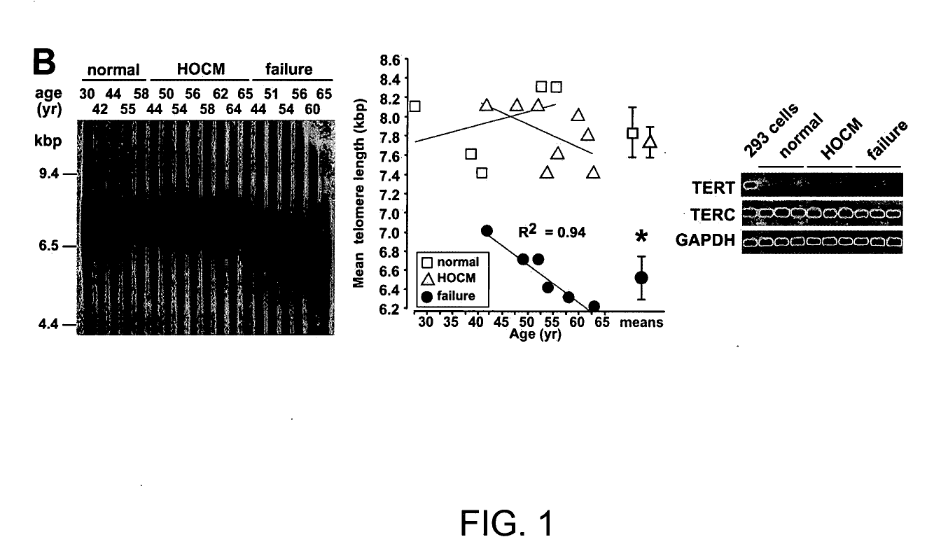

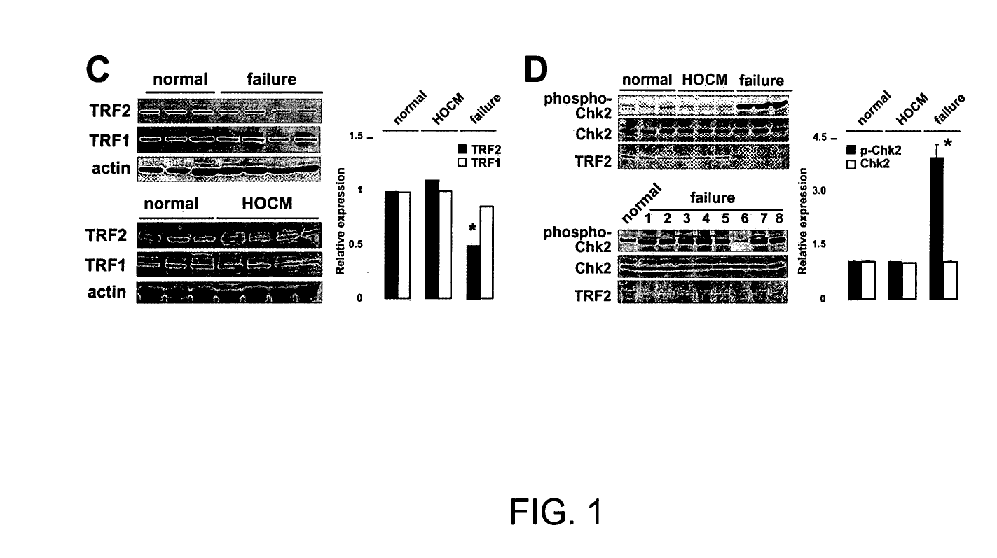

To address the expression and function of telomeric proteins in human heart disease, cardiac muscle from end-stage heart failure patients at the time of transplantation, HOCM undergoing therapeutic partial resection of the septum, and normal myocardium was analyzed. Samples were obtained as described in example 1. The prevalence of apoptosis (FIG. 1A) increased markedly in heart failure (0.70±0.04% by TUNEL assay; normal <0.005%, P=0.0001; HOCM 0.04±0.001%, P=0.0001; n=8 for each group), comparable to recent reports (Kang, P. M. & Izumo, S., 2000). TUNEL assays were conducted as described in example 5. Telomere length, as described in example 6, telomerase activity, as described in example 7, and TRF1/2 expression, as described in example 8, were determined using heart samples well matched for age and sex. Mean telomere length (FIG. 1B, left) was reduced 25% in failing hearts (6.5±0.2 kb), compared with normal samples (7.8±0.2 kb; P=0.0001) or HOCM patients (7.7±0.1 kb: P=0.0001). Although the RNA template for telomerase (TERC) was present in all three groups without significant difference, neither telomerase activity nor TERT expression was detected, in any of the three groups, using a telomeric repeat amplification protocol and RT-PCR for 30 cycles, respectively (FIG. 1B, right). The paucity of telomerase activity in adult human myocardium concurs with prior findings in mice (Oh, H. et al., 2001), and suggested a mechanism other than defective telomerase activity for the loss of telomere length in failing hearts.

To test one alternative mechanism for telomere dysftnction (Karlseder, J. et al, 1999; Multani, A. S. et al., 2000), TRF1 and TRF2 were examined (FIG. 1C). Both proteins were readily detected in normal adult human myocardium, with no change in HOCM. By contrast, in heart failure patients, TRF2 was down-regulated 50±8% (P=0.0001; range 25-75%). Interference with endogenous TRF2 activates apoptosis via the ataxia-telangiectasia mutated (ATM) protein kinase (Karlseder, J. et al., 1999), and partial loss of TRF2 was the earliest event in some forms of telomere shortening (Multani, A. S. et al., 2000). Consistent with this reported pathway, phosphorylation of Chk2 at Thr68, the principal site for activation by ATM (Melchionna, R. et al, 2000), was apparent in 12 of 14 failing hearts, but in none of the normal controls or HOCM patients (FIG. 1D). Chk2 levels were unaffected.

### Example 10

### Interference with Endogenous TRF2 Triggers Telomere Dysfunction and Apoptosis in Postmitotic Cardiomyocytes

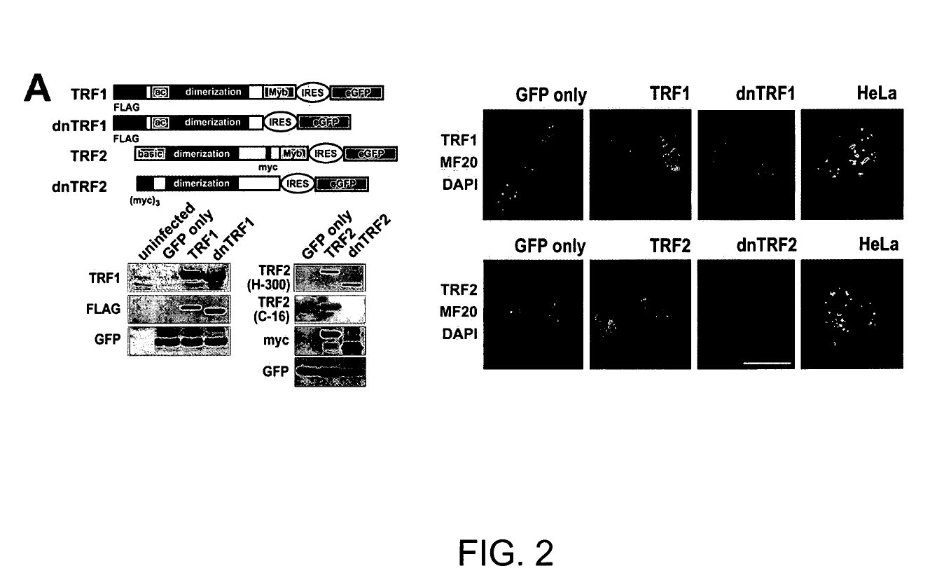

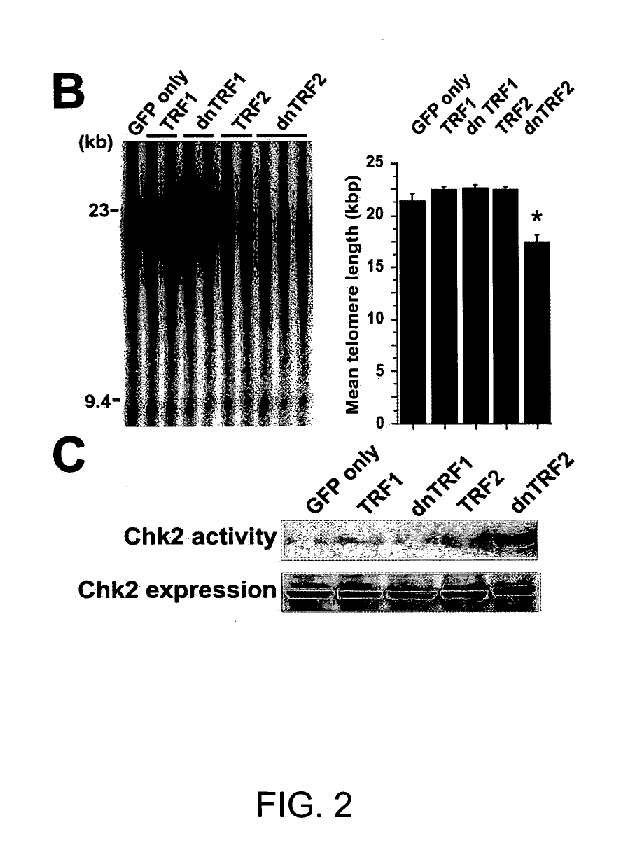

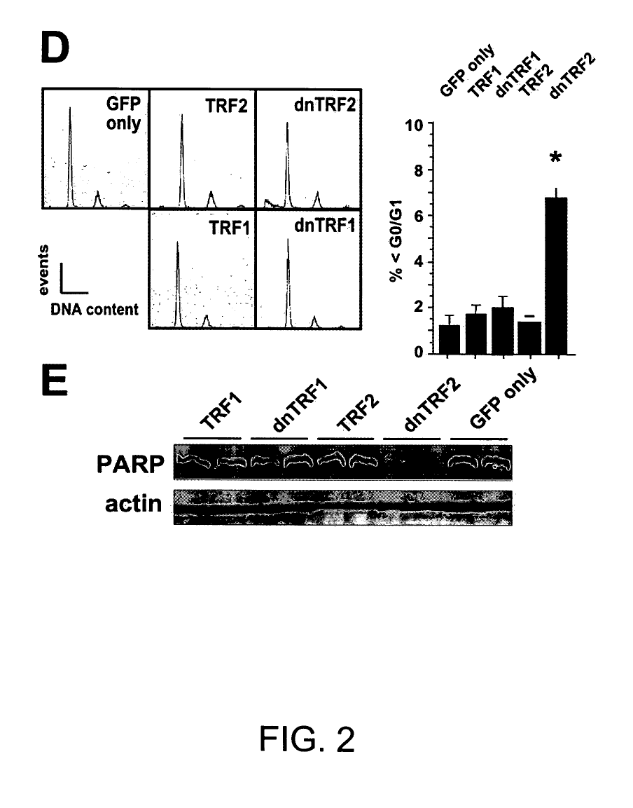

To ascertain if the inferred pathway from TRF2 to Chk2 was operative in post-mitotic cardiomyocytes (which might differ from cycling cells), epitope-tagged dominant-negative and wild-type TRF2 and TRF1 was expressed in primary culture using adenoviral vectors as described in example 2 (FIG. 2A). At the stage tested, cardiomyocytes were already growth-arrested in vivo and refractory to mitogenic serum (Oh, H. et al., 2001; Akli, S. et al., 1999). All four constructs were expressed uniformly. Staining was most intense in the nuclei, with a heterogeneous intranuclear distribution similar to that of endogenous TRF1/2 (FIG. 2A). Myc-tagged dominant-negative TRF2 induced telomere erosion (FIG. 2B), accompanied by Chk2 activation (FIG. 2C), PARP cleavage (indicative of caspase-3 activity, FIG. 2E), and apoptosis (FIG. 2D). Myc-tagged wild-type TRF2, FLAG tagged wild-type TRF1, and FLAG tagged dominant-negative TRF1 had no effect (FIG. 2B-2E).

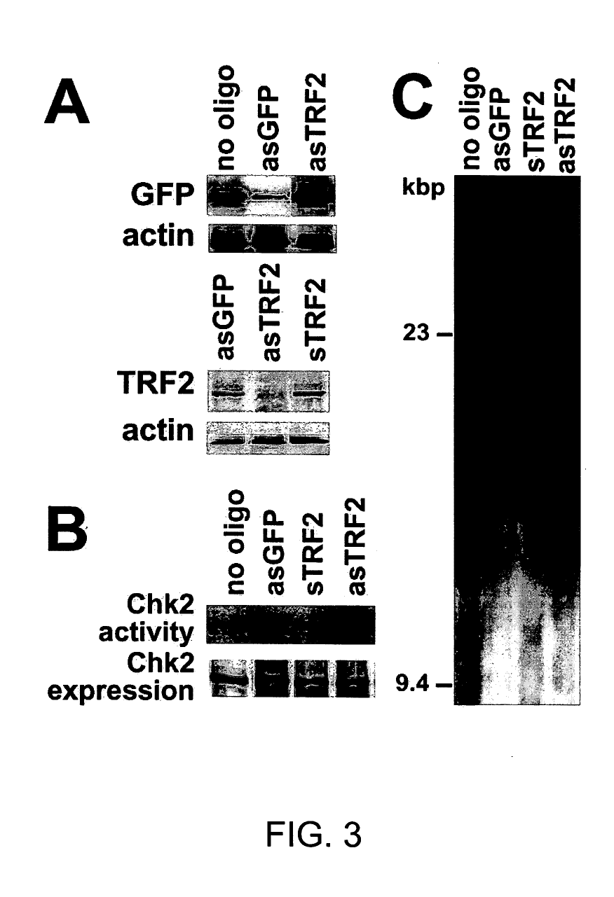

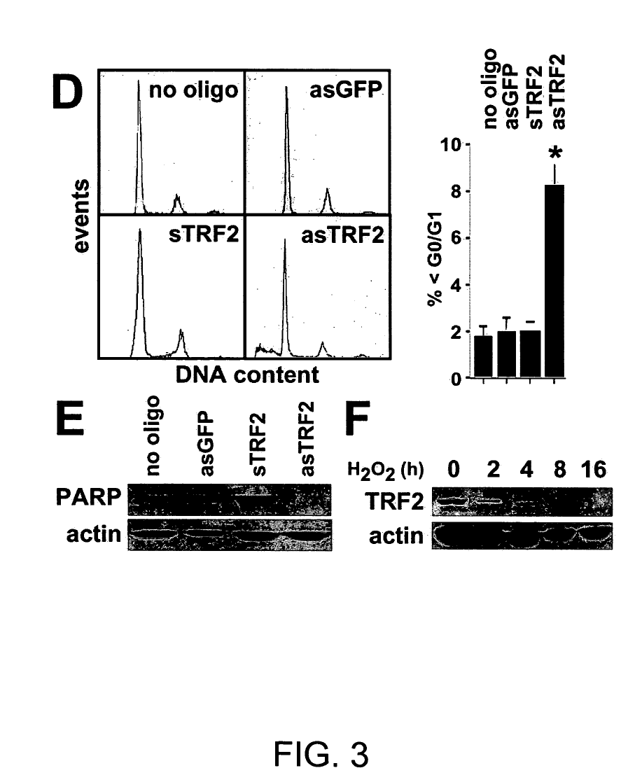

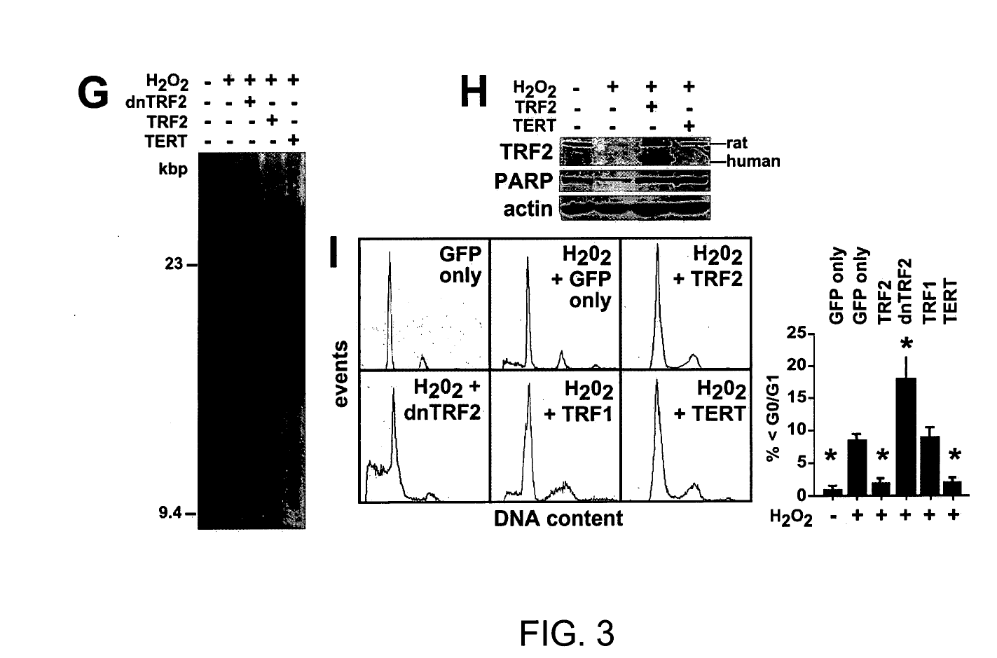

Because dominant-negative mutations are not formally equivalent to reduced expression, the above findings were confirmed using an antisense oligonucleotide for TRF2, versus the sense strand TRF2 control and an irrelevant antisense oligonucleotide against GFP as described in example 3. In cardiomyocytes, TRF2 and GFP were specifically reduced by the respective antisense oligos (FIG. 3A). Reduction of endogenous TRF2 provoked the same responses as did the dominant inhibitor: telomere shortening, Chk2 activation, PARP cleavage, and apoptosis (FIG. 3B-E). Thus, interference with TRF2 caused apoptosis and activation of Chk2 even in post-mitotic, non-cycling cells.

### Example 11

### TRF2 and TERT Protect Cardiomyocytes from Pathophysiological Stress

Endogenous TRF2 in cardiomyocytes decreased within 2 hr of oxidative stress (100 μM H202; FIG. 3F). Compared to a viral control expressing GFP alone, either TRF2 or TERT rescued the adverse effect of H₂O₂ on telomere length, PARP cleavage, and apoptosis (FIG. 3G-3D), consistent with earlier evidence for cardioprotection by TERT (Oh, H. et al., 2001). Dominant-negative TRF2 markedly potentiated the effect of H202 on apoptosis (FIG. 3I) but not on telomere length (FIG. 3G); thus, telomere attrition does not simply reflect the extent of apoptosis.

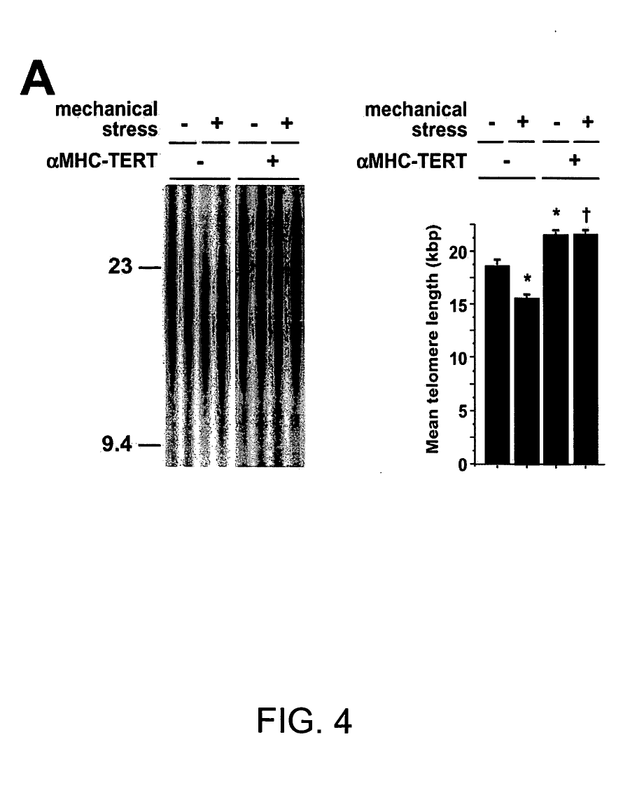

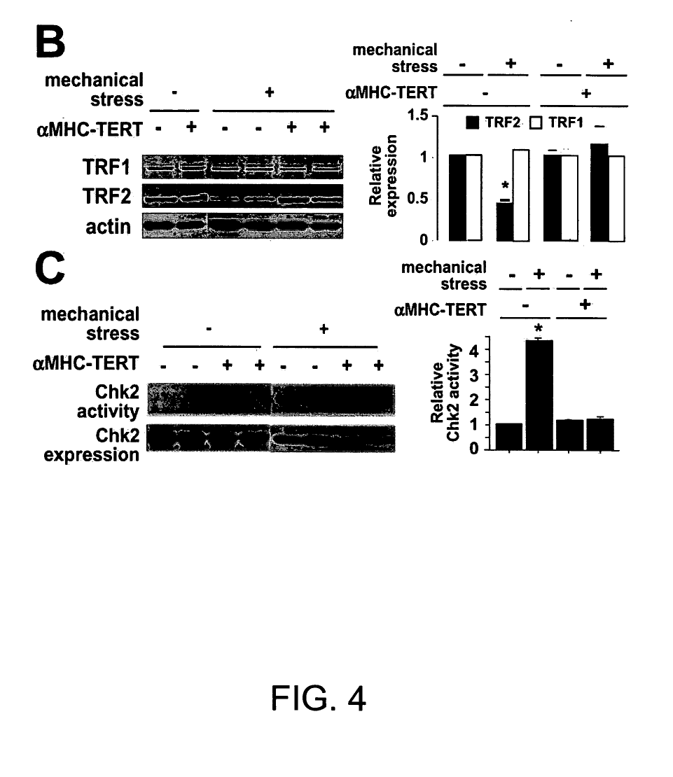

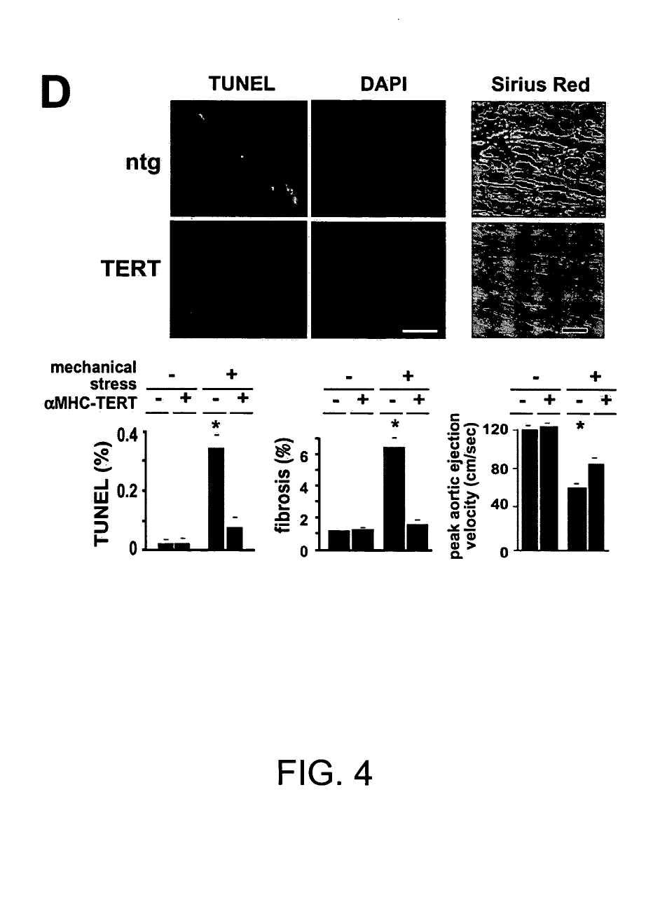

Mechanical load activated signaling cascades including oxidative stress (Frey, N. et al., 2003), predisposed cardiac muscle to late-onset apoptosis (Ding, B. et al., 2000), and can triggered apoptosis acutely, especially in susceptible backgrounds (Hirota, H. et al., 1999; Sadoshima, J. et al., 2002). To test if mechanical load induced telomere dysfunction in myocardium, adult mice were subjected to severe aortic constriction as described in example 4. By comparison to littermate controls undergoing the control procedure, telomere length was reduced 3 kbp by increased load for 7 d (n=4; P≦0.01; FIG. 4A). Under the conditions tested, mechanical load also triggered down-regulation of TRF2 by 52±2% (P≦0.001; FIG. 4B), induced Chk2 kinase activity (P=0.002; FIG. 4C), and induced apoptosis (0.32±0.06%; P=0.0003; FIG. 4D).

In culture, TERT largely prevented the loss of endogenous TRF2 provoked by oxidative stress (FIG. 3H). Forced expression of TERT in adult myocardium maintained telomere length and conferred protection from apoptosis after ischemia-reperfusion injury (Oh, H. et al, 2001). Next, it was tested to determine if TERT attenuated or rescued telomere dysfumction induced by severe mechanical load. As seen previously (Oh, H. et al., 2001), telomere length was 21.5±0.5 kbp in the αMHC-TERT mice, 3 kbp longer than wild-type littermates' (n=4; P≦0.01; FIG. 4A). By contrast to the sequelae of biomechanical stress in wild-type animals, αMHC-TERT mice were refractory to telomere erosion (FIG. 4A), loss of TRF2 (FIG. 4B), Chk2 kinase activation (FIG. 4C), and apoptosis (FIG. 4D). Consistent with the inhibition of cardiomyocyte death, αMHC-TERT mice had less replacement fibrosis after banding and better preservation of left ventricular ejection velocity, a measure of systolic function (FIG. 4D).

### Example 12

### HGK Transgenic Mice

As no adequate antibody to endogenous HGK exists, epitope-tagged HGK was expressed in mouse myocardium using the αMHC promoter (Subramaniam et al., 1991) and, also, using a conditional Cre/lox system (Gaussin et al., 2002). For the conditional system, FLAG-tagged wild-type HGK (Yao et al., 1999) was subdloned into the PstI-PstI fragment of pCAG-CATZ in lieu of LacZ, behind the loxP-flanked chloramphenicol acetyltransferase cassette providing the “stop” signal (Araki et al., 1995). The resulting plasmid, pCAG-CAT-HGK, was injected into the male pronucleus of fertilized FVB/N oocytes. Mice heterozygous for CAG-CAT-HGK were mated to ccMHC-Cre mice, to activate the transgene in cardiomyocytes (Gaussin et al., 2002). Experiments were performed in an isogenic FVB/N background. No early lethality resulted from cardiac expression of exogenous HGK, and αMHC-HGK was therefore used, except where noted, to simplify the breeding. αMHC-Gq mice and AMHC-TNFα mice were reported previously (Sakata et al., 1998; D'Angelo et al, 1997; Sivasubramanian et al., 2001).

Biomechanical stress was induced by partially occluding the transverse aorta in 6 week-old male mice (Sano et al., 2002). Only mice in which Doppler flow measurements confirmed severe occlusion (right-to-left carotid artery velocity ratio >3.5) were analyzed subsequently. The heart weight/body weight ratio, used to verify effective constriction, increased 20% at 7 days and 35% at 14 days. Ischemia/reperfusion was performed as described (Michael et al., 1995). For both surgical procedures, the control (“sham”) operation comprised anesthesia, thoracotomy, and placement of the ligature without occlusion.

### Example 13

### HGK Adenoviruses

To delineate the function of HGK, its effector TAK1, the TAK1 activator TAB1, and the terminal MAPK JNK1, recombinant adenoviruses were created expressing wild-type HGK, two catalytically inactive mutations (K54E, K54R), wild-type TAK1, dominant-negative TAK1 (K63W), TAB1, dominant-negative TAB1 (1-418) and dominant-negative JNK1 (APF). HGK was alternatively spliced, with the presence or absence of an SH3-like domain being one potentially important difference. Catalytically inactive, dominant-negative mutations of HGK (HGK K54E, HGK K54R) were generated by site-directed mutagenesis using wild-type human HGK cDNA, with the FLAG epitope, as template. Dominant-negative, FLAG-tagged JNK1 (JNK1 APF). Viruses were engineered using pAd-Easy-1 and pShuttleCMV. Adenoviruses encoding wild-type and dominant-negative TRF2 were constructed analogously (Oh et al., 2003), using cDNAs.

Ventricular myocytes from 1 to 2 day-old Sprague-Dawley rats were enzymatically dissociated, then subjected to Percoll gradient centrifugation and preplating to enrich for cardiomyocytes. After overnight culture in medium with 10% horse serum, cells were infected at a multiplicity of infection of 10, then cultured in serum-free medium for 24 to 48 hr (Oh et al., 2003). Where indicated, C2-ceramide (N-acetyl-D-sphingosine; ICN, Costa Mesa, Calif.), 5 mg/ml in dimethylsulfoxide (DMSO), was added at a final concentration of 20-50 μg/ml.

### Example 14

### HGK Immunocytochemistry

Cells were fixed with 10% neutral buffered formalin and permeabilized with 0.2% Triton X-100 in phosphate-buffered saline. Recombinant HGK, TAB1 and JNK1 were labeled using 10 μg/ml mouse M2 anti-FLAG antibody (Sigma, St. Louis, Mo.) and 2 μg/ml FITC-conjugated goat antibody to mouse IgG (Molecular Probes, Eugene, Oreg. 97402). Recombinant TAK1s were labeled using 10 μg/ml mouse monoclonal anti-HA (12CA5) antibody (Roche Applied Science, Indianapolis, Ind.). Myocyte identity was confirmed using 10 μg/ml mouse antibody to sarcomeric tropomyosin (T9283; Sigma) conjugated directly with Texas Red-X succinimidyl ester (F-6162; Molecular Probes, Eugene, Oreg.). Nuclei were stained with 2.5 μg/ml diamidinophenolindole (DAPI). Images were captured with a Zeiss Axioplan 2 epifluorescence microscope.

### Example 15

### HGK Western Blotting and Immune Complex Kinase Assays

Cells were lysed in 20 mM HEPES, pH 7.4, 2 mM EGTA, 50 mM glycerophosphate, 1% Triton X-100, 10% glycerol, 1 mM dithiothreitol, 2 μg/ml leupeptin, 5 μg/ml aprotinin, 1 mM phenylmethylsulfonyl fluoride, 1 mM Na₃VO₄. Lysates were resolved by SDS-polyacrylamide gel electrophoresis and transferred to optitran (Schleicher & Schuell, Keene, N.H.) membranes for Western blotting. Rabbit antibodies to ERK, phospho-ERK (Thr202/Tyr204), JNK, phospho-JNY (Thr183/Tyr185), p38 and phospho-p38 (Thr180/Tyr182) were purchased from Cell Signaling (Beverly, Mass.). Mouse monoclonal antibody against human Bcl-2, rabbit antibody to PARP, and goat antibody to total actin were from Santa Cruz Biotechnology (Santa Cruz, Calif.). Protein expression was visualized using horseradish peroxidase-conjugated second antibodies and enhanced chemiluminescence reagents from Amersham Pharmacia Biotech (Piscataway, N.J.).

For HGK immune complex kinase assays, recombinant HGK was precipitated using M2 antibody and protein G-Sepharose, in the lysis buffer above. Precipitates were washed twice in lysis buffer, twice with 500 mM LiCl, 100 mM Tris-HCl, pH 7.6, 0.1% Triton X-100, and twice with kinase buffer (20 mM MOPS, pH 7.6, 2 mM EGTA, 10 mM MgCl₂, 1 mM dithiothreitol, 0.1% Triton X-100, 1 mM Na₃VO₄), then were mixed with 10 μg of myelin basic protein (MBP) (Invitrogen, Carlsbad, Calif.), as substrate, 15 μM ATP, and 10 μCi [γ-³²P]ATP in 30 μl of kinase buffer for 30 min at 30° C. (Yao et al., 1999). Reaction mixtures were resolved by SDS-polyacrylamide gel electrophoresis, then were analysed by autoradiography and Western blotting as above.

### Example 16

### HGK Activates the Mitochondrial Death Pathway

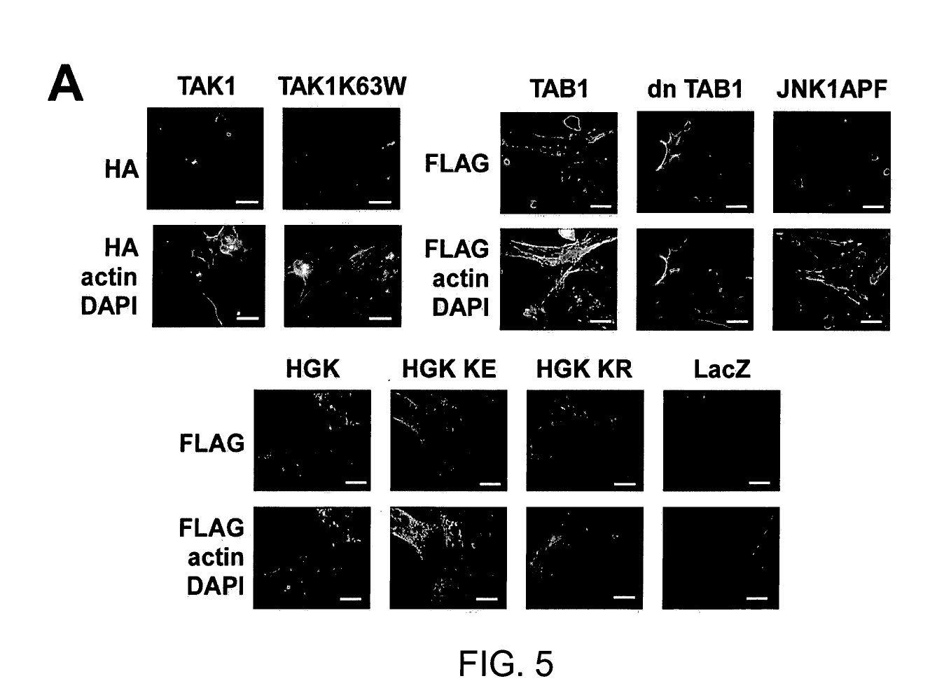

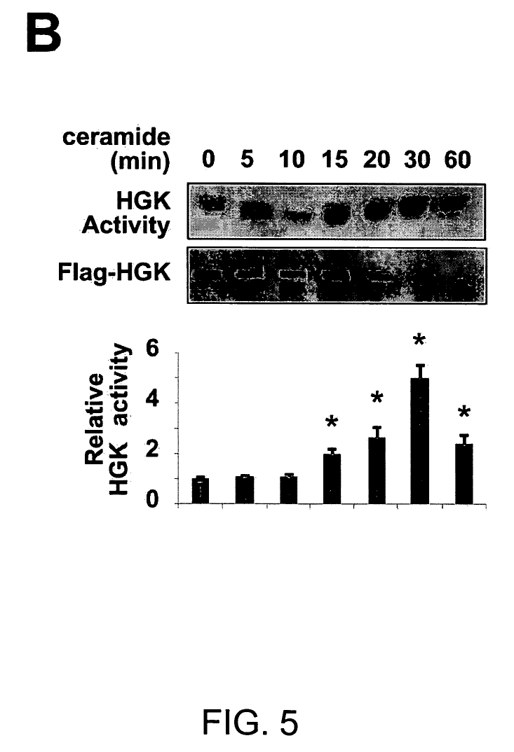

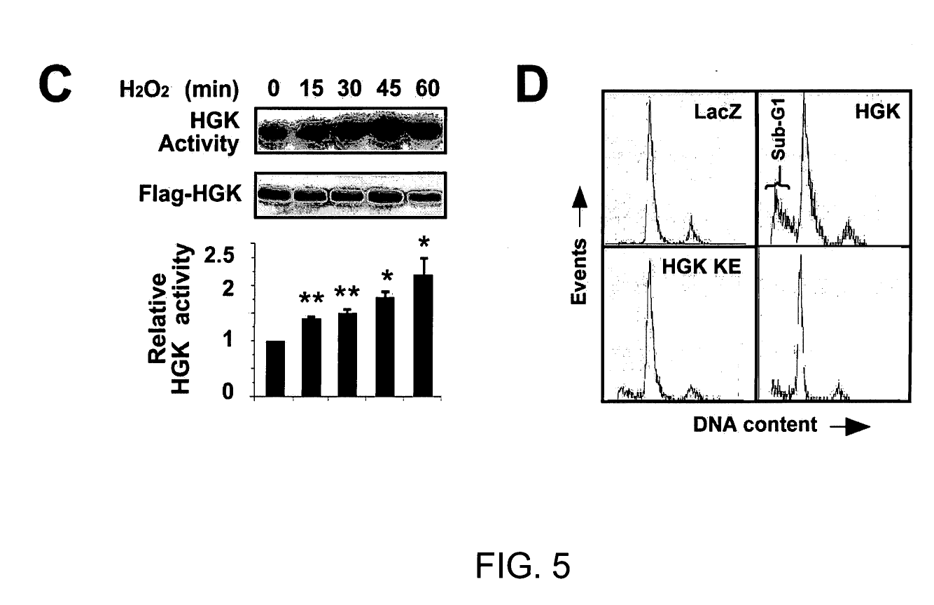

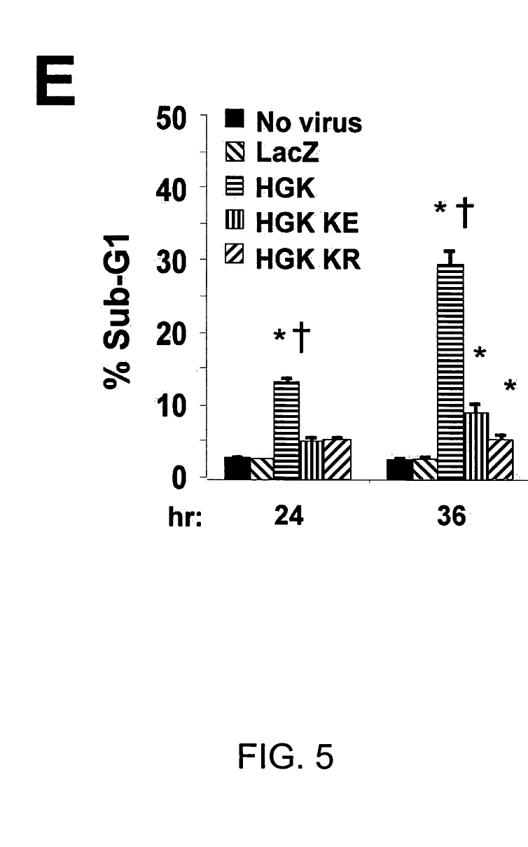

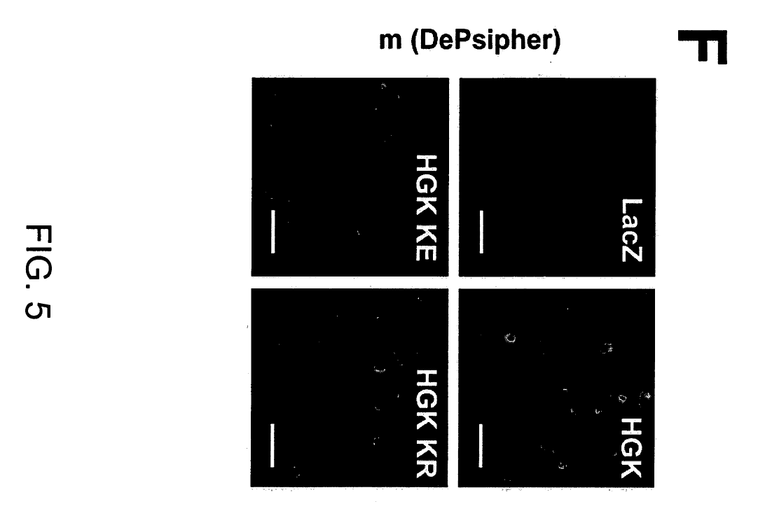

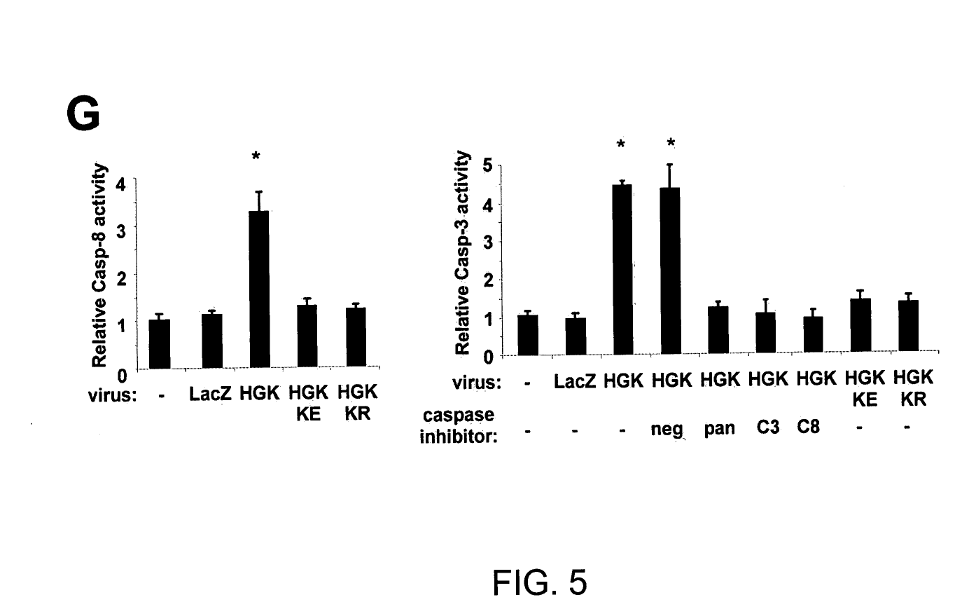

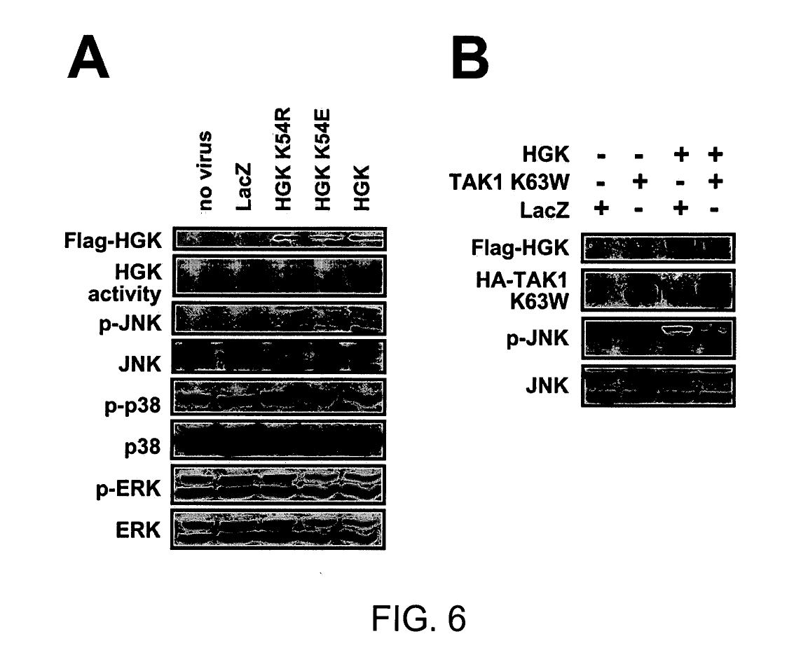

Adenoviruses for HGK, TAK1, TAB1 and Gq were used singly and in combination, with virus encoding LacZ to control for multiplicity of infection. For all viruses, the efficiency of infection was >95% (FIG. SA). Epitope-tagged HGK was catalytically active after viral delivery and activated further by ceramide, a mediator of relevant apoptotic pathways in cardiac muscle including ischemia/reperfusion, oxidative stress, and TNFα (FIG. 5B) (Levade et al., 2001; Suematsu et al., 2003). HGK was also activated by oxidative stress itself (H₂O₂; FIG. 5C). Under these conditions, exogenous wild-type HGK provoked measurable autoactivation even in the absence of agonist (FIG. 5B), as reported in other backgrounds (Yao et al., 1999).

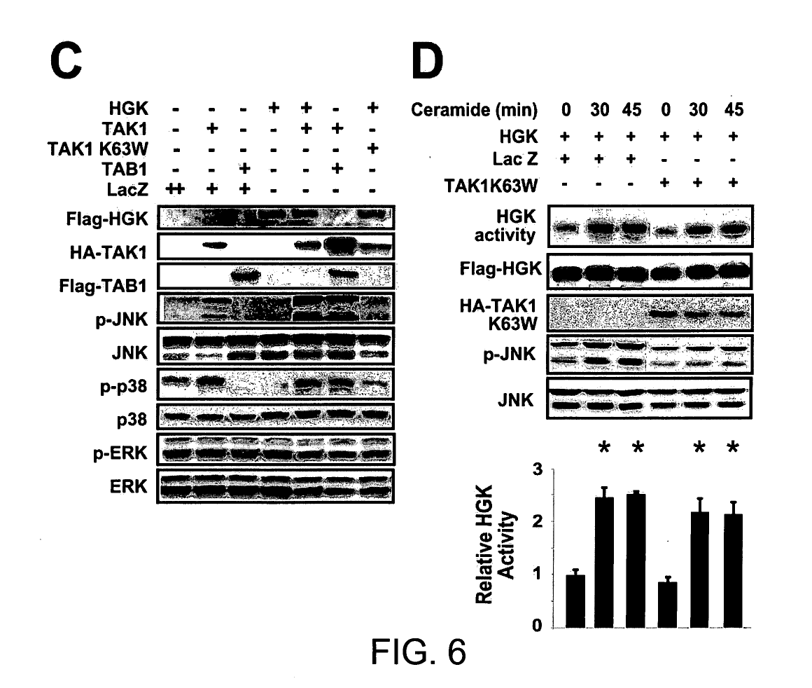

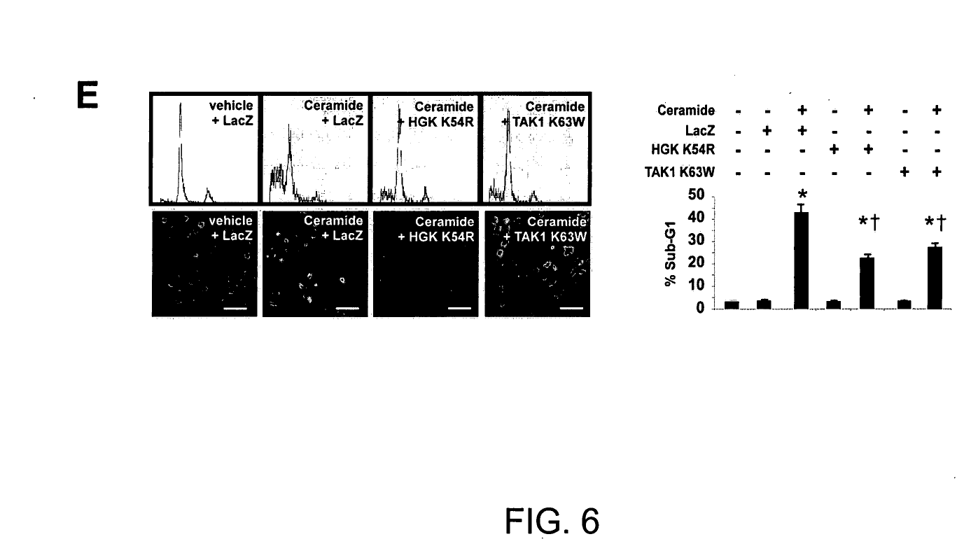

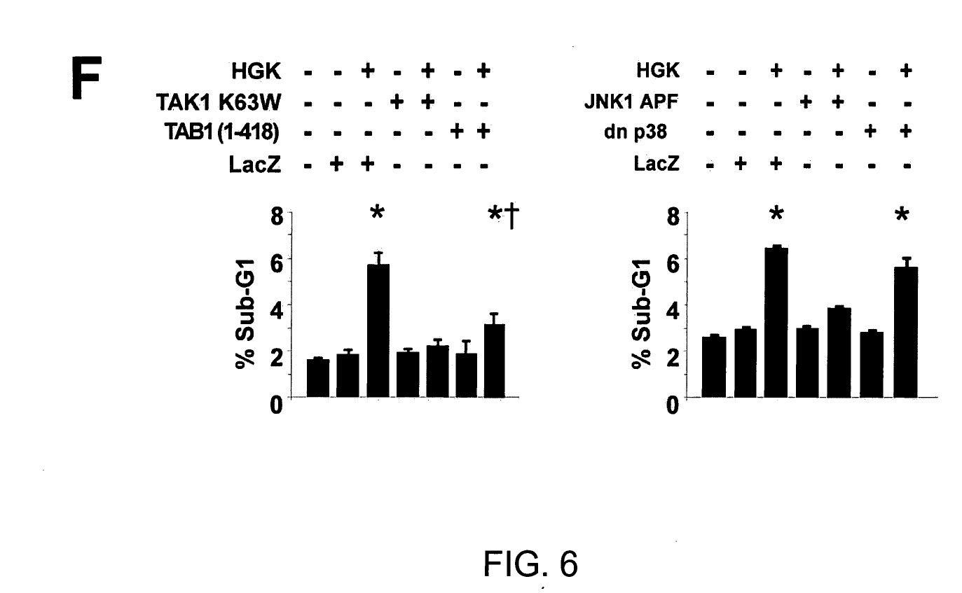

Next, to test if signaling was contingent on the activity of HGK, cells were subjected to virus encoding LacZ, wild-type HGK, or catalytically inactive HGK (K54E and K54R). All three forms were expressed at equivalent prevalence, cytoplasmic localization, and abundance (FIGS. 5A, 6A). As expected, kinase activity was detected exclusively with wild-type HGK (FIG. 6A). Apoptosis was assessed by two-color flow cytometry for hypodiploid cardiomyocytes. Exogenous HGK increased the proportion of apoptotic cells 4-fold, compared to virus encoding LacZ (FIGS. 5D, 5E). Despite the lack of kinase activity, the catalytically inactive mutations HGK (K54E and K54R) triggered apoptosis at 36 hr (FIGS. 5D, 5E) and later time-points. This result concured with known properties of GCK-like kinases including NIK, which activated the JNK pathway even as a kinase-dead mutation, via its C-terminal citron homology domain (Su et al., 1997) (see FIG. 6A).

Dissipation of mitochondrial potential, ΔΨm, was measured by the fluorescence of 5, 5′, 6, 6′-tetrachloro-1, 1′, 3, 3′-tetraethylbenzimidazolyl carbocyanine iodide (DePsipher; FIGS. 5F, 6E). When ΔΨm was intact, mitochondrial uptake and aggregation of the dye resulted in fluorescence; when ΔΨm was disturbed, the dye diffused to the cytoplasm and reverted to its monomeric form. In control cells, fluorescence predominated; diffuse fluorescence was common in HGK-treated cells, indicating dissipation of ΔΨm; and an intermediate phenotype was seen with catalytically inactive HGK. HGK induced more than 4-fold the activity of caspase-3, the “executioner” caspase downstream of the mitochondrial death pathway. HGK also activated caspase-8 (FIG. 5G), as expected from the reported role of HGK as a proximal effector of “death domain” receptors (Yao et al., 1997).

### Example 17

### HGK-Induced Apoptosis Requires the TAK1-JNK Death Pathway

As measured using activation-specific phospho-epitopes, JNK the terminal MAPK most affected by HGK (FIGS. 6A-6C), and was activated, much more weakly, even by the kinase-inactive mutations (FIG. 6A; see (Sue et al., 1997)).

To test if TAK1 was essential for HGK signal transduction, HGK was co-infected into the cells with kinase-deficient, dnTAK1 (K63W). The activation of JNK caused by HGK was blocked almost completely by dnTAK1 (FIGS. 6B, 6C). By contrast, dnTAK1 had no significant effect on ceramide-induced HGK activity (FIG. 6D).

Next, to test if kinase-inactive mutations of HGK and TAK1 promoted the survival of cardiomyocytes challenged with ceramide, cells infected as above were analyzed for apoptosis, using ΔΨm and the hypodiploid fraction. HGK K54R and TAK1 K63W markedly impaired the dissipation of ΔΨm by ceramide (FIG. 6E, left). Ceramide induced a 20-fold increase in hypodiploid myocytes, attentuated ˜50% by kinase-deficient TAK1 (K63W) and HGK (K54R) (FIG. 6E, right). Differences between these assays in the magnitude of protection observed reflected technical issues, or residual levels of signal through the “mitochondrial” versus “death receptor” apoptosis pathways (Aza-Blanc et al., 2003).

Next, cardiomyocytes were subjected to gene transfer with wild-type HGK in the absence or presence of dominant-interfering mutations of TAK1 (K63W), the TAK1 activator TAB1 (1-418), JNK1 (APF), and p38α (AGF) (FIG. 6F). HGK-induced apoptosis was blocked almost completely by TAK1 K63W. Less complete inhibition was seen with dominant-negative TAB1. JNK1 APF suppressed HGK-induced apoptosis >80%; p38α AGF conferred no significant protection (FIG. 6F). These results suggested that TAK1 and JNK1 were the predominant effectors for HGK-induced apoptosis, whereas ceramide likely utilized other effectors besides just the HGK-TAK1 module.

### Example 18

### HGK Activity is Coupled, Reciprocally, to Levels of the Telomere-Capping Protein TRF2

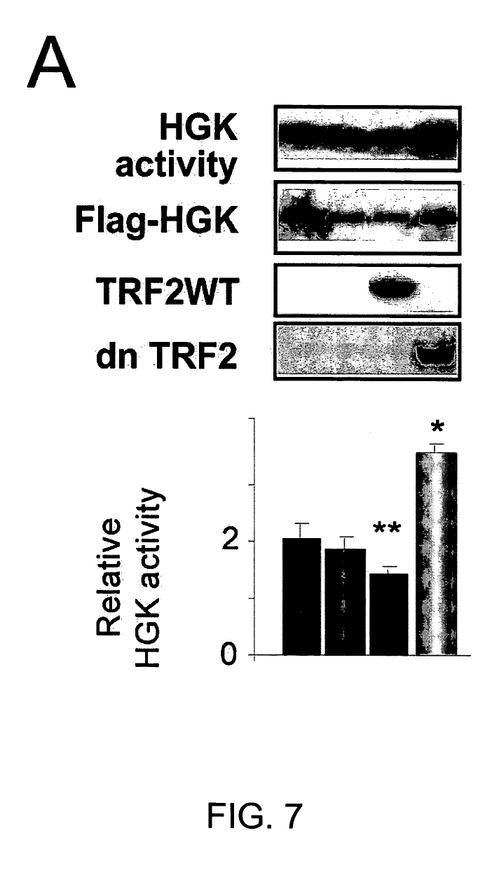

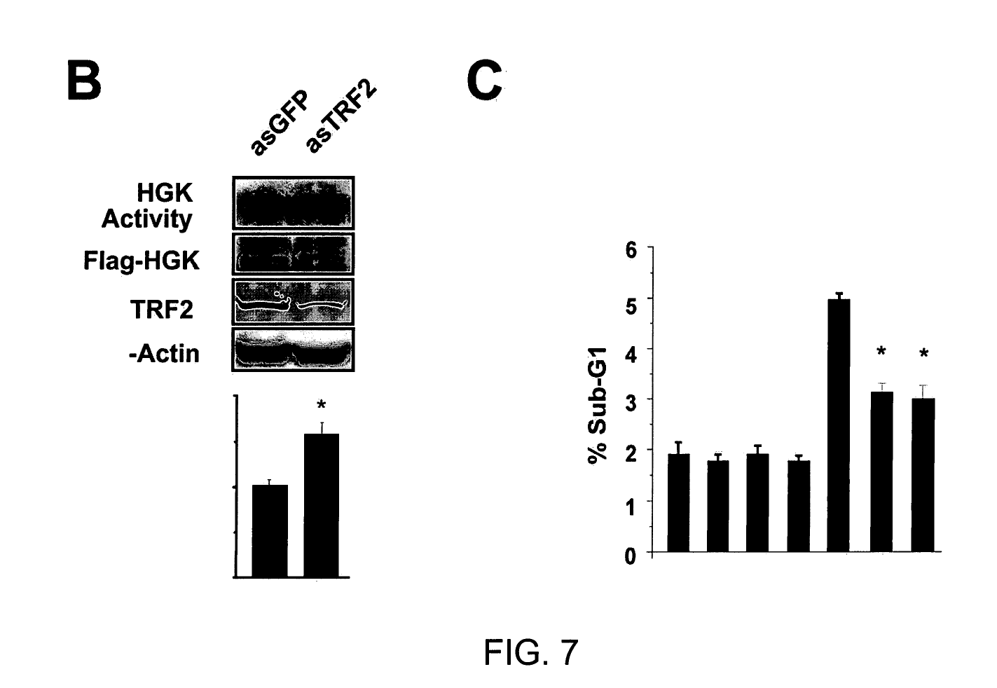

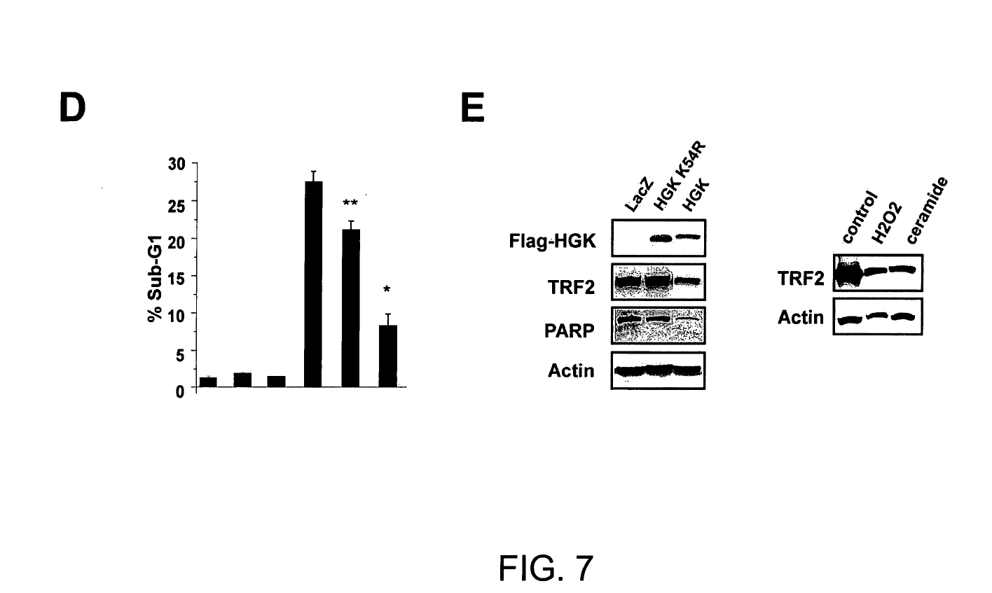

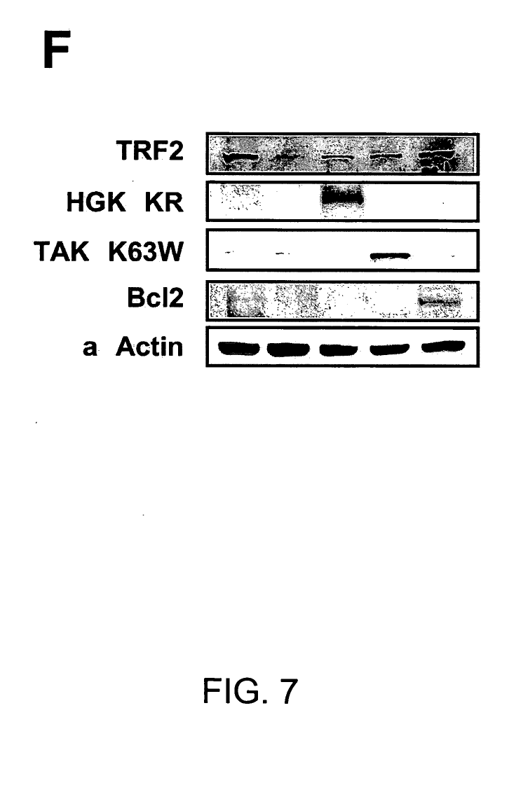

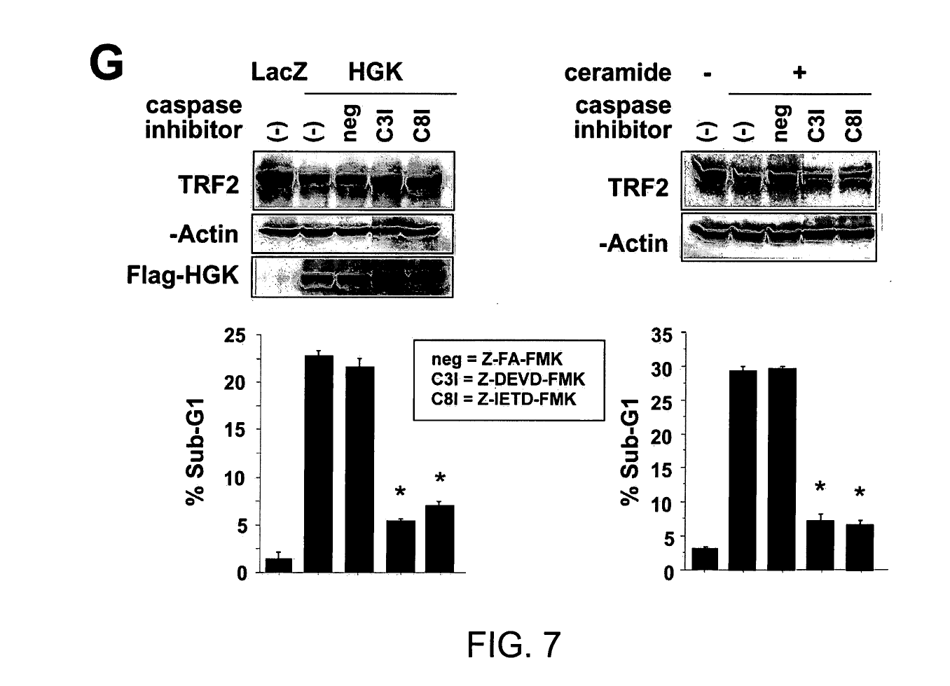

Inhibition of TRF2 function or expression in cardiomyocytes suffices to incite telomere shortening, Chk2 activation, and apoptosis (Oh et al., 2003). In addition, DNA damage can induce ceramide accumulation (Liao et al., 1999), and ceramide was a potent activator of HGK. HGK activity was induced 2-3 fold by dominant-negative TRF2 (FIG. 7A). Conversely, basal HGK activity was decreased 35% by wild-type TRF2 (FIG. 7A). As an independent test of this connection, endogenous TRF2 expression was suppressed with antisense oligonucleotides, using antisense reduction of GFP as an irrelevant control (Oh et al., 2003). Based upon suppression of TRF2 overexpression, HGK activity was induced more than 50% by reducing TRF2 (FIG. 7B).

To test the prediction, based on this finding, that interference with telomere function caused apoptosis via the TAK1-JNK1 pathway, cardiomyocytes were infected with dominant-negative TRF2, with or without dnTAK1 and dnJNK1. Apoptosis induced by dominant-negative TRF2 was blocked 80% by either (FIG. 7C). Conversely, HGK-induced apoptosis was partially blocked by TRF2, which concurs with the dampening of HGK activity by TRF2. This effect was partial, by contrast to the more complete block by Bcl-2, which acted directly on mitochondrial permeability (FIG. 7D).

Hence, TRF2 was down-regulated in culture by each of the signals that activated HGK (oxidative stress, ceramide), similar to what was found in vivo with pressure-overload (Oh et al., 2003) and ischemia/reperfusion.

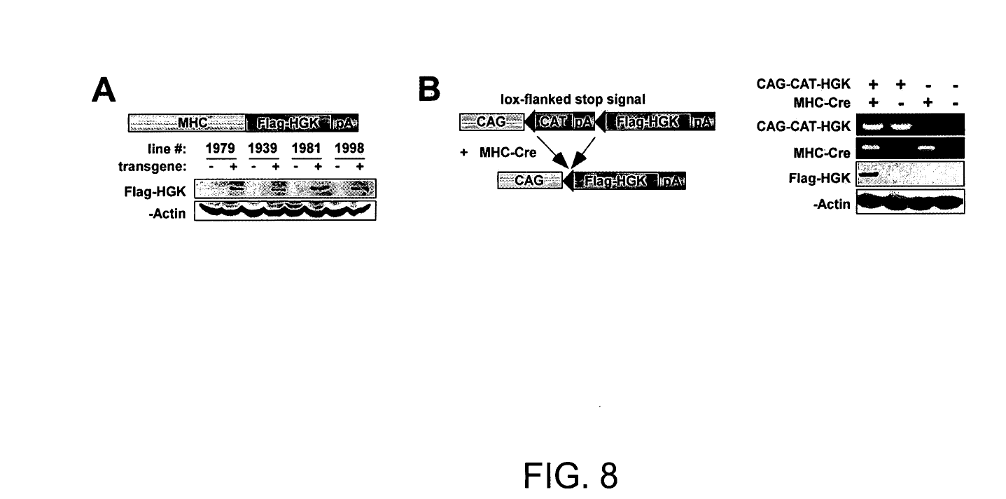

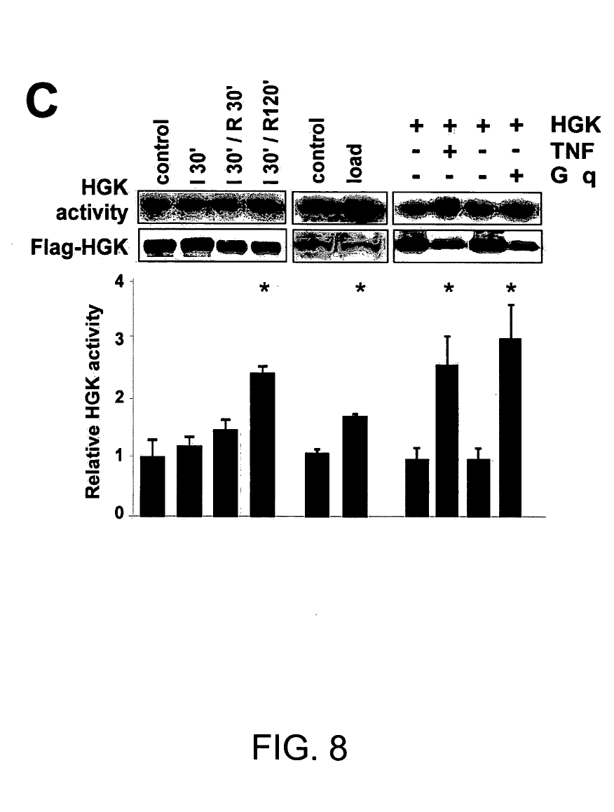

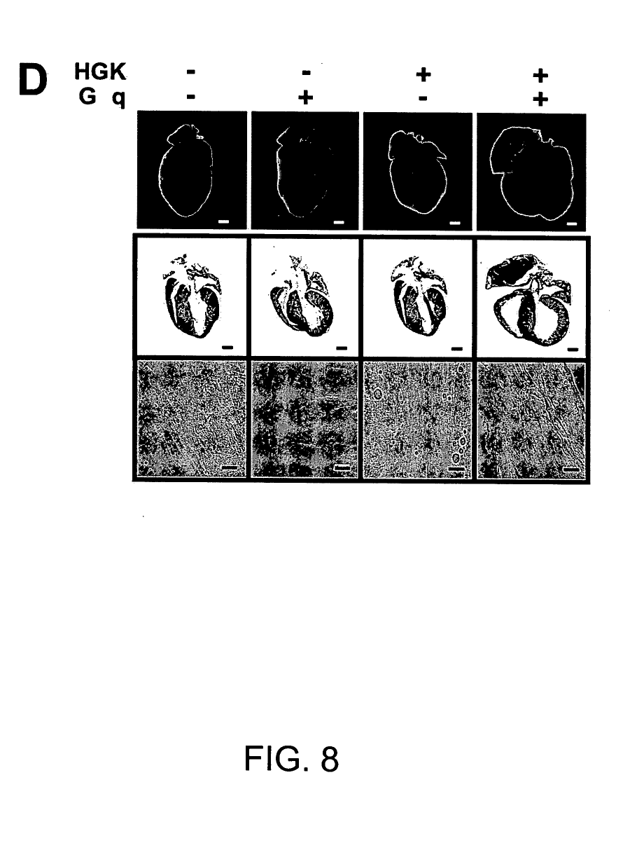

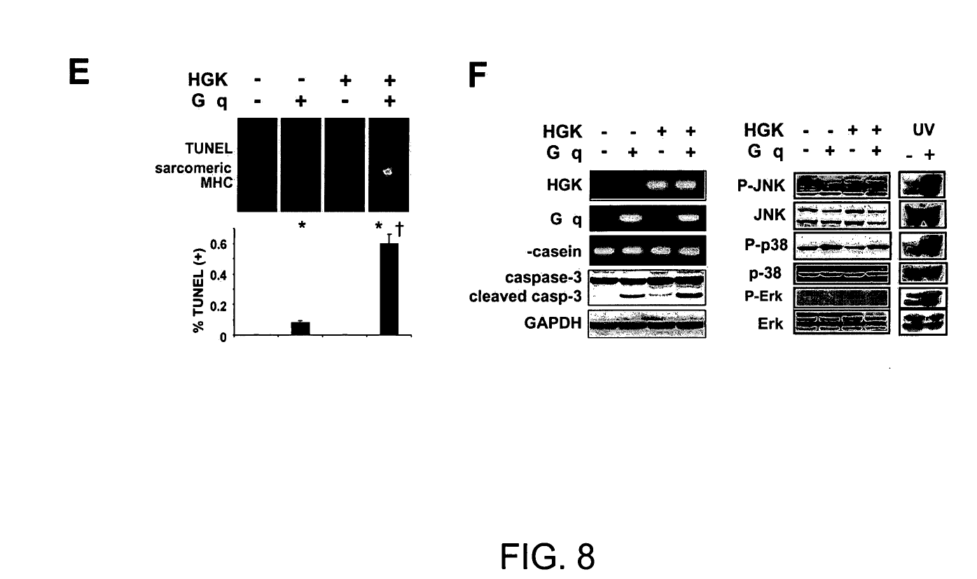

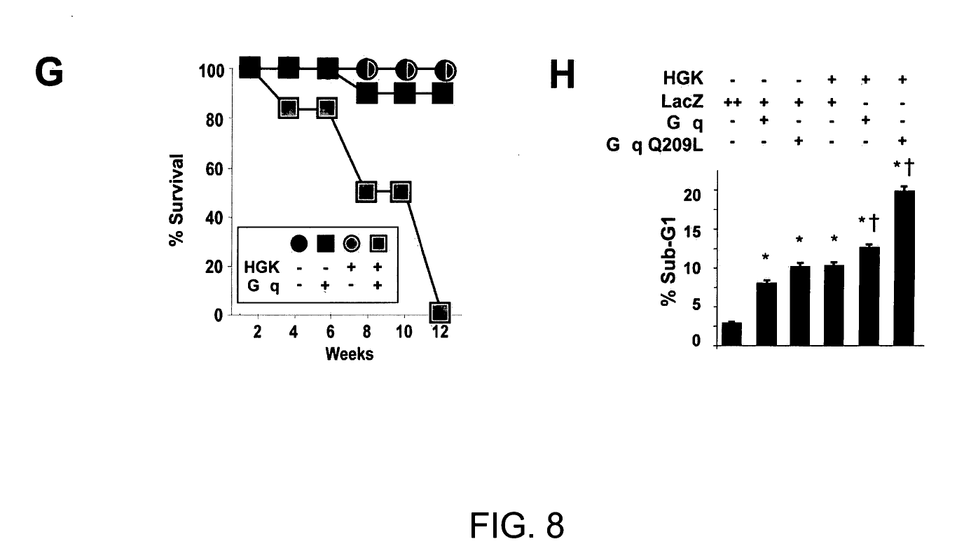

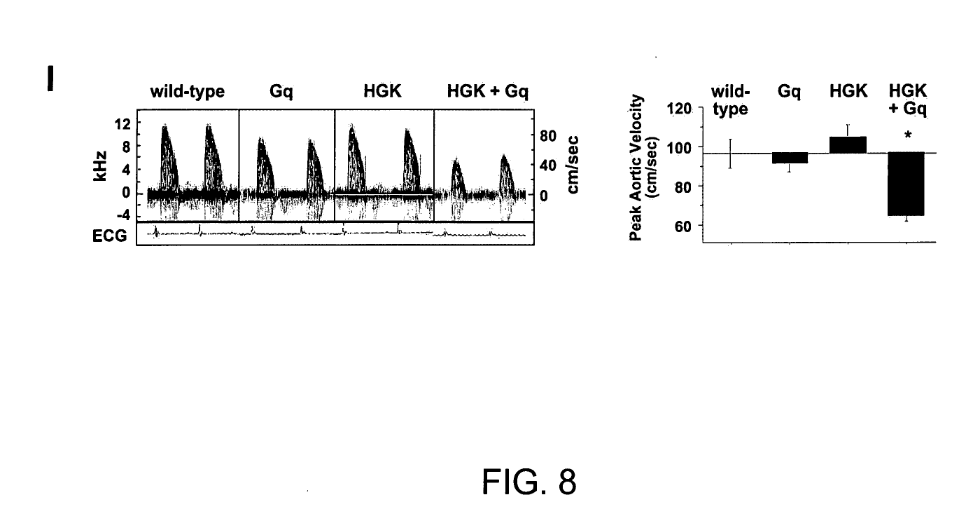

Next, HGK activity was increased to determine if an increase in HGK activity inhibited TRF2 levels. Indeed, TRF2 was down-regulated, accompanied by PARP cleavage, by viral delivery of HGK, but not HGK K54R (FIG. 7E). Ceramide reduced TRF2 levels (FIG. 7E), as was shown for oxidative stress (Oh et al., 2003). Ceramide-induced TRF2 down-regulation was blocked partially by TAK1 K63W or JNK1 APF, and nearly completely by Bcl-2 (FIG. 7F). Down-regulation of TRF2 by HGK was caspase-dependent (FIG. 8G, upper left), whereas down-regulation of TRF2 by ceramide was refractory to caspase inhibitors (FIG. 8G, upper right). By contrast, apoptosis induced by ceramide or by HGK, was successfully blocked by the caspase-3 and caspase-8 inhibitors (FIG. 8G, lower panels). Together, these results signified that loss of TRF2 protein was not just a consequence of apoptosis, and that mediators other than caspases down-regulate TRF2, in some settings.

### Example 19

### HGK is Activated by and Potentiates Cardiac Death Signals

To facilitate analyzing HGK activity in myocardium, transgenic mice were created for conventional and Cre-dependent cardiac-specific expression of epitope-tagged HGK (FIGS. 8A, 8B). Both systems were cardiac-restricted. In the latter case, epitope-tagged HGK was detected only in myocardium of animals co-inheriting both the latent transgene (CAG-CAT-HGK) and cardiomyocyte-specific Cre (αMHC-Cre). Four independent aMHC-HGK founder lines were generated (FIG. 8A), with no obvious baseline phenotype.

HGK-expressing mice were subjected to four complementary provocations of cardiac apoptosis (FIG. 8C): ischemia/reperfusion injury, mechanical load, and transgenic expression of TNFα or Gq. All four induced HGK activity: Ischemia/reperfusion 2-fold; mechanical load by 45% at 7 d and 60% at 14 d; αMHC-TNFα 2-fold; αMHC-Gq 2-fold (N=4, P<0.001, for all comparisons). HGK/Gq double transgenic mice were chosen for longer-term follow-up, as adverse synergies were known for Gq with other cardiac stress pathways (Yussman et al., 2002; and Sakata et al., 1998). The 25-copy αMHC-Gq line was used. This transgenic mouse line was well tolerated on its own, but conferred a predisposition to apoptosis. By the age of 10 weeks, HGK/Gq bigenic mice developed cardiac enlargement. Although the increase in mass was no greater than with Gq singly, the combined effect of Gq plus HGK was ventricular dilatation with apoptosis evidenced by TUNEL staining and the cleaved, activated form of caspase 3 (FIG. 8D-F). HGK/Gq mouse myocardium also showed enhanced JNK activation (FIG. 8F).

To obviate secondary hemodynamic or systemic effects as the basis for apoptosis, fuctional interaction between HGK and Gq was studied by viral gene transfer to cultured cardiomyocytes. Over-expressing wild-type Gq increased apoptosis 3-fold, activated Gq increased apoptosis 4-fold, and the effects of HGK plus Gq were roughly additive in these short-term studies. (FIG. 8H)

All mice co-inheriting both transgenes died by 3 months of age with dilated cardiomyopathy (FIG. 8G) and severely diminished systolic function (FIG. 8I). No apoptosis, dysfumction, or mortality resulted from HGK alone.

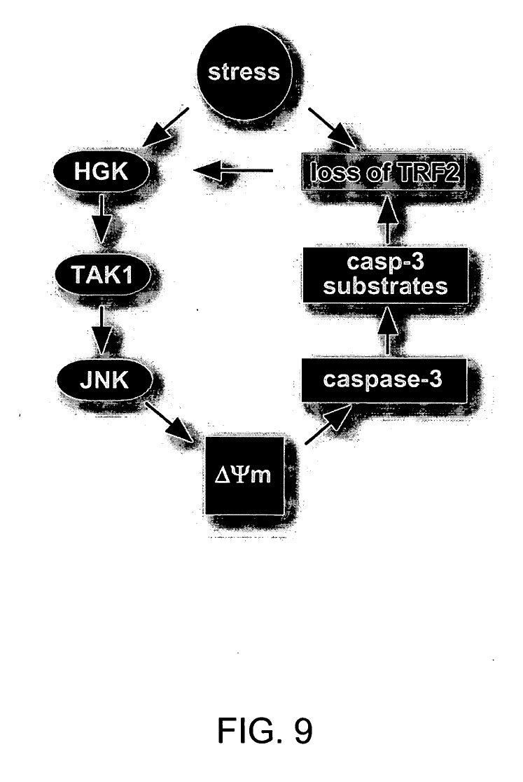

Thus, the HGK-TAK1 pathway is coupled, reciprocally, to telomere dysfimction from loss of TRF2, a novel feed-forward cycle for apoptotic signals (FIG. 9). It is further envisioned that caspase-8 activates caspase-3 both directly and via the “mitochondrial” pathway, by cleavage of Bid.

### Example 20

### TRF2 Animal Models

Cardiac-specific transgenic mice were created by subcloning the TRF2 and dnTRF2 cDNAs behind the 5.5 kb mouse αMHC promoter (Subramaniam et al., 1991). The resultant plasmids were injected into the pronuclei of fertilized FVB/N oocytes, and tail DNA was used to screen for inheritance of the transgenes. Doppler and M-mode echocardiography were performed as described (Oh et al., 2003; and Minamino et al., 2002).

### Example 21

### Histology

Hearts were pressure-perfused with formalin, dehydrated to 70% ethanol, mounted in paraffin, sectioned, and stained with hematoxylin and eosin or Gomori-Trichrome. To confirm appropriate nuclear expression of the transgenes, immunohistochemistry was performed. Slides were de-paraffinized, dehydrated, washed with PBS, and treated with 0.4% Triton-X in PBS. Slides were then incubated sequentially with mouse antibody to sacromeric α-actin (Sigma) and Texas Red-conjugated antibody to mouse IgG for labeling cardiomyocytes, then with rabbit anti-TRF2 and FITC-conjugated antibody to rabbit IgG. Nuclei were stained with 4′,6-diamidino-2-phenylindole (DAPI). Images were captured with a Zeiss Axioplan 2 epifluorescence microscope.

### Example 22

### Doxorubicin Down-Regulates TRF2 and Activates the DNA Damage Pathway

Anthracycline chemotherapeutic agents induce an irreversible, cumulative cardiomyopathy, with existing clinical interventions ineffective (Keefe et al., 2001) and apoptosis as a likely underlying mechanism (Zhu et al., 1999; and Dowd et al., 2001).

To test for potential operation of a TRF2-dependent death pathway, TRF2 levels were measured in cardiac myocytes treated with 1 μM doxorubicin. TRF2 protein expression was decreased by 50% within 8 hr. By contrast, the loss of poly-(ADP ribose) polymerase (PARP), indicative of caspase-3 activation in apoptosis, was not comparable until 48 hr had elapsed. Thus, down-regulation of TRF2 was an early response to doxorubicin, compared to a canonical caspase-3 substrate.

Using flow cytometry to detect hypodiploid cardiac myocytes, doxorubicin was administered to myocytes to determine that doxorubicin induced cardiac myocyte apoptosis. Importantly, adenovirus encoding TRF2 plus GFP reduced myocyte apoptosis by 75%, compared to a control virus encoding GFP alone (3.1±1.0% versus 12.2±0.5%; n=6; P<0.0001). Thus, the loss of endogenous TRF2 provoked by doxorubicin and the rescue by exogenous TRF2 suggested that doxorubicin caused apoptosis in myocytes in part by perturbing normal TRF2 protein abundance. These paired conclusions paralleled the above findings that oxidative stress caused the loss of endogenous TRF2 and induced apoptosis in cultured cardiac myocytes, whereas exogenous TRF2 protected the cells.

Although the checkpoint kinase ATM is best known in connection with cells' response to double-strand DNA breaks (Bakkenist et al., 2003), telomere dysfunction resulting from the loss of TRF2 fimction also activates this pathway (Oh et al., 2003; Karlseder et al., 2002; and Takai et al., 2003). To test if TRF2 was sufficient to inhibit activation of the ATM-dependent DNA damage pathway by doxorubicin (Panta et al., 2004), the phosphorylation of H2AX was measured at serine 139 and p53 at serine 15, two specific sites of action for ATM (Shiloh 2003). Significantly, adenovirus-mediated expression of TRF2 blunted the phosphorylation of both H2AX and p53. Western blotting for total PARP also demonstrated that TRF2 protected myocytes from doxorubicin-induced apoptosis.

### Example 23

### Cardiac-Specific TRF2 Mice are Resistant to Doxorubicin Cardiomyopathy

To test for an equivalent protective role of TRF2 against doxorubicin-induced myocyte apoptosis in the intact heart, Myc-tagged TRF2 was expressed selectively in mouse myocardium using the αMHC promoter. Three independent αMHC-TRF2 lines were established, expressing TRF2 in a cardiac-specific manner. By immunohistochemistry, the protein product was localized to the nuclei of cardiac myocytes, as expected for the protein and promoter used.

To test for cardiac protection, 10-12 week-old αLMHC-TRF2 mice (n=14) and non-transgenic littermates (n=14) were injected intraperitoneally with 15 mg/kg doxorubicin. By Western blotting 5 days after injection, PARP was decreased in non-transgenic mice treated with doxorubicin, compared to the vehicle-treated, non-transgenic control, and drug-treated TRF2 mice had nearly normal PARP levels (n=5 for each group; P<0.05). By 7 days after doxorubicin injection, 63% of non-transgenic mice had died, with no deaths among the drug-treated αMHC-TRF2 mice. As of day 16, when the experiment was terminated for tissue collection, 36% of the αMHC-TRF2 mice had survived, more than 5-fold greater than the survival after doxorubicin in littermate controls (7%; P<0.01).

### Example 24

### Dominant-Negative TRF2 Triggers Myocyte Apoptosis in vivo and Late-Onset Heart Failure

Disruption of TRF2 function in cultured cells results in DNA damage pathway activation culminating in senescence or apoptosis, depending on context (Oh et al., 2003; Karlseder et al., 1999; and Karlseder et al., 2002). To test this requirement for TRF2 function in the intact adult heart, transgenic mice were created expressing dnTRF2 driven by the same αMHC promoter used for wild-type TRF2. The truncated dnTRF2 protein lacked the N-terminal basic region and the C-terminal DNA-binding Myb motif (TRF2ΔBΔM), and provoked effects in cultured cardiac myocytes and other cells identical to those of antisense interference with TRF2 levels (Oh et al., 2003). Three transgenic lines were established (αMHC-dnTRF2), expressing the protein selectively in cardiac muscle, with appropriate localization to myocyte nuclei.

Dominant-negative TRF2 mice were born in the expected Mendelian ratio, and appeared phenotypically normal through the first six months of life. However, as the αMHC-dnTRF2 mice approached 8-9 months of age, they characteristically developed dilated cardiomyopathy, with four-chamber enlargement, thinning of the ventricular walls, and interstitial fibrosis. The heart-to-body weight ratio of αMHC -dnTRF2 mice (6.9±0.8 mg/g; n=9) was at least 50% greater than in age-matched non-transgenic littermates (4.5±0.1; n=14; P<0.001) or the αMHC-TRF2 mice (4.6±0.2; n=10; P=0.002). Dominant-negative TRF2 increased the prevalence of myocyte apoptosis, compared to the two other groups. Western blotting for PARP further indicated increased apoptosis in dnTRF2 hearts.

Commencing at the age of 36 weeks, mortality was significantly increased by dnTRF2 (N=18), compared with age-matched non-transgenic controls and wild-type TRF2. Furthermore, mortality was increased in all three transgenic lines expressing dnTRF2, with mortality greatest in the highest expressing line. Together with the lack of adverse effects from over-expressing full-length TRF2, this uniformity and dosage-dependence precluded non-specific or insertional effects as the cause of cardiomyopathy and death. Typically, dnTRF2 mice demonstrated potential signs of congestive heart failure including tachypnea and markedly decreased activity.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Abate C, Luk D, Curran T. (1991) Mol Cell Biol 11, 3624-32

Adams, J. W. et al., Proc. Natl. Acad. Sci. U.S.A. 95, 10140-10145 (1998).

Akli, S., etal., Circ. Res. 85, 319-328 (1999).

Allshire et al., (1988) Nature, 332:656-659.

Allsopp, R. C. et al. Nat Med 9, 369-71 (2003).

Araki, K., et al., Proc. Natl. Acad. Sci. U.S.A. 92, 160-164 (1995).

Arcone, et al., (1988) Nucl. Acids Res., 16(8), 3195-3207.

Aza-Blanc, P. et al., Mol Cell 12, 627-37 (2003).

Bacchetti, S. (1992) EMBO J. 11, 1921-1929.

Baichwal and Sugden, (1986) In, Gene Transfer, pp. 117-148.

Bakkenist, C. J. et al., Nature 421, 499-506 (2003).

Bartunek, J., et al., (2000) Circulation 101, 2854-62.

Becker, E. et al., Mol. Cell. Biol. 20, 1537-45 (2000).

Benvenisty and Neshif, (1986) Proc. Nat'l Acad. Sci. USA, 83,9551-9555.

Berzal-Herranz et al, (1992) Genes Dev.; 6,129-34.

Blackburn et al., (1978) J. Mol. Biol., 120:33-53.

Blackburn et al., (1984) Cell, 36:447-458.

Blackburn, E. H. (2001) Cell 106, 661-73.

Blasco, M. A., et al., (1995) Science 269, 1267-1270.

Bodner et al., (1998) Science, 279:349-352.

Bohmann et al., (1989) Cell. Nov 17; 59(4),709-17.

Brown, Nature, (1986) 338:774-776.

Carter and Flotte, (1995) Ann. N.Y. Acad. Sci., 770; 79-90.

Cech et al., (1981) Cell.; 27(3 Pt 2),487-96.

Chang, L. et al., Nature 410, 37-40. (2001).

Chang, S. & DePinho, R. A. (2002) Proc Natl Acad Sci USA 23, 23.

Chatteijee, et al., (1995) Ann. N.Y. Acad. Sci., 770,79-90.

Chowrira et al, (1994) J Biol Chem.; 269,25856-64.

Chowrira et al., (1993) J Biol Chem. Sep 15; 268(26),19458-62.

Coffin, (1990) In: Virology, ed., New York: Raven Press, pp. 1437-1500.

Counter, C. M., et al., (1988) Gene, 68,1-10.

Courey et al., (1988) Cell.; 55(5),887-98.

Cross et al., (1989) Nature, 338:771-774.

Dan, I. et al., FEBS Lett 469, 19-23 (2000).

Dan, I., et al., Trends Cell Biol 11, 220-30 (2001).

D'Angelo, D. D. et al., Proc. Natl. Acad. Sci. U.S.A. 94, 8121-8126 (1997).

de Lange, T. (2002) Oncogene 21, 532-540.

DePinho, R. A. & Wong, K. K. J Clin Invest 111, S9-14 (2003).

Ding, B., et al., (1984) Proc. Nat'l Acad. Sci. USA, 81,7529-7533.

Dowd, N. P., et al., J Clin Invest 108, 585-90 (2001).

Dwarki et al., Proc. Natl. Acad. Sci. USA, 92:1023-1027 (1995).

Elledge, S. J. (1996) Science. 274, 1664-72.

Enoch, T. & Norbury, C. (1995) Trends Bioch Sci. 20, 426-430.

Fechheimer et al., (1987) Proc. Nat'l Acad. Sci. USA, 84,8463-8467.

Felgner and Ringold, Science, 337:387-388 (1989).

Felgner et. al., Proc. Natl. Acad. Sci. US.A., 84:7413-7417 (1987).

Ferrari et al., (1996) J. Virol., 70,3227-3234.

Fisher et al., (1996) J. Virol., 70,520-532.

Flotte et al., Proc. Nat'l Acad. Sci. USA, 90,10613-10617, (1993).

Forster and Symons (1987) Cell.; 50,9-16.

Fraley et al., (1979) Proc Nat'l Acad. Sci. USA, 76,3348-3352.

Frey, N. & Olson, E. N. (2003) Annu. Rev. Physiol. 65, 45-79.

Friedman et al., (1990) Genes Dev.; 4,1416-26.

Fu, C. A. et al., J Biol Chem 274, 30729-37 (1999).

Gaussin, V. et al., Proc. Natl. Acad. Sci. U.S.A. 99, 2878-2883 (2002).

Gerster et al., (1990) EMBO J.; 9,1635-43.

Ghosh and Bachhawat, (1991) In: Liver Diseases, Targeted Diagnosis and Therapy Using Specific Receptors and Ligands. pp. 87-104.

Giet et al, (2001) J Cell Biol.; 152,669-82.

Goodman et al. (1994), Blood, 84,1492-1500.

Gopal et al (1985), Mol. Cell Biol., 5,1188-1190.

Gossen and Bujard, (1992) Proc. Nat'l Acad. Sci. USA, 89,5547-5551.

Gossen et al., (1995) Science, 268,1766-1769.

Graham and van der Eb, (1973) Virology, 52,456-467.

Greider and Blackburn, (1985) Cell, 43:405-413.

Grepin, C., et al., (1997) Development 124, 2387-2395.

Griffith, J. D., et al., (1999) Cell 97, 503-514.

Hahn, W. C., et al., (1999) Nature Med. 5, 1164-1170.

Hammond et al., (2001) Nat Rev Genet. 2,110-9.

Harland and Weintraub, (1985) J. Cell Biol., 101,1094-1099,

Haseloff and Gerlach (1988) Nature.; 334,585-91.

Hay et al., (1984) J. Mol. Biol., 175,493-510.

Hayakawa, Y. et al., Circulation 108, 3036-41 (2003).

He, T. C., et al., (1998) Proc. Natl. Acad. Sci. U.S.A. 95,2509-2514.

He, T. C. et al., Proc. Natl. Acad. Sci. U.S.A. 95, 2509-2514 (1998).

Hearing and Shenk, (1983) J. Mol. Biol. 167,809-822,

Hearing et al., J. (1987) Virol., 67,2555-2558.

Hemann, M. T., et al., (2001) Mol. Biol. Cell 12, 2023-30.

Hirota, H., et al., (1999) Cell 97, 189-198.

Hope et al., (1987) EMBO J. 6(9),2781-4.

Jackson, K. A., et al., (2001) J. Clin. Invest. 107, 1395-1402.

Johnson, G. L. et al., Science 298, 1911-2 (2002).

Joyce et al., (1989) Nature, 338,217-244,

Kageyama et al., (1987) J. Biol. Chem., 262,2345-2351.

Kajstura, J., et al., (2003) EMBO J. 22, 131-139.

Kanai-Azuma, M. et al., Mech Dev 89, 155-9 (1999).

Kaneda et al., (1989) Science, 243,375-378.

Kang, P. M. & Izumo, S. (2000) Circ. Res. 86, 1107-1113.

Kaplitt et al., (1994) Nat'l Genet., 8,148-153.

Kaplitt et al., Molec. Cell. Neurosci., 2:320-330 (1991)

Karlseder, J., et al., (1999) Science 283, 1321-1325.

Karlseder, J., et al., Science 283, 1321-1325. (1999).

Karlseder, J., et al., Science 295, 2446-9. (2002).

Kasis et al., Proc. Natl. Acad. Sci. U.S.A., 87:473 (1990).

Kato et al, (1991) J. Biol. Chem., 266,3361-3364.

Keefe, D. L. Semin Oncol 28, 2-7 (2001).

Kelleher, C., et al., (2002) Trends Biochem. Sci. 27, 572-579.

Kessler et al., (1996) Proc. Nat'l Acad. Sci. USA, 93,14082-14087.

Kiaris, H. & Schally, A. V. (1999) Proc. Natl. Acad. Sci. U.S.A. 96, 226-31.

Kim and Cech (1987) Proc Natl Acad Sci USA. 84(24),8788-92.

Kishimoto, K., et al., J. Biol. Chem. 275, 7359-64. (2000).

Klein et al., (1987) Nature, 327,70-73.

Klobutcher etal., (1981) Proc. Natl. Acad. Sci. USA, 78:3015-3019.

Koeberl et al., (1997) Proc. Nat'l Acad. Sci. USA, 94,1426-1431.

Koh, G. Y., et al., (1995) J. Clin. Invest. 96, 2034-2042.

Kubasiak, L. A., et al., (2002) Proc Natl Acad Sci USA 99, 12825-30.

Lee, H. W., et al., (1998) Nature 392, 569-74.

Lei, K. et al., Proc Natl Acad Sci USA 100, 2432-7 (2003).

Leri, A., et al., (1991) Gene 101, 195-202.

Levade, T. et al., Circ. Res. 89, 957-968 (2001).

Liang, Q. et al., EMBO J. 22, 5079-5089 (2003).

Liao, W. C. et al., J Biol Chem 274, 17908-17 (1999).

Lieber et al., (1995) Mol Cell Biol. 15,540-51.

Lorenz, J. N. & Dom, G. W. (2002) Nat. Med. 10, 10.

Lundblad et al., (1989) Cell, 83:633-643.

Ma et al., (1987) 50,137-42.

Mackey et al., Proc. Natl. Acad. Sci. U.S.A., 85:8027-8031 (1988).

MacLellan, W. R. & Schneider, M. D. (2000) Annu. Rev. Physiol. 62,289-320.

Mann et al., (1983) Cell, 33,153-159.

Manning, G., et al., Science 298, 1912-34 (2002).

Martin et al., (1990) Genes Dev.; 4,1886-98.

McCown et al., (1996) Brain Res., 713,99-107.

McEachem, M. J., et al., (2000) Annu. Rev. Genet. 34, 331-358.

McGowan, C. H. (2002) BioEssays 24, 502-511.

Melchionna, R., et al., (2000) Nat Cell Biol 2, 762-5.

Mermod et al., (1989) Cell. 58,741-53.

Michael, L. H. et al., Am. J. Physiol. 269, H2147-H2154 (1995).

Michel and Westhof, (1990) J. Mol. Biol., 216,585-610.

Minamino, T. et al., Proc. Natl. Acad. Sci. U.S.A. 99, 3866-3871 (2002).

Mizukami et al., (1996) Virology, 217,124-130.

Moyzis et al., (1988) Proc. Natl. Acad. Sci. USA, 85:6622-6626.

Muller-Immergluck et al., (1990) EMBO J.; 9,1625-34.

Multani, A. S., et al., (2000) Neoplasia 2, 339-45.

Nakano, K., et al., Exp Cell Res 287, 219-27 (2003).

Nicolas and Rubenstein, (1988) In: Vectors: A survey of molecular cloning vectors and their uses, pp. 493-513,

Nicolau and Sene, (1982) Biochim. Biophys. Acta, 721,185-190.

Nicolau et al., (1987) Methods Enzymol., 149,157-176.

Oh, H. et al., Proc Natl Acad Sci USA 100, 12313-8 (2003).

Oh, H. et al., Proc Natl Acad Sci USA 100, 5378-5383 (2003).

Oh, H., Taffet, et al., (2001) Proc. Natl. Acad. Sci. U.S.A. 98, 10308-10313.

Oka et al, Gene (1980) 10:301.

Oliviero et al., (1987) EMBO J., 6,1905-1912.

Ono, K. et al., J Biol Chem 25, 25 (2001).

Opresko, P. L., et al., (2002) J. Biol. Chem. 277, 41110-41119.

Panta, G. R. et al., Mol Cell Biol 24, 1823-1835 (2004).

Paskind et al., (1975) Virology, 67,242-248.

Pasumarthi, K. B. & Field, L. J. (2002) Circ. Res. 90, 1044-1054.

Perrotta and Been (1992) Biochemistry. 31,16-21.

Petrich, B. G., et al., Faseb J 17, 749-51 (2003).

Ping et al., (1996) Microcirculation, 3,225-228.

Pluta et al., (1989) Nature, 337:429-433.

Poli and Cortese, (1989) Proc. Nat'l Acad. Sci. USA, 86,8202-8206.

Potter et al., (1984) Proc. Nat'l Acad. Sci. USA, 81,7161-7165.

Prowse and Baumann, (1988) Mol Cell Biol, 8,42-51.

Prowse, K. R. & Greider, C. W. (1995) Proc. Natl. Acad. Sci. U.S.A. 92, 4818-4822.

Radler et al., (1997) Science, 275,810-814.

Ramirez, R., et al., (2003) J. Biol. Chem. 278, 836-42.

Reed, J. C. & Patemostro, G. (1999) Proc. Natl. Acad. Sci. U.S.A. 96, 7614-7616.

Reinhold-Hurek and Shub, (1992) Nature, 357,173-176.

Renan, M. J. (1990) Radiother Oncol., 19, 197-218.

Richards et al., (1988) Cell, 53:127-136.

Ridgeway, (1988) In: Vectors: A survey of molecular cloning vectors and their uses, pp. 467-492.

Ron, et al., (1991) Mol. Cell. Biol., 2887-2895.

Roux et al., (1989) Proc. Nat'l Acad. Sci. USA, 86,9079-9083.

Rudolph, K. L. et al. Cell 96, 701-712 (1999).

Sadoshima, J., et al., (2002) J. Clin. Invest. 110, 271-9.

Sadowski et al., (1988) Nature. 335,563-4.

Sakata, Y., Circulation 97, 1488-1495 (1998).

Samulski et al., J. Virol., 61:3096-3101 (1987)

Samulski et al., J. Virol., 63:3822-3828 (1989)

Sano, M. et al., Nat. Med. 8, 1310-1317 (2002).

Sarver et al., (1990) Science, 247,1222-1225.

Scanlon et. al., (1991) Proc. Nat'l Acad. Sci. USA, 88,10591-10595.

Shibuya, H. et al., EMBO J. 17, 1019-1028 (1998).

Shibuya, H. et al., Science 272, 1179-1182 (1996).

Shiloh, Y., Nat Rev Cancer 3, 155-68 (2003).

Sioud et al., (1992) J Mol Biol.; 223,831-5.

Sivasubramanian, N. et al., Circulation 104, 826-31. (2001).

Stewart, S. A. & Weinberg, R. A. (2002) Oncogene 21, 627-630.

Stratford-Perricaudet et al., J. Clin. Invest., 90:626-630 (1992).

Su, Y. C., et al., EMBO J. 16, 1279-90 (1997).

Subramaniam, A. et al., J. Biol. Chem. 266, 24613-24620 (1991).

Suematsu, N. et al., Circulation 107, 1418-23 (2003).

Szostak et al., (1982) Cell, 36:459-568.

Takai, H., et al., Curr Biol 13, 1549-56 (2003).

Temin et al., (1986) In: Gene Transfer, Kucherlapati (ed.), New York: Plenum Press, pp. 149-188.

Theill et al., (1989) Nature; 342,945-8.

Thompson, Thromb. and Haemostatis, 66:119-122 (1991).

Tibbetts et. al. (1977) Cell, 12,243-249.

Tur-Kaspa et al., (1986) Mol. Cell Biol., 6,716-718.

Van der Ploeg et al., (1984) Cell 36:459-468.

Vlahos, C. J., et al., Nat Rev Drug Discov 2, 99-113 (2003).

Watt et al., (1986) Proc. Nat'l Acad. Sci., 83, 3166-3170.

Weinert, T. & Lundblad, V. (1999) Nat. Genet. 21, 151-152.

Wencker, D. et al., Clin Invest 111, 1497-504 (2003).

Wilson et al., (1990) Mol. Cell. Biol., 6181-6191.

Wong et al., (1980) Gene, 10,87-94.

Wong, K. K., et al., (2000) Nat Genet 26, 85-8.

Wright, J. H. et al., Mol Cell Biol 23, 2068-82 (2003).

Wu and Wu, (1987) J. Biol. Chem., 262,4429-4432.

Wu and Wu, (1988) Biochem., 27,887-892.

Wu, M. N. et al. (1999) Neuron; 23,593-605.

Wu, M. N. et al. (1997) EMBO J; 17,127-39.

Xiao et al., (1996) J. Virol., 70,8098-8108.

Yamamoto, S. et al., Clin Invest 111, 1463-74 (2003).

Yang et al., (1990) Proc. Nat'l Acad. Sci. USA, 87,9568-9572.

Yao, Z. et al., J. Biol. Chem. 274, 2118-2125 (1999).

Yao, Z. B. et al., J Biol Chem 272, 32378-32383 (1997).

Yuan and Altman (1994) Science; 263,1269-73.

Yuan et al., (1992) Proc Natl Acad Sci USA; 89,8006-10.

Yussman, M. G., et al., (1988) Mol. Cell. Biol., 2394-2401.

Yussman, M. G. et al., Nat. Med. 8, 725-730 (2002).

Zakian, Science (1995) 270:1601.

Zhang, D. et al., Nat. Med. 6, 556-563 (2000).

Zhong et al., (1992) Mol. Cell. Biol., 12:4834-4943.

Zhu, W. et al., Circulation 100, 2100-7 (1999).

Zhu, X. D., et al., (2000) Nat. Genet. 25, 347-352.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of enhancing the survival of a cell comprising the steps of administering to the cell a composition that regulates telomere stability in the cell.

2. The method of claim 1, wherein the cell is in a tissue.

3. The method of claim 3, wherein the tissue is in a human.

4. The method of claim 1, wherein the cell is a cardiomyocyte.

5. The method of claim 1, wherein the cell is under oxidative stress.

6. The method of claim 1, wherein the composition comprises a modulator of telomeric repeat binding factor-2 (TRF2).

7. The method of claim 6, wherein the modulator is telomerase reverse transcriptase (TERT).

8. The method of claim 6, wherein the modulator is an inhibitor of hematopoietic progenitor kinase/gerrninal center kinase like kinase (HGK).

9. The method of claim 1, wherein the composition comprises a modulator of cell cycle checkpoint kinase 2 (Chk2).

10. A method of treating a subject suffering from a cardiovascular disease comprising the step of administering to the subject an effective amount of a composition to regulate telomere stability, wherein the effective amount increases cardiomyocyte survival.

11. The method of claim 10, wherein the composition comprises a modulator of TRF2.

12. The method of claim 11, wherein the modulator is TERT.

13. The method of claim 11, wherein the modulator is an inhibitor of HGK.

14. The method of claim 10, wherein the composition comprises a modulator of Chk2.

15. The method of claim 10, wherein said cardiovascular disease is selected from the group consisting of coronary artery disease, myocardial infarction, heart failure, ischemic heart disease, and angina.

16. The method of claim 15, wherein said cardiovascular disease is myocardial infarction.

17. The method of claim 16, wherein said myocardial infarction is caused by arterial obstruction.

18. The method of claim 10, wherein said cardiovascular disease is caused by oxidative stress on cardiomyocytes.

19. The method of claim 10, wherein said cardiovascular disease is caused by telomere loss and/or telomere dysfunction in cardiomyocytes.

20. The method of claim 19, wherein said telomere loss and/or dysfunction results in apoptosis.

21. The method of claim 20, wherein said apoptosis is associated with check point kinase Chk2 activation.

22. The method of claim 11, wherein said modulator increases activity of said TRF2.

23. The method of claim 11 wherein said modulator increases the expression of said TRF2.

24. The method of claim 11, wherein said modulator increases the stability of said TRF2.

25. The method of claim 10, wherein said composition comprises an expression vector having a polynucleotide sequence encoding a TRF2 protein.

26. The method of claim 14, wherein said modulator inhibits Chk2 activity.

27. The method of claim 14, wherein said modulator reduces expression of Chk2.

28. The method of claim 14, wherein said modulator increases degradation of Chk2.

29. The method of claim 14, wherein said modulator destabilizes Chk2.

30. A method of treating a subject suffering from a myocardial infarction comprising the step of administering to the subject an effective amount of a composition to regulate telomere stability, wherein the effective amount increases cardiomyocyte survival.
